(19) United States
(12) Patent Application Publication
(10) Pub. No.: US 2022/0160905 A1
(43) Pub. Date: May 26, 2022

(54) FLUORINATION METHOD

(71) Applicant: OXFORD UNIVERSITY INNOVATION LIMITED, Oxfordshire (GB)

(72) Inventors: Veronique Gouverneur, Oxfordshire (GB); Choon Wee Kee, Oxfordshire (GB); Osman Tack, Oxfordshire (GB); Thomas Charles Wilson, Oxfordshire (GB)

(73) Assignee: OXFORD UNIVERSITY INNOVATION LIMITED, Oxfordshire (GB)

(21) Appl. No.: 17/275,915

(22) PCT Filed: Sep. 13, 2019
(86) PCT No.: PCT/GB2019/052577

§ 371 Date: Mar. 12, 2021

(30) Foreign Application Priority Data
Sep. 14, 2018 (GB) 1815013.6

(51) Int. Cl. A61K 51/08; C07K 1/107; C07D 209/20; C07B 59/00

(52) U.S. Cl. CPC A61K 51/08; C07K 1/1077; C07B 2200/05; C07B 59/008; C07D 209/20

(57) ABSTRACT

The invention relates to a process for producing a compound comprising the anion [CF₂¹⁸FSO₂]−, which process comprises treating a difluorocarbene source with (i) a source of ¹⁸F⁻ and (ii) a source of SO₂. The invention relates to a compound which comprises that anion. The invention also relates to the use of the compound comprising the anion [CF₂¹⁸FSO₂]⁻ to produce a compound comprising an ¹⁸F-trifluormethyl functionalised aromatic group. Compounds comprising an ¹⁸F-trifluoromethyl functionalised aromatic group are also the subject of the present invention.

### FIELD OF THE INVENTION

The invention relates to a process for producing a compound comprising the anion [CF₂¹⁸FSO₂]⁻, and to the compound itself, which comprises that anion. The invention also relates to the use of the compound comprising the anion [CF₂¹⁸FSO₂]⁻ to produce a compound comprising an ¹⁸F-trifluoromethyl functionalised aromatic group. Compounds comprising an ¹⁸F-trifluoromethyl functionalised aromatic group are also the subject of the present invention.

### BACKGROUND TO THE INVENTION

Positron Emission Tomography (PET) is a powerful molecular imaging technique for diagnosis, monitoring disease progression, studying biological processes in vivo and investigating the efficacy of drugs. Among all the radioisotopes required for the preparation of PET probes, ¹⁸F is the most widely used and clinically relevant radionuclide. Due to its short half-life (t_{1/2} 109.7 min), ¹⁸F must be incorporated into tracer molecules at a late stage of the overall synthesis process. Additional challenges imposed by radiochemistry include low reaction concentrations, solvent compatibility, and cyclotron-produced ¹⁸F sources being limited to ¹⁸F-fluoride and [¹⁸F]F₂ only. These constraints are particularly stringent for biomolecules.

Peptides and proteins display excellent binding specificities to targets linked to disease states. Much effort has been made towards developing ¹⁸F-labeling methods for these probes (Richter, S., et al., Molecules 2014, 19 (12), 20536-20556). ¹⁸F has been incorporated into pre-functionalized peptides and proteins via C-¹⁸F, B-¹⁸F and Si-¹⁸F bond formation, or chelation with Al-¹⁸F (see Bernard-Gauthier, V. et al., Biomed Res. Int. 2014, 2014, 454503; Laverman, P. et al. J. Label. Compd. Radiopharm. 2014, 57 (4), 219-223; Cornilleau, T. et al., Org. Lett. 2015, 17 (2), 354-357; Perrin, D. M. Acc. Chem. Res. 2016, 49 (7), 1333-1343). Alternatively, an ¹⁸F-labeled prosthetic group is prepared prior to bioconjugation under mild reaction conditions (see Marik, J., et al. Tetrahedron Lett. 2006, 47 (37), 6681-6684; Gao, Z. et al. J. Am. Chem. Soc. 2013, 135 (37), 13612-13615; Jacobson, O. et al., X. Bioconjug. Chem. 2015, 26 (10), 2016-2020; Way, J. D. et al., Chem. Commun. 2015, 51 (18), 3838-3841; Chiotellis, A. et al., T. L. Chem. Commun. 2016, 52 (36), 6083-6086). Attaching the ¹⁸F-labeled prosthetic group may be achieved by synthetic manipulation of the biomolecule to enable attachment of the ¹⁸F-prosthetic group. Alternatively, the ¹⁸F-labeled prosthetic group may be attached to the peptide or protein by taking advantage of the inherent nucleophilicity of amino acid side-chains such as cysteine thiols (see Chalker, J. M. et al., Chem.—An Asian J. 2009, 4 (5), 630-640) or lysine amines. Existing ¹⁸F-labeling strategies for unmodified peptides or proteins require the radiosynthesis of ¹⁸F-prosthetics followed by a bioconjugation process that harnesses the reactivity of heteroatom lone-pair nucleophiles such as cysteine thiols or lysine amines. This limits the nature of functional groups on which ¹⁸F-functionalisation can be performed.

Although tolerated for many applications, these ¹⁸F-labeling strategies can significantly alter the structure of native probes. Efficacy and/or function can be adversely affected, for example by changing pharmacokinetic profiles, disrupting hydrogen-bonding interactions or disturbing local polarity. As such, the discovery of ¹⁸F-labeling methods targeting native aromatic amino acid residue in peptides or proteins with either ¹⁸F, the smallest possible ¹⁸F-tag, or a minimally sized ¹⁸F-prosthetic (e.g. [¹⁸F]CF₃) would be of considerable value to many scientists.

A method for modifying cysteine thiols with 5-¹⁸F-(trifluoromethyl)dibenzothiophenium trifluoromethanesulfonate to allow ¹⁸F-trifluoromethylation of native peptides has been reported by Verhoog et al. (Verhoog et al., ¹⁸F-Trifluoromethylation of unmodified peptides with 5-18F-(Trifluoromethyl)dibenzothiophenium Trifluoromethanesulfonate, J. Am. Chem. Soc. 2018, 140, 1572-1575). This method only however permits functionalization of nucleophilic thiol groups, thereby limiting the possible sites of functionalization and the types of molecules that can be functionalised.

In Imiolek et al. (Imiolek, M., Selective Radical Trifluoromethylation of Native Residues in Proteins, J. Am. Chem. Soc 2018, 140, 1568-1571) tuned radical chemistry is applied to program C—H ¹⁹F-trifluoromethylation of innately electron rich residues in proteins. Sodium trifluoromethanesulfinate (NaTFMS, Langlois' reagent) displayed selective reactivity for tryptophan under redox initiation. However, as outlined above, a stringent set of considerations apply when preparing and using “hot” ¹⁸F reagents as opposed to their ¹⁹F counterparts. In particular, methods must be developed to synthesise the reagents quickly and in high yield from cyclotron ¹⁸F sources, i.e. ¹⁸F⁻. The reagents synthesised must then be capable of reacting quickly and cleanly with the molecule to be labelled. Due to the short half life of ¹⁸F, speed and simplicity of reaction is critical to ensure that ¹⁸F can be effectively used to monitor biological processes/systems, for instance through PET.

Ichiishi et al. (Ichiishi, N., Protecting group free radical C—H trifluoromethylation of peptides; Chem. Sci., 2018, 9 (17), 4168-4175) demonstrated that Zn(TFMS)₂ (Baran's reagent) when activated with a stoichiometric oxidant or via visible photoredox catalysis, enables trifluoromethylation of tyrosine in peptides that do not contain tryptophan residues. This paper is concerned with standard ¹⁹F chemistry only therefore does not address the particular set of problems for developing a “hot” ¹⁸F version of the TFMS ion as outlined above.

Routes towards trifluoromethanesulfinic acid salts are known, including metal or electro-reduction of a mixture of SO₂ and CF₃Br in DMF (see Folest, J.-C et al., Synth. Commun. 1988, 18 (13), 1491-1494), the treatment of CF₃Cl with sodium dithionite (Na₂S₂O₄) (see Cao, H. P. et al., J. Fluor. Chem. 2007, 128 (10), 1187-1190), or multistep synthesis featuring a key β-elimination process from trifluoromethylsulfone precursors (see Langlois, B. R. et al., C. J. Fluor. Chem. 2007, 128 (7), 851-856). A schematic of each of these known routes is provided below in scheme 1A.

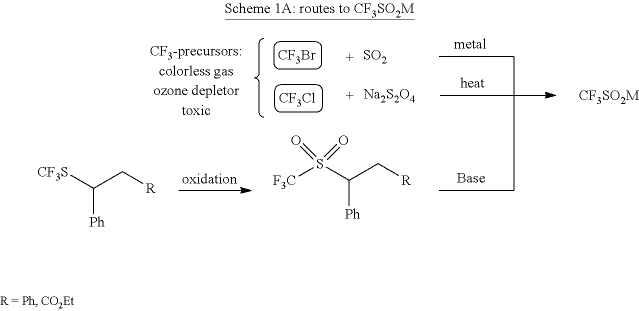

For radiochemistry, these approaches are unsuitable because they are convoluted. First, they would require a radiosynthetic route towards the necessary [¹⁸F]CF₃-precursor (for instance CF₂¹⁸FBr or CF₂¹⁸FCl), and then one or more reactions post-labeling would be needed to form the [CF₂¹⁸FSO₂][M] product. It is not thought that these routes could successfully be employed to produce [CF₂¹⁸FSO₂]⁻ in the first place, but even if any one of them could successfully be used the route would still be too convoluted and time-consuming for radiochemistry. In addition, further steps would be required to functionalise the molecule of interest, giving a minimum of three steps in the overall reaction pathway to the ¹⁸F-labelled product. Such a route would be too time consuming and complex given the short half-life of ¹⁸F; by the time the desired ¹⁸F-labelled product were synthesized positron emission would have decayed to levels unsuitable for use in PET imaging.

### SUMMARY OF THE INVENTION

The present invention provides access for the first time, to compounds comprising the anion [CF₂¹⁸FSO₂]⁻. This has been achieved by developing a new process that is successfully able to produce ¹⁸F-trifluoromethanesulfinate, in a fast and reliable synthesis. The synthesis is performed by combining multiple reactants: a source of ¹⁸F-fluoride, a difluorocarbene source, and a source of SO₂. The invention therefore addresses the issues discussed above, by providing a quick and facile route to ¹⁸F-trifluoromethanesulfinate, which may be performed in a single step. The simple one-pot nature of the process of the invention makes it highly suitable for preparing this “hot” reagent quickly, so that it can then be used to radiolabel molecules of interest, such as peptides and proteins.

The invention therefore provides a fast and reliable route for synthesis of [CF₂¹⁸FSO₂]⁻, to permit facile radiolabelling of aromatic groups. Indeed, the “hot” ¹⁸F-trifluoromethanesulfinate reagent permits direct [¹⁸F]CF₃-incorporation at aromatic groups, such as those that are present in tryptophan and tyrosine residues in unmodified peptides as complex as human insulin. This functionalization process utilises electrophilic radical chemistry to target (hetero)aromatic residues with an ¹⁸F-trifluoromethyl group. The ability of ¹⁸F-trifluoromethanesulfinate to enable selective C—H ¹⁸F-trifluoromethylation of aromatic groups, such as those found on amino acid residues within unmodified peptides and proteins is also demonstrated herein.

Accordingly, the invention provides a process for producing a compound comprising the anion [CF₂¹⁸FSO₂]⁻, which process comprises treating a difluorocarbene source with (i) a source of ¹⁸F⁻ and (ii) a source of SO₂.

The invention also provides a compound comprising the anion [CF₂¹⁸FSO₂]⁻.

The invention also provides a process for producing a compound comprising an ¹⁸F-trifluoromethyl functionalised aromatic group, which process comprises contacting a compound comprising an aromatic group with a compound comprising the anion [CF₂¹⁸FSO₂]⁻ in the presence of an activator for trifluoromethyl radical formation.

The present invention also provides a compound comprising an ¹⁸F-trifluoromethyl functionalised aromatic group.

The invention also provides a compound comprising an ¹⁸F-trifluoromethyl functionalised aromatic group for use in a method for treatment of the human or animal body by therapy or for use in a diagnostic method practised on the human or animal body.

The invention also provides a method of imaging a subject, comprising administering to the subject a compound comprising an ¹⁸F-trifluoromethyl functionalised aromatic group, or a pharmaceutically acceptable salt thereof, and imaging the subject by positron emission tomography (PET).

### BRIEF DESCRIPTION OF THE FIGURES

FIGS. 1, 2, 3 and 4 show characterising mass spectrometry data for CF₃-functionalised insulin. FIG. 1 shows mass spectrometry data for Insulin chain A Y14-CF₃. FIG. 2 shows mass spectrometry data for Insulin chain A Y19-CF₃. FIG. 3 shows mass spectrometry data for Insulin chain B Y16-CF₃. FIG. 4 shows mass spectrometry data for Insulin chain B Y26-CF₃.

FIG. 5 shows an overlay of a radiotrace for [¹⁸F]NH₄SO₂CF₃ and the UV trace of the authentic reference sample (220 nm).

FIG. 6 shows the calibration curve for [¹⁹F]NH₄SO₂CF₃ for molar activity determination FIGS. 7 and 8 are overlays of the crude radio-traces for the products of the reaction of L-tyrosine with [¹⁸F]NH₄SO₂CF₃ and the UV (220 nm) trace of the authentic reference sample.

FIGS. 9 and 10 are overlays of the crude radio-traces for the products of the reaction of L-tryptophan with [¹⁸F]NH₄SO₂CF₃ and UV (220 nm) traces of the authentic reference sample.

FIG. 11 is an overlay of the UV (220 nm) and radio trace of the crude reaction for Table 28 Entry 2.

FIGS. 12 and 13 are overlays of the crude radio-traces for the products of the reaction of H-Tyr-Trp-OH with [¹⁸F]NH₄SO₂CF₃ and the UV (220 nm) trace of the authentic reference sample.

FIGS. 14, 15 and 16 show overlays of the crude radio-traces for the products of the reaction of H-Trp-Tyr-OH with [¹⁸F]NH₄SO₂CF₃ and the UV (220 nm) trace of the authentic reference sample.

FIGS. 17, 18 and 19 show overlays of the crude radio-traces for the products of the reaction of H-Phe-Tyr-OH with [¹⁸F]NH₄SO₂CF₃ and the UV (220 nm) trace of the authentic reference sample. Percentage is estimated from 19F NMR.

FIGS. 20, 21 and 22 show overlays of the crude radio-traces for the products of the reaction of H-Phe-Trp-OH with [¹⁸F]NH₄SO₂CF₃ and the UV (220 nm) trace of the authentic reference sample.

FIGS. 23 and 24 are overlays of the crude radio-traces for the products of the reaction of H-Met-Trp-OH with [¹′F]NH₄SO₂CF₃ and the UV (220 nm) trace of the authentic reference sample.

FIGS. 25, 26 and 27 show overlays of the crude radio-traces for the products of the reaction of H-Met-Tyr-OH with [¹⁸F]NH₄SO₂CF₃ and the UV (220 nm) trace of the authentic reference sample.

FIGS. 28 and 29 provide overlays of the crude radio-traces for the products of the reaction of H-Tyr-His-OH with [¹⁸F]NH₄SO₂CF₃ and the UV (220 nm) trace of the authentic reference sample.

FIGS. 30, 31 and 32 provide overlays of the crude radio-traces for the products of the reaction of H-His-Trp-OH with [¹⁸F]NH₄SO₂CF₃ and the UV (220 nm) trace of the authentic reference sample.

FIGS. 33 and 34 provide overlays of the crude radio-traces for the products of the reaction of H-Glu-Trp-OH with [¹⁸F]NH₄SO₂CF₃ and UV (220 nm) trace of the authentic reference sample.

FIG. 35 shows an overlay of radio-trace of isolated product (2-CF₃) for H-Glu-Trp-OH reaction with [¹⁸F]NHaSO₂CF₃ and UV (220 nm) trace of authentic reference.

FIGS. 36 and 37 provide overlays of the crude radio-traces for the products of the reaction of Angiotensin (1-7) with [¹⁸F]NH₄SO₂CF₃ and the UV (220 nm) trace of the authentic reference sample.

FIG. 38 provides an overlay of the crude radio-trace for the products of the reaction of Melittin with [¹⁸F]NH₄SO₂CF₃ and the UV (220 nm) trace of the authentic reference sample.

FIG. 39 provides an overlay of the crude radio-trace for the product of the reaction of Somatostatin-14 with [¹⁸F]NH₄SO₂CF₃ and the UV (220 nm) trace of the authentic reference sample.

FIG. 40 is an overlay of radio-trace of isolated product (2-CF₃) of the reaction of Somatostatin-14 with [¹⁸F]NH₄SO₂CF₃ and the UV (220 nm) trace of the authentic reference.

FIGS. 41 and 42 provide overlays of the crude radio-traces for the products of the reaction of Endomorphin 1 with [¹⁸F]NH₄SO₂CF₃ and the UV (220 nm) trace of the authentic reference sample.

FIG. 43 is an overlay of the radio-trace of isolated product (2-CF₃) for the reaction of Endomorphin 1 with [¹⁸F]NH₄SO₂CF₃ and the UV (220 nm) trace of authentic reference.

FIG. 44 provides an overlay of the crude radio-trace for Insulin Chain A Y19-CF₃ of the product of the reaction of Insulin with [¹⁸F]NH₄SO₂CF₃ and the UV (220 nm) trace of the authentic reference sample.

FIG. 45 provides an overlay of the crude radio-trace for Insulin Chain B Y16-CF₃ and Chain B Y26-CF₃ of the product of the reaction of Insulin with [¹⁸F]NH₄SO₂CF₃ and the UV (220 nm) trace of the authentic reference sample.

FIG. 46 provides an overlay of the crude radio-trace for Insulin Chain A Y14-CF₃ of the product of the reaction of Insulin with [¹⁸F]NH₄SO₂CF₃ and the UV (220 nm) trace of the authentic reference sample.

FIG. 47 provides an overlay of the crude radio-trace for the product of the reaction of cyclo(-Arg-Gly-Asp-D-Tyr-Lys) with [¹⁸F]NH₄SO₂CF₃ and the UV (220 nm) trace of the reference sample (¹⁹F-trifluoromethyl functionalised cyclo(-Arg-Gly-Asp-D-Tyr-Lys)).

FIG. 48 provides the ¹H NMR spectrum for ¹⁹F-trifluoromethyl functionalised cyclo(-Arg-Gly-Asp-D-Tyr-Lys).

FIG. 49 provides the ¹⁹F NMR spectrum for ¹⁹F-trifluoromethyl functionalised cyclo(-Arg-Gly-Asp-D-Tyr-Lys).

FIG. 50 provides the time-of-flight mass spectrometry results for ¹⁹F-trifluoromethyl functionalised cyclo(-Arg-Gly-Asp-D-Tyr-Lys).

### DETAILED DESCRIPTION OF THE INVENTION

### Definitions

The term “alkyl group”, as used herein, refers to a substituted or unsubstituted, straight or branched chain saturated hydrocarbon radical. Typically an alkyl group is C₁₋₂₀ alkyl, or C₁₋₁₀ alkyl, for example methyl, ethyl, propyl, butyl, pentyl, hexyl, heptyl, octyl, nonyl or decyl (including straight or branched chain isomers thereof), or C₁₋₆ alkyl, for example methyl, ethyl, propyl, butyl, pentyl or hexyl (including straight or branched chain isomers thereof), or C₁₋₄ alkyl, for example methyl, ethyl, i-propyl, n-propyl, t-butyl, s-butyl or n-butyl. When an alkyl group is substituted it typically bears one or more substituents selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Examples of substituted alkyl groups include haloalkyl, hydroxyalkyl, aminoalkyl, alkoxyalkyl and alkaryl groups. The term alkaryl, as used herein, pertains to a C₁₋₂₀ alkyl group in which at least one hydrogen atom has been replaced with an aryl group. Examples of such groups include, but are not limited to, benzyl (phenylmethyl, PhCH₂—), benzhydryl (Ph₂CH—), trityl (triphenylmethyl, Ph₃C—), and phenethyl (phenylethyl, Ph-CH₂CH₂—). Typically a substituted alkyl group carries 1, 2 or 3 substituents, for instance 1 or 2.

The term “alkenyl”, as used herein, refers to a linear or branched chain hydrocarbon radical comprising one or more double bonds. An alkenyl group may be a C₂₋₂₀ alkenyl group, a C₂₋₁₀ alkenyl group or a C₂₋₆ alkenyl group. Examples of C₂₋₂₀ alkenyl groups include those related to C₂₋₂₀ alkyl groups by the insertion of one or more double bonds. Alkenyl groups typically comprise one or two double bonds. The alkenyl groups referred to herein may be substituted or unsubstituted, as defined for alkyl groups above.

The term “alkynyl”, as used herein, refers to a linear or branched chain hydrocarbon radical comprising one or more triple bonds. An alkynyl group may be a C₂₋₂₀ alkynyl group, a C₂₋₁₀ alkynyl group a C₂₋₆ alkynyl group. Examples of C₂₋₂₀ alkynyl groups include those related to C₂₋₂₀ alkyl groups by the insertion of one or more triple bonds. Alkynyl groups typically comprise one or two triple bonds. The alkynyl groups referred to herein may be substituted or unsubstituted, as defined for alkyl groups above.

The term “cycloalkyl group”, as used herein, refers to a substituted or unsubstituted alkyl group which is also a cyclyl group; that is, a monovalent moiety obtained by removing a hydrogen atom from an alicyclic ring atom of a carbocyclic ring of a carbocyclic compound. A cycloalkyl group may have from 3 to 25 carbon atoms (unless otherwise specified), including from 3 to 25 ring atoms. Thus, the term “cycloalkyl” includes the sub-classes cycloalkyenyl and cycloalkynyl. Examples of groups of C₃₋₂₅ cycloalkyl groups include C₃₋₂₀ cycloalkyl, C₃₋₁₅ cycloalkyl, C₃₋₁₀ cycloalkyl, and C₃₋₂₅ cycloalkyl. When a C₃₋₂₅ cycloalkyl group is substituted it typically bears one or more substituents selected from substituted or unsubstituted C₁₋₆ alkyl, substituted or unsubstituted aryl, substituted or unsubstituted heteroaryl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted cycloalkyl group carries 1, 2 or 3 substituents, for instance 1 or 2.

Examples of C3-25 cycloalkyl groups include, but are not limited to, those derived from saturated monocyclic hydrocarbon compounds, which C₃₋₂₅ cycloalkyl groups are substituted or unsubstituted as defined above: cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀); unsaturated monocyclic hydrocarbon compounds: cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈); saturated polycyclic hydrocarbon compounds: thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀); unsaturated polycyclic hydrocarbon compounds: camphene (C₁₀), limonene (C₁₀), pinene (C₁₀); polycyclic hydrocarbon compounds having an aromatic ring: indene (C₉), indane (e.g., 2,3-dihydro-1H-indene) (C9), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

The term “heterocyclyl group”, as used herein, refers to a substituted or unsubstituted monovalent moiety obtained by removing a hydrogen atom from a ring atom of a heterocyclic compound, which moiety has from 3 to 20 ring atoms (unless otherwise specified), of which from 1 to 10 are ring heteroatoms. Heterocyclic compounds include aromatic heterocyclic compounds and non-aromatic heterocyclic compounds. Preferably, each ring has from 3 to 7 ring atoms, of which from 1 to 4 are ring heteroatoms. When a C₃₋₂₀ heterocyclyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₂₀ heterocyclyl group carries 1, 2 or 3 substituents, for instance 1 or 2.

Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of (non-aromatic) monocyclic C₃₋₂₀ heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (CQ).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of C₃₋₂₀ heterocyclyl groups which are also aryl groups are described below as heteroaryl groups.

The term “aryl group”, as used herein, refers to a substituted or unsubstituted, monocyclic or polycyclic (for instance bicyclic) aromatic group which typically contains from 6 to 14 carbon atoms, preferably from 6 to 10 carbon atoms in the ring portion. Examples include phenyl, naphthyl, indenyl, indanyl, anthracenyl and pyrenyl groups. An aryl group is substituted or unsubstituted. When an aryl group is substituted it typically bears one or more substituents selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Typically it carries 0, 1, 2 or 3 substituents. A substituted aryl group may be substituted in two positions with a single C₁₋₆ alkylene group, or with a bidentate group represented by the formula —X—C₁₋₆ alkylene, or —X—C₁₋₆ alkylene-X—, wherein X is selected from O, S and NR, and wherein R is H, aryl or C₁₋₆ alkyl. Thus a substituted aryl group may be an aryl group fused with a cycloalkyl group or with a heterocyclyl group. The term “aralkyl” as used herein, pertains to an aryl group in which at least one hydrogen atom (e.g., 1, 2, 3) has been substituted with a C₁₋₆ alkyl group. Examples of such groups include, but are not limited to, tolyl (from toluene), xylyl (from xylene), mesityl (from mesitylene), and cumenyl (or cumyl, from cumene), and duryl (from durene).

The ring atoms of an aryl group may include one or more heteroatoms (as in a heteroaryl group). Such an aryl group is a heteroaryl group, and is a substituted or unsubstituted monocyclic or polycyclic (for instance bicyclic) heteroaromatic group which typically contains from 6 to 14 atoms, for instance 6 to 10 atoms, in the ring portion including one or more heteroatoms. It is generally a 5- or 6-membered ring, containing at least one heteroatom selected from O, S, N, P, Se and Si. It may contain, for example, 1, 2 or 3 heteroatoms. Examples of heteroaryl groups include pyridyl, pyrazinyl, pyrimidinyl, pyridazinyl, furanyl, thienyl, pyrazolidinyl, pyrrolyl, oxazolyl, oxadiazolyl, isoxazolyl, thiadiazolyl, thiazolyl, isothiazolyl, imidazolyl, pyrazolyl, triazolyl, indolyl, benzimidazolyl, indazolyl, benzotriazolyl, pyrrolopyridinyl, pyrrolopyrimidinyl, purinyl, indolizinyl, pyrrolopyrazinyl, pyrrolopyriminyl, pyrrolopyridazinyl, imidazopyridinyl, pyrazolopyridinyl, imidazopyridazinyl, imidazopyrimidinyl, imidazopyrazinyl, imidazopyrimidinyl, triazolopyridinyl, quinolyl, isoquinolyl, cinnolinyl, quinazolinyl, quinoxalinyl, phthalazinyl, pyridopyrazinyl, pteridinyl, pyridopyridazinyl, naphthyridinyl, and carbazolyl. A heteroaryl group may be substituted or unsubstituted, for instance, as specified above for aryl. Typically it carries 0, 1, 2 or 3 substituents.

The term “alkylene group” as used herein, refers to an substituted or unsubstituted bidentate moiety obtained by removing two hydrogen atoms, either both from the same carbon atom, or one from each of two different carbon atoms, of a hydrocarbon compound having from 1 to 20 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below. Typically it is C₁₋₁₀ alkylene, for instance C₁₋₆ alkylene. Typically it is C₁₋₄ alkylene, for example methylene, ethylene, i-propylene, n-propylene, t-butylene, s-butylene or n-butylene. It may also be pentylene, hexylene, heptylene, octylene and the various branched chain isomers thereof. An alkylene group may be substituted or unsubstituted, for instance, as specified above for alkyl. Typically a substituted alkylene group carries 1, 2 or 3 substituents, for instance 1 or 2.

In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkylene,” as used herein, pertains to an alkylene group having from 1 to 4 carbon atoms. Examples of groups of alkylene groups include C₁₋₄ alkylene (“lower alkylene”), C₁₋₇ alkylene, C₁₋₁₀ alkylene and C₁₋₂₀ alkylene.

Examples of linear saturated C₁₋₇ alkylene groups include, but are not limited to, —(CH₂)ₙ— where n is an integer from 1 to 7, for example, —CH₂— (methylene), —CH₂CH₂— (ethylene), —CH₂CH₂CH₂— (propylene), and —CH₂CH₂CH₂CH₂— (butylene).

Examples of branched saturated C₁₋₇ alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃) CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₁₋₇ alkylene groups include, but are not limited to, —CH═CH— (vinylene), —CH═CH—CH₂—, —CH₂—CH═CH₂—, —CH═CH—CH₂—CH₂—, —CH═CH—CH₂—CH₂—CH₂—, —CH═CH—CH═CH—, —CH—CH—CH—CH—CH₂—, —CH—CH—CH—CH—CH₂—CH₂—, —CH—CH—CH₂—CH—CH—, and —CH═CH—CH₂—CH₂—CH═CH—.

Examples of branched partially unsaturated C₁₋₇ alkylene groups include, but are not limited to, —C(CH₃)═CH—, —C(CH₃)═CH—CH₂—, and —CH═CH—CH(CH₃)—.

Partially unsaturated alkylene groups comprising one or more double bonds may be referred to as alkenylene groups. Partially unsaturated alkylene groups comprising one or more triple bonds may be referred to as alkynylene groups (for instance —C≡C—, CH₂—C≡C—, and —CH2-C≡C≡CH₂—).

Examples of alicyclic saturated C₁₋₇ alkylene groups include, but are not limited to, cyclopentylene (e.g., cyclopent-1,3-ylene), and cyclohexylene (e.g., cyclohex-1,4-ylene). Examples of alicyclic partially unsaturated C₁₋₇ alkylene groups include, but are not limited to, cyclopentenylene (e.g., 4-cyclopenten-1,3-ylene), cyclohexenylene (e.g., 2-cyclohexen-1,4-ylene; 3-cyclohexen-1,2-ylene; 2,5-cyclohexadien-1,4-ylene).

As used herein the term “oxo” represents a group of formula: ═O.

As used herein the term “acyl” represents a group of formula: —C(═O)R, wherein R is an acyl substituent, for example, a substituted or unsubstituted C₁₋₂₀ alkyl group, substituted or unsubstituted C₂₋₂₀ alkenyl group, substituted or unsubstituted C₂₋₂₀ alkynyl group, a substituted or unsubstituted C₃₋₂₀ heterocyclyl group, a substituted or unsubstituted aryl group or a substituted or unsubstituted heteroaryl group, for instance a substituted or unsubstituted C₁₋₆alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

As used herein the term “acyloxy” (or reverse ester) represents a group of formula: —OC(═O)R, wherein R is an acyloxy substituent, for example, a substituted or unsubstituted C₁₋₂₀ alkyl group, substituted or unsubstituted C₂₋₂₀ alkenyl group, substituted or unsubstituted C₂₋₂₀ alkynyl group, a substituted or unsubstituted C₃₋₂₀ heterocyclyl group, a substituted or unsubstituted aryl group or a substituted or unsubstituted heteroaryl group, for instance a substituted or unsubstituted C₁₋₆ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

As used herein the term “ester” (or carboxylate, carboxylic acid ester or oxycarbonyl) represents a group of formula: —C(═O)OR, wherein R is an ester substituent, for example, a substituted or unsubstituted C₁₋₂₀ alkyl group, substituted or unsubstituted C₂₋₂₀ alkenyl group, substituted or unsubstituted C₂₋₂₀ alkynyl group, a substituted or unsubstituted C₃₋₂₀ heterocyclyl group, a substituted or unsubstituted aryl group or a substituted or unsubstituted heteroaryl group, for instance a substituted or unsubstituted C1.6 alkyl group. Examples of ester groups include, but are not limited to, —C(═O)OCH₃, —C(═O)OCH₂CH₃, —C(═O)OC(CH₃)₃, and —C(═O)OPh.

As used herein the term “amino” represents a group of formula —NH₂. The term “C₁-C₁₀ alkylamino” represents a group of formula —NHR′ wherein R′ is a C₁₋₁₀ alkyl group, preferably a C₁₋₆ alkyl group, as defined previously. The term “di(C₁₋₁₀)alkylamino” represents a group of formula —NR′R″ wherein R′ and R″ are the same or different and represent C₁₋₁₀ alkyl groups, preferably C₁₋₆ alkyl groups, as defined previously. The term “arylamino” represents a group of formula —NHR′ wherein R′ is an aryl group, preferably a phenyl group, as defined previously. The term “diarylamino” represents a group of formula —NR′R″ wherein R′ and R″ are the same or different and represent aryl groups, preferably phenyl groups, as defined previously. The term “arylalkylamino” represents a group of formula —NR′R″ wherein R′ is a C₁₋₁₀ alkyl group, preferably a C₁₋₆ alkyl group, and R″ is an aryl group, preferably a phenyl group.

A halo group is chlorine, fluorine, bromine or iodine (a chloro group, a fluoro group, a bromo group or an iodo group). It is typically chlorine, fluorine or bromine.

The term “halide”, as used herein, refers to fluoride, chloride, bromide and iodide.

As used herein the term “amido” represents a group of formula: —C(═O)NR′R″, wherein R′ and R″ are independently amino substituents, as defined for di(C₁₋₁₀)alkylamino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′ and R″, together with the nitrogen atom to which they are attached, form a heterocyclic structure as in, for example, piperidinocarbonyl, morpholinocarbonyl, thiomorpholinocarbonyl, and piperazinocarbonyl.

As used herein the term “acylamido” represents a group of formula: —NR′C(—O)R″, wherein R′ is an amide substituent, for example, hydrogen, a C₁₋₂₀ alkyl group, a C₃₋₂₀ heterocyclyl group, an aryl group, preferably hydrogen or a C₁₋₂₀ alkyl group, and R″ is an acyl substituent, for example, a C₁₋₂₀ alkyl group, a C₃₋₂₀ heterocyclyl group, or an aryl group, preferably hydrogen or a C₁₋₂₀ alkyl group. Examples of acylamide groups include, but are not limited to, —NHC(═O)CH₃, —NHC(═O)CH₂CH₃, —NHC(═O)Ph, —NHC(═O)C₁₅H₃₁ and —NHC(═O)C₉H₁₉. Thus, a substituted C₁₋₂₀ alkyl group may comprise an acylamido substituent defined by the formula —NHC(═O)—C₁₋₂₀ alkyl, such as —NHC(═O)C₁₅H₃₁ or —NHC(═O)C₉H₁₉. R¹ and R² may together form a cyclic structure, as in, for example, succinimidyl, maleimidyl, and phthalimidyl:

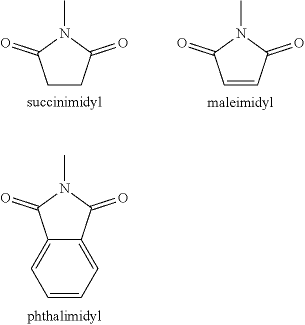

A C₁₋₁₀ alkylthio group is a said C₁₋₁₀ alkyl group, preferably a C₁₋₆ alkyl group, attached to a thio group. An arylthio group is an aryl group, preferably a phenyl group, attached to a thio group.

A C₁₋₂₀ alkoxy group is a said substituted or unsubstituted C₁₋₂₀ alkyl group attached to an oxygen atom. A C₁₋₆ alkoxy group is a said substituted or unsubstituted C₁₋₆ alkyl group attached to an oxygen atom. A C₁₋₄ alkoxy group is a substituted or unsubstituted C₁₋₄ alkyl group attached to an oxygen atom. Examples of C₁₋₄ alkoxy groups include, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy). Further examples of C₁₋₂₀ alkoxy groups are —O(Adamantyl), —O—CH₂-Adamantyl and —O—CH₂—CH₂-Adamantyl.

An aryloxy group is a substituted or unsubstituted aryl group, as defined herein, attached to an oxygen atom. An example of an aryloxy group is —OPh (phenoxy).

Unless otherwise specified, included in the above are the well known ionic, salt, solvate, and protected forms of these substituents. For example, a reference to carboxylic acid or carboxyl group (—COOH) also includes the anionic (carboxylate) form (—COO⁻), a salt or solvate thereof, as well as conventional protected forms. Similarly, a reference to an amino group includes the protonated form (—N⁺HR′R″), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Certain compounds may exist in one or more particular geometric, optical, enantiomeric, diastereomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto, enol, and enolate forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like, unless otherwise specified. However, reference to an isotope of fluorine refers only to that isotope of fluorine. In particular, reference to ¹⁸F includes only ¹⁸F. Reference to fluorine without specifying the isotope may refer to ¹⁸F or ¹⁹F depending on context. Typically, reference to “F” (i.e. without defining the isotope) refers to the ¹⁹F, i.e. stable fluorine.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting known methods, in a known manner.

The term “substituted”, as used herein, may be as defined above for particular groups. However, in some instances, the term substituted may refer to a group substituted with a group selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted aryl, substituted or unsubstituted heteroaryl, substituted or unsubstituted cycloalkyl, substituted or unsubstituted heterocyclyl, cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. In other instances, the term “substituted” may refer to a group substituted with a group selected from substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted cycloalkyl, substituted or unsubstituted heterocyclyl, cyano, amino, C₁₋₆ alkylamino, di(C₁₋₆)alkylamino, arylamino, diarylamino, arylalkylamino, amido, acylamido, hydroxy, oxo, halo, carboxy, ester, acyl, acyloxy, C₁₋₆ alkoxy, aryloxy, haloalkyl, sulfonic acid, thiol, C₁₋₆ alkylthio, arylthio, sulfonyl, phosphoric acid, phosphate ester, phosphonic acid and phosphonate ester. For example, the term “substituted” may refer to a group substituted with a group selected unsubstituted C₁₋₆ alkyl, unsubstituted C₂₋₆ alkenyl, unsubstituted C₂₋₆ alkynyl, unsubstituted aryl, unsubstituted heteroaryl, cyano, amino, unsubstituted C₁₋₆ alkylamino, unsubstituted di(C₁₋₆)alkylamino, unsubstituted arylamino, unsubstituted diarylamino, unsubstituted arylalkylamino, unsubstituted amido, unsubstituted acylamido, hydroxy, oxo, halo, carboxy, unsubstituted ester, unsubstituted acyl, unsubstituted acyloxy, unsubstituted C₁₋₆ alkoxy, unsubstituted aryloxy, sulfonic acid, thiol, unsubstituted C₁₋₆ alkylthio, unsubstituted arylthio, sulfonyl, phosphoric acid, unsubstituted phosphate ester, unsubstituted phosphonic acid and unsubstituted phosphonate ester.

The term “¹⁸F” refers to an atom of the specific isotope of fluorine having 9 protons and 9 neutrons. The terms “¹⁸F⁻” and “¹⁸F-fluoride” refer to an anion of the atom of the specific isotope of fluorine having 9 protons and 9 neutrons.

The use of “¹⁸F-” before a chemical entity name or “¹⁸F[chemical formula]” refers to a chemical entity in which a ¹⁹F has been replaced with an ¹⁸F. Therefore, the terms “¹⁸F-trifluoromethyl” and “¹⁸F[CF₃]” as used herein refer to a —CF₃ (trifluoromethyl) group in which one of the three fluorines is ¹⁸F, i.e. a group of formula —CF₂¹⁸F.

The term “leaving group” as used herein refers to an atom or group, either charged or uncharged, that becomes detached from an atom in the residual or main part of the substrate in a particular reaction. The leaving group may or may not retain the bonding pair of electrons when it detaches from the atom in the residual or main part of the substrate. After detaching from the atom in the residual or main part of the substrate, the leaving group may have a positive charge, a negative charge or no charge (neutral charge).

The term “ligand”, as used herein, refers to a species capable of binding to a central atom to form a complex. Ligands may be charged or neutral species. Typically, as referred to herein, a ligand is a neutral species.

The term “transition metal” as used herein means any one of the three series of elements arising from the filling of the 3d, 4d and 5d shells, and situated in the periodic table following the alkaline earth metals. This definition is used in N. N. Greenwood and A. Earnshaw “Chemistry of the Elements”, First Edition 1984, Pergamon Press Ltd., at page 1060, first paragraph, with respect to the term “transition element”. The same definition is used herein for the term “transition metal”. Thus, the term “transition metal”, as used herein, includes all of Sc, Y, Ti, Zr, Hf, V, Nb, Ta, Cr, Mo, W, Mn, Tc, Re, Fe, Ru, Os, Co, Rh, Ir, Ni, Pd, Pt, Cu, Ag, Au, Zn, Cd and Hg. These are also referred to as the first, second and third row transition metals (i.e. the transition metals in periods 4, 5 and 6 of the periodic table).

The term “carbene” as used herein refers to electrically neutral species H₂C: and its derivatives, in which the carbon is covalently bonded to two univalent groups of any kind or a divalent group. The carbon of the carbene bears two nonbonding electrons, which may be spin-paired (singlet state) or spin-non-paired (triplet state). The term “difluorocarbene” as used herein refers to F₂C.

Process

The invention provides a process for producing a compound comprising the anion [CF₂¹⁸FSO₂]⁻, which process comprises treating a difluorocarbene source with
(i) a source of ¹⁸F⁻ and
(ii) a source of SO₂.

Typically the difluorocarbene source is treated with both (i) and (ii) simultaneously. Thus, typically the process comprises treating the difluorocarbene source with ¹⁸F⁻ in the presence of the source of SO₂. Equivalently, the process may comprise treating the difluorocarbene source with the source of SO₂ in the presence of ¹⁸F⁻. The source of ¹⁸F⁻ may be any suitable source of ¹⁸F⁻, as discussed below. Often ¹⁸F⁻ will be solvated.

Difluorocarbene Source

Any suitable difluorocarbene source may be used in the process of the invention. Typically, the difluorocarbene source provides difluorocarbene via an alpha elimination reaction. The alpha elimination reaction is usually a transformation of the type:

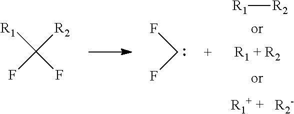

wherein R₁ is a first leaving group and R₂ is a second leaving group. The loss of R₁ and R₂ from the central CF₂ group generates difluorocarbene (:CF₂). Typically, the difluorocarbene generated is free difluorocarbene.

The difluorocarbene source may be a compound of Formula (I):

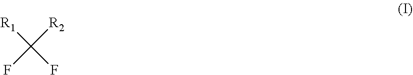

wherein R₁ is a first leaving group and R₂ is a second leaving group. R₁ generally retains the (CF₂—R₁) bonding electron pair when R₁ detaches from the central CF₂ moiety. R₂ usually does not retain the (CF₂—R₂) bonding electron pair when R₂ detaches from the central CF₂ moiety.

R₁ may comprise a positively charged functional group. R₂ may comprise a negatively charged functional group. One or both of R₁ and R₂ may have no charge (neutral charge). In the case where there is a net charge on the compound of Formula I, one or more counterions may be present. For instance, if the compound of Formula I is positively charged, one or more counter-anions may be present. The one or more counter-anions may be selected from any anion described herein. For instance, the one or more anions may be selected from halide, hydroxide, sulfate, and nitrate. Alternatively, the compound of Formula I may be anionic, and associated with one or more counter-cations. Again, any suitable counter-cation may be employed; many such cations are known to the skilled person.

In some instances, R₁ may comprise a positively charged functional group and R₂ may comprise a negatively charged functional group. In this case, there may be no net charge on the compound of Formula I. Thus, the compound of Formula I may be zwitterionic.

Examples of suitable R₁ groups include:
halo;
sulfonic esters, for instance groups having the formula —OS(O)₂R wherein R is halo, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl or substituted or unsubstituted heteroaryl, preferably wherein R is substituted or unsubstituted aryl. Examples of sulfonic esters include, but are not limited to triflate, mesylate and tosylate groups;
sulfones, for instance groups having the formula —S(O)₂R wherein R is halo, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl or substituted or unsubstituted heteroaryl;
groups having the formula —S(O)(NR″)R′ wherein R′ is halo, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl or substituted or unsubstituted heteroaryl, and wherein R″ is a sulfone, for instance a group having the formula —S(O)₂R as described above, preferably wherein R″ is a tosyl group;
ammonium cations, for instance ammonium cations having the formula —[NR₃R₄R₅]⁺, wherein R₃, R₄ and R₅ are each independently selected from H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl and substituted or unsubstituted heteroaryl; and wherein two or more of R₃, R₄ and R₅ may be bonded together to form one or more rings; and
phosphonium cations, for instance phosphonium cations having the formula —[PR₃R₄R₅]⁺, wherein R₃, R₄ and R₅ are each independently selected from H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl and substituted or unsubstituted heteroaryl; and wherein two or more of R₃, R₄ and R₅ may be bonded together to form one or more rings.

Examples of suitable R₂ groups include:
hydrogen;
carboxylate (—C(O)O⁻);
esters and carboxylic acids, for instance a group of formula —C(O)OR₉, wherein R₉ is hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl or substituted or unsubstituted heteroaryl, or wherein R₉ is a group of formula —Si(R₁₀R₁₁R₁₂) wherein R₁₀, R₁₁ and R₁₂ are each independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl, C₁₋₂₀ alkoxy, aryloxy and halo;
ketones, for instance groups of formula —C(O)R wherein R is substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl or substituted or unsubstituted heteroaryl;
silyl groups, for instance a group of formula —Si(R₁₀R₁₁R₁₂) wherein R₁₀, R₁₁ and R₁₂ are each independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl, C₁₋₂₀ alkoxy, aryloxy and halo,
stannyl groups, for instance a group of formula —Sn(R₁₀R₁₁R₁₂) wherein R₁₀, R₁₁ and R₁₂ are each independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl, C₁₋₂₀ alkoxy, aryloxy and halo;
sulfones, for instance groups having the formula —S(O)₂R wherein R is halo, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl or substituted or unsubstituted heteroaryl;
phosphonates, for instance groups having the formula —P(O)(OR)₂ wherein each R is independently selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl and substituted or unsubstituted heteroaryl;
sulfonium groups, for instance groups having the formula —[SR₂]⁺ wherein each R is independently selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl and substituted or unsubstituted heteroaryl, preferably wherein each R is substituted or unsubstituted aryl;
mercury (II) salts, for instance groups having the formula —HgX, wherein X is halide, hydroxide or alkoxide.

In some instances, R₁ and R₂, together with the CF₂ group to which they are attached may form a cyclic structure.

Examples of difluorocarbene sources are set out in the review by Ni et al. (Ni. C., Hu. J., Recent Advances in the Synthetic Application ofDifluorocarbene, SYNTHESIS, 2014, 46, 0842-0863). For instance, the difluorocarbene source may be CF₃H, HCF₂Cl, HCF₂Br, ClCF₂C(O)O⁻Na⁺, ClCF₂C(O)OMe, BrCF₂C(O)O⁻Na⁺, FSO₂CF₂COOH, FS(O)₂CF₂C(O)OMe, F₃CS(O)₂CF₂H, FS(O)₂CF₂C(O)OSi(CH₃)₃ (TFDA), ClF₂CSi(CH₃)₃, BrF₂CSi(CH₃)₃, F₃CSi(CH₃)₃, ClF₂CC(O)Ph, ClF₂CS(O)₂Ph, BrF₂CP(O)(OEt)₂, HF₂CS(O)(NTs)Ph, HF₂COTf, [PhArSCF₂Br]⁻+OTf⁻, [HF₂CN(nBu)₃]⁺Cl⁻, Ph₃P⁺CF₂C(O)O⁻, HgICF₃, (CH₃)₃SnCF₃ or tetrafluoroethane beta-sulfone derivatives.

Typically, R₁ is a phosphonium or an ammonium cation. For instance, R₁ may be a phosphonium cation of formula —[PR₃R₄R₅]⁺ or an ammonium cation of formula —[NR₃R₄R₅]⁺, wherein R₃, R₄ and R₅ are each independently selected from H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl and substituted or unsubstituted heteroaryl; and wherein two or more of R₃, R₄ and R₅ may be bonded together to form one or more rings.

Preferably, R₁ is a phosphonium cation of formula —[PR₃R₄R₅]+. For instance, R₁ may be a phosphonium cation of formula —[PR₃R₄R₅]⁺ wherein R₃, R₄ and R₅ are substituted or unsubstituted aryl. Thus, R₁ may be —[PPh₃]⁺.

Typically, R₂ is —C(O)O⁻, or a group of formula —C(O)OR₉, wherein R₉ is selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl and substituted or unsubstituted heteroaryl; or wherein R₉ is a group of formula —Si(R₁₀R₁₁R₁₂) wherein R₁₀, R₁₁ and R₁₂ are each independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl, C₁₋₂₀ alkoxy, aryloxy and halo. Preferably, R₂ is —C(O)O⁻.

Thus, in Formula I, the R₁ group may be a phosphonium or ammonium cation and the R₂ group may be —C(O)O⁻, or a group of formula —C(O)OR₉, wherein R₉ is selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl and substituted or unsubstituted heteroaryl; or wherein R₉ is a group of formula —Si(R₁₀R₁₁R₁₂) wherein R₁₀, R₁₁ and R₁₂ are each independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl, C₁₋₂₀ alkoxy, aryloxy and halo.

Preferably R₁ is a phosphonium cation of formula —[PR₃R₄R₅]⁺, wherein R₃, R₄ and R₅ are each independently selected from H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl and substituted or unsubstituted heteroaryl; and wherein two or more of R₃, R₄ and R₅ may be bonded together to form one or more rings, and R₂ is —C(O)O⁻.

Thus, in one embodiment the difluorocarbene source is (triphenylphosponio)difluoroacetate ([Ph₃P]⁺CF₂COO⁻).

The generation of difluorocarbene from the difluorocarbene source may require an additive to initiate difluorocarbene generation. Typically, the additive is a nucleophile or a base. Examples of nucleophiles include, but are not limited to halide or hydroxide. Examples of bases include, but are not limited to metal hydroxides, metal carbonates, metal hydrides, metal alkoxides and phosphinimine bases.

The difluorocarbene source may release difluorcarbene as a result of heating (i.e. via thermal pyrolysis). Typically, the difluorocarbene source is heated to a temperature from 80 to 150° C. For instance, the difluorocarbene source may be (triphenylphosponio)difluoroacetate (Ph₃P⁺CF₂C(O)O⁻) and the process of the invention may comprise heating the difluorocarbene source to a temperature of from 80 to 150° C. to generate difluorcarbene.

Source of SO₂

Any suitable source of SO₂ may be used in the process of the invention. The source of SO₂ may be free SO₂ which is a gas at room temperature, or a compound that provides SO₂ in situ. Typically, the source of SO₂ is a compound that provides SO₂ in situ. Typically the compound that provides SO₂ in situ comprises a heteroatom-SO₂ bond, for instance an N—SO₂ bond.

Thus, the source of SO₂ may be a compound of Formula (II):

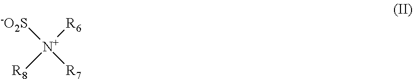

wherein R₆, R₇ and R₈ are each independently selected from H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, and substituted or unsubstituted heteroaryl;
provided that when at least two of R₆, R₇ and R₈ are substituted or unsubstituted C₁₋₂₀ alkyl groups, two of said substituted or unsubstituted C₁₋₂₀ alkyl groups may be bonded to a single heteroatom to form a ring, optionally wherein the heteroatom is O, S or N, wherein said N may be part of a group NR^{y} or N⁺R^{y}R^{z} wherein R^{y} is H, C₁₋₆ alkyl or aryl, and R^{z} is SO₂⁻, and
provided that when all three of R₆, R₇ and R₈ are substituted or unsubstituted C₁₋₂₀ alkyl groups, all three of said substituted or unsubstituted C₁₋₂₀ alkyl groups may be bonded to a single heteroatom, N, wherein said N may be part of a group N⁺R^{z} wherein R^{z} is H, C₁₋₆ alkyl, aryl or SO₂⁻, and preferably wherein R^{z} is SO₂⁻.

Typically, R₆ is selected from substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, and substituted or unsubstituted heteroaryl; and R₇ and R₈ are substituted or unsubstituted C₁₋₂₀ alkyl groups which are bonded to a single heteroatom to form a ring, optionally wherein the heteroatom is O, S or N, wherein said N may be part of a group NR^{y} or N⁺R^{y}R^{z} wherein R^{y} is H, C₁₋₆ alkyl or aryl, and R is SO₂. Often R₆ is substituted or unsubstituted C₁₋₂₀ alkyl. Usually the heteroatom is O.

In one embodiment, the source of SO₂ is a compound of Formula (III):

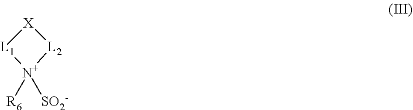

wherein X is selected from O, S, CH₂ and NH; L₁ and L₂ are substituted or unsubstituted C₁₋₆ alkylene, typically substituted or unsubstituted C₂₋₆ alkylene; and R₆ is substituted or unsubstituted C₁₋₂₀ alkyl. Often, X is O, L₁ and L₂ are unsubstituted C₁₋₆ alkylene and R₆ is unsubstituted C₁₋₆ alkyl. Thus, the source of SO₂ may be N-methylmorpholine-SO₂.

In one embodiment, the source of SO₂ is a compound of Formula (IV):

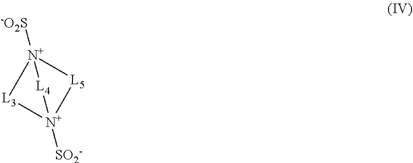

wherein L₃, L₄ and L₅ are selected from substituted or unsubstituted C₁₋₆ alkylene, preferably substituted or unsubstituted C₂₋₆ alkylene. Thus, the compound of Formula (IV) may be 1,4-diazabicyclo[2.2.2]octane bis(sulfur dioxide), also known as DABSO.

Compounds of Formulae II, III and IV advantageously act as sources of SO₂ without the need for employing toxic gaseous reagents. Further, N-methylmorpholine-SO₂ is particularly advantageous because the side products when this reagent is used are volatile and easy to separate out from the reaction mixture following reaction, for instance during purification.

¹⁸F⁻ Source

The ¹⁸F⁻ used in the process of the invention may be in any suitable form. Typically, the ¹⁸F⁻ is present as a salt. Thus, the source of ¹⁸F⁻ may be, or may comprise, a salt of ¹⁸F⁻. Thus, the process of the invention may comprise treating the difluorocarbene source with (i) a salt of ¹⁸F⁻ and (ii) the source of SO₂. Typically the concentration of ¹⁸F⁻ is less than or equal to 10⁻⁴ M, for instance less than or equal to 10⁻⁵ M. In some cases, the concentration of ¹⁸F⁻ will be nanomolar or less, for instance less than or equal to 10⁻⁸ M. ¹⁹F⁻ may also be present. In such a case, the total fluoride concentration (including ¹⁸F⁻ and ¹⁹F⁻) may be less than or equal to 10⁻⁴ M, for instance less than or equal to 10⁻⁵ M.

Any suitable source of ¹⁸F⁻ may be used. As will be understood by the skilled person the ¹⁸F⁻ will typically be present in the form of a salt, with a counter cation. Any suitable counter cation may be used. Typically, the counter cation is a quaternary ammonium cation, for instance tetrabutylammonium, or an alkali metal cation, for instance Cs⁺ or K⁺, or a proton, H⁺. Preferably, the source of ¹⁸F⁻ comprises an alkali metal or ammonium salt of ¹⁸F⁻.

When an alkali metal cation is employed, the alkali metal cation may be complexed in a cryptand, for instance aminopolyether 2.2.2 (K₂₂₂), which is commercially available as Kryptofix-222. Thus, the source of ¹⁸F⁻ may further comprise a cryptand ligand. Advantageously, the addition of such a cryptand enables the fluoride ion ¹⁸F⁻ to be solubilized in a polar aprotic solvent, for instance acetonitrile or DMF. It also enables the formation of a ‘naked fluoride ion’ as a KF-K₂₂₂ complex. In one embodiment, therefore, the source of ¹⁸F⁻ is a K[¹⁸F]F-K₂₂₂ complex. Alternatively, the source of ¹⁸F⁻ may be [¹⁸F]TEAF (tetraethylammonium fluoride), [¹⁸F]TBAF (tetrabutylammonium fluoride), [¹⁸F]CsF, or [¹⁸F]HF. Typically, ¹⁸F⁻ is present as K[₁₈F]F-K₂₂₂ or [¹⁸F]HF. More typically, ¹⁸F⁻ is present as K[¹⁸F]F-K₂₂₂.

The process of the present invention typically comprises treating the difluorocarbene source with at least 2 GBq of the ¹⁸F⁻.

Process—Further Details

Typically, the compound comprising [CF₂¹⁸FSO₂]⁻ further comprises a counter-cation. Thus, the process of the present invention may be a process for producing a compound of formula [CF₂¹⁸FSO₂]⁻ₙAⁿ⁺, wherein n is an integer of from 1 to 4.

Typically n is 1 or 2. Preferably, n is 1. When n is 1, Aⁿ⁺ may, for instance, be an alkali metal cation or an ammonium cation. Thus, Aⁿ⁺ may be Li⁺, Na⁺, K⁺, Rb⁺, Cs⁺ or Fr⁺. Ammonium cations include groups of formula [NRₐR_{b}R_{c}R_{d}]⁺, wherein Rₐ, R_{b}, R_{c} and R_{d} are each independently selected from H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl; and wherein two or more of Rₐ, R_{b}, R_{c} and R_{d} may be bonded together to form one or more rings. Typically, Rₐ, R_{b}, R_{c} and R_{d} are each independently selected from hydrogen and unsubstituted C₁₋₂₀ alkyl. Preferably, the ammonium cation is [NH₄]⁺. Therefore, the process may be a process for producing CF₂¹⁸FSO₂NH₄.

When n is 2, Aⁿ⁺ may be a divalent metal cation. For instance, A may be a divalent metal cation selected from alkaline earth metal cations and divalent transition metal cations. Such divalent metal cations include, but are not limited to Mg²⁺, Ca²⁺, Sr²⁺, Ba²⁺, Sc²⁺, Ti²⁺, V²⁺, Cr²⁺, Mn²⁺, Fe₂₊, Ni²⁺, Cu²⁺ or Zn²⁺.

In one embodiment of the process, the step of treating the difluorocarbene source with the source of ¹⁸F⁻ and the source of SO₂ is performed in the presence of Aⁿ⁺. In this embodiment, the source of ¹⁸F⁻ may comprise a metal salt of ¹⁸F⁻. The source of ¹⁸F⁻ may comprise a metal salt of ¹⁸F⁻, wherein Aₙ₊ is said metal. Preferably, the source of ¹⁸F⁻ comprises an alkali metal salt of ¹⁸F⁻, wherein Aⁿ⁺ is said alkali metal. Therefore, the step of treating the difluorocarbene source with the source of ¹⁸F⁻ and the source of SO₂ may be performed in the presence of Aⁿ⁺, wherein the source of ¹⁸F⁻ comprises an A salt of ¹⁸F⁻, preferably wherein Aⁿ⁺ is an alkali metal. Therefore, the process may be a process for producing an alkali metal salt of [CF₂¹⁸FSO₂], for instance CF₂¹⁸FSO₂K.

In another embodiment of the process, the step of treating the difluorocarbene source with the source of ¹⁸F⁻ and the source of SO₂ is performed in the presence of a first cation B^{m+} to produce a compound of formula [CF₂¹⁸FSO₂]⁻ₘB^{m+}, wherein m is an integer of from 1 to 4, and the process further comprises replacing the first cation B^{m+} with a different cation Aⁿ⁺, to produce said compound of formula [CF₂¹⁸FSO₂]⁻ₙAⁿ⁺.

In this embodiment, the source of ¹⁸F⁻ may comprise a metal salt of ¹⁸F⁻, wherein B^{m+} is said metal. Preferably the source of ¹⁸F⁻ comprises an alkali metal salt of ¹⁸F⁻, wherein B^{m+} is said alkali metal. Thus, B^{m+} may be Li⁺, Na⁺, K⁺, Rb⁺, Cs⁺ or Fr⁺. The compound of formula [CF₂¹⁸FSO₂]⁻ₘB^{m+} is often CF₂¹⁸FSO₂K.

In this embodiment, Aⁿ⁺ may be a non-metal cation. Preferably, Aⁿ⁺ is an ammonium cation. Typically, Aⁿ⁺ is an ammonium cation of formula [NRₐR_{b}R_{c}R_{d}]⁺, wherein Rₐ, R_{b}, R_{c} and R_{d} are each independently selected from H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl; and wherein two or more of Rₐ, R_{b}, R_{c} and R_{d} may be bonded together to form one or more rings. Typically, Rₐ, R_{b}, R_{c} and R_{d} are each independently selected from hydrogen and unsubstituted C₁₋₂₀ alkyl. Preferably, Aⁿ⁺ is [NH₄]⁺. Therefore, the compound of formula [CF₂¹⁸FSO₂]⁻ₙAⁿ⁺ may be CF₂¹⁸FSO₂NH₄.

In this embodiment, the step of replacing the first cation B^{m+} with a different cation Aⁿ⁺ to produce said compound of formula [CF₂¹⁸FSO₂⁻]ₙAⁿ⁺ may be a purification step. The cation Aⁿ⁺ may be a cation present in an elution buffer in said purification step. The purification step may be any purification step as described herein, for example the purification step may comprise (i) weak anion exchange chromatography, (ii) chromatography using a mixed-mode, strong anion-exchange cartridge (MAX cartridge) or (iii) reverse phase high performance liquid chromatography (HPLC).

The process of the invention is typically carried out in solution. The process may be carried out in solution in any suitable solvent. Typically the solvent is an aprotic solvent. For instance, the process may be carried out in the presence of a polar aprotic solvent. Typically, the process of the invention is carried out in the presence of one or more aprotic solvents, for instance one or more polar aprotic solvents.

Polar aprotic solvents are well known to the skilled person. The process may be carried out in the presence of a solvent selected from dimethyl formamide, propylene carbonate, N,N-dimethyl acetamide and acetonitrile. The one or more polar aprotic solvents may for instance be selected from dimethylformamide, propylene carbonate and mixtures thereof. For instance, the process may be carried out in a mixture of solvents, such as a mixture of dimethyl formamide and propylene carbonate.

The process may be performed under any suitable atmosphere. For instance, the process may be performed under an inert atmosphere such as nitrogen or argon, or the process may be performed in the presence of oxygen, for instance in air. Often, the process is performed in air.

The amount of the difluorocarbene source may be any suitable amount. The ratio of the amount of the difluorocarbene source to the amount of the SO₂ source may be from 1:40 to 40:1, for instance from 1:20 to 20:1. Typically, the amount of SO₂ source is less than the amount of difluorocarbene source. The molar ratio of the amount of the difluorocarbene source to the amount of the SO₂ source may be from 1:1 to 20:1, preferably 1:1 to 10:1, more preferably 1:1 to 5:1.

The amount of the source of ¹⁸F⁻ may be any suitable amount. Typically, the difluorocarbene source is treated with at least 2 GBq of the ¹⁸F⁻. The difluorocarbene source may be treated with at least 3 GBq of the ¹⁸F⁻, at least 4 GBq of the ¹⁸F⁻, at least 5 GBq of the ¹⁸F⁻, at least 6 GBq of the ¹⁸F⁻, at least 7 GBq of the ¹⁸F⁻, at least 8 GBq of the ¹⁸F⁻, at least 9 GBq of the ¹⁸F⁻ or at least 10 GBq of the ¹⁸F⁻.

In some embodiments, the difluorocarbene source, the ¹⁸F⁻ source and the SO₂ source are heated to a temperature of greater than room temperature, for instance to a temperature of at least 50° C., or a temperature of at least 70° C., for example a temperature of at least 80° C., such as, for instance, a temperature of from 80 to 150° C. Such a temperature may be used to induce thermal pyrolysis of the difluorocarbene source to provide difluorocarbene. For instance, the difluorocarbene source may be (triphenylphosponio)difluoroacetate and the difluorocarbene source, the ¹⁸F⁻ source and the SO₂ source may be heated to a temperature of from 80 to 150° C. The difluorocarbene source, the ¹⁸F⁻ source and the SO₂ source may be heated to a temperature of from 80 to 130° C., or a temperature of from 90 to 120° C., or a temperature of from 100 to 120° C.

The process of the present invention may further comprise a step of purifying the compound comprising the anion [CF₂¹⁸FSO₂]⁻. Any suitable purification method may be employed; a wide range of suitable purification methods is well known to the skilled person. The step of purifying may comprise performing chromatography, for example weak anion exchange chromatography, high-performance liquid chromatography, reverse-phase high-performance liquid chromatography. The step of purifying may comprise performing several purification methods to obtain the compound comprising the anion [CF₂^{1S}FSO₂]⁻.

In one embodiment, the process of the present invention is a process for producing a compound comprising the anion [CF₂¹⁸FSO₂]⁻, which process comprises treating (triphenylphosponio)difluoroacetate ([Ph₃P]⁺CF₂COO⁻) with (i) [¹⁸F]KF/K₂₂₂ and (ii)N-methylmorpholine-SO₂. Typically, the process is carried out at a temperature of from 80 to 150° C. Typically, the process is carried in out in a solution comprising a mixture of dimethyl formamide and propylene carbonate. Typically, this process produces CF₂¹⁸FSO₂K. The process may comprise further purification steps that result in a final product that is CF₂¹⁸FSO₂NH₄.

Compound comprising [CF₂¹⁸FSO₂]

The invention also provides a compound comprising the anion [CF₂¹⁸FSO₂]⁻. Typically, the compound comprises a counter cation. Any suitable counter-cation may be employed; many such cations are known to the skilled person. Thus, the compound comprising the anion may be [CF₂¹⁸FSO₂]⁻ₙAⁿ⁺, wherein n is an integer of from 1 to 4. Preferably n is 1 or 2. In a preferred embodiment, n is 1.

Typically, the compound of the invention comprises at least 500 MBq of the anion [CF₂¹⁸FSO₂]⁻. The compound of the invention may, for instance, comprise at least 600 MBq of the anion [CF₂¹⁸FSO₂]⁻, or for instance at least 700 MBq of the anion, of rexample at least 800 MBq, at least 900 MBq, or at least 1 GBq of the anion.

The invention further provides a composition comprising the compound of the invention, wherein the composition comprises at least any of the above-mentioned amounts of the anion [CF₂¹⁸FSO₂]⁻, in MBq or GBq.

In the compound of the invention, when n is 1, Aⁿ⁺ is typically an alkali metal cation or an ammonium cation. Aⁿ⁺ may be Li⁺, Na⁺, K⁺, Rb⁺, Cs⁺ or Fr⁺. Therefore the compound comprising the anion [CF₂⁸FSO₂]⁻ may be CF₂¹⁸FSO₂Li, CF₂¹⁸FSO₂Na, CF₂¹⁸FSO₂K, CF₂¹⁸FSO₂Rb, CF₂¹⁸FSO₂Cs or CF₂¹⁸FSO₂Fr. Often the compound is CF₂¹⁸FSO₂K.

Ammonium cations include groups of formula [NRₐR_{b}R_{c}R_{d}]⁺, wherein Rₐ, R_{b}, R_{c} and R_{d} are each independently selected from H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl; and wherein two or more of Rₐ, R_{b}, R_{c} and R_{d} may be bonded together to form one or more rings. Typically, Rₐ, R_{b}, R_{c} and R_{d} are each independently selected from hydrogen and unsubstituted C₁₋₂₀ alkyl. Typically, the ammonium cation is [NH₄]⁺, and the compound comprising the anion [CF₂¹⁸FSO₂]⁻ is CF₂¹⁸FSO₂NH₄.

When n is 2, Aⁿ⁺ is typically a divalent metal cation. Aⁿ⁺ may be a divalent metal cation selected from alkaline earth metal cations and divalent transition metal cations. For instance, Aⁿ⁺ may be Mg²⁺, Ca²⁺, Sr²⁺, Ba²⁺, Sc²⁺, Ti²⁺, V²⁺, Cr²⁺, Mn²⁺, Fe²⁺, Ni²⁺, Cu²⁺ or Zn²⁺. An may for instance be Zn²⁺.

The compound comprising the anion [CF₂¹⁸FSO₂] may be obtainable by the process of the invention as defined herein. The compound comprising the anion [CF₂¹⁸FSO₂] may be obtained by the process of the invention as defined herein. For instance, the compound comprising the anion [CF₂¹⁸FSO₂] may be obtained by a which process comprises treating a difluorocarbene source as defined anywhere herein with
(i) a source of ¹⁸F⁻ as defined anywhere herein and
(ii) a source of SO₂ as defined anywhere herein.

Process for Functionalising Aromatic Groups

The present invention also provides a process for producing a compound comprising an ¹⁸F-trifluoromethyl functionalised aromatic group, which process comprises contacting a compound comprising an aromatic group with a compound comprising the anion [CF₂¹⁸FSO₂]⁻ in the presence of an activator for trifluoromethyl radical formation.

Typically the compound comprising an ¹⁸F-trifluoromethyl functionalised aromatic group is treated with the compound comprising the anion [CF₂¹⁸FSO₂]⁻ and the activator for trifluoromethyl radical formation simultaneously. For instance, the process may comprise contacting a mixture comprising the compound comprising an aromatic group and the compound comprising the anion [CF₂¹⁸FSO₂]⁻ with the activator for trifluoromethyl radical formation. The process may comprise contacting a mixture comprising the compound comprising an aromatic group and activator for trifluoromethyl radical formation with the compound comprising the anion [CF₂¹⁸FSO₂]⁻. The process may comprise contacting a mixture comprising the activator for trifluoromethyl radical formation and the compound comprising the anion [CF₂¹⁸FSO₂]⁻ with the compound comprising an aromatic group.

An ¹⁸F-trifluoromethyl functionalised aromatic group corresponds to an aromatic group in which an ¹⁸F-trifluoromethyl group (—CF₂¹⁸F) has been bonded to an atom of the aromatic group. In the ¹⁸F-trifluoromethyl functionalised aromatic group the ¹⁸F-trifluoromethyl group replaces one of the substituents (either hydrogen or any other substituent as described herein, but typically hydrogen) on the aromatic group of the compound comprising an aromatic group.

Typically, the aromatic group is a substituted or unsubstituted aryl group or a substituted or unsubstituted heteroaryl group. When the aromatic group is a substituted or unsubstituted aryl group, the substituted or unsubstituted aryl group may be selected from substituted or unsubstituted phenyl, substituted or unsubstituted naphthyl, substituted or unsubstituted indenyl, substituted or unsubstituted indanyl, substituted or unsubstituted anthracenyl and substituted or unsubstituted pyrenyl. Typically the aromatic group is substituted or unsubstituted phenyl. Often, the aromatic group is an unsubstituted phenyl group or a phenol group.

When the aromatic group is a substituted or unsubstituted heteroaryl group, the substituted or unsubstituted heteroaryl group may be selected from substituted or unsubstituted pyridyl, substituted or unsubstituted pyrazinyl, substituted or unsubstituted pyrimidinyl, substituted or unsubstituted pyridazinyl, substituted or unsubstituted furanyl, substituted or unsubstituted thienyl, substituted or unsubstituted pyrazolidinyl, substituted or unsubstituted pyrrolyl, substituted or unsubstituted oxazolyl, substituted or unsubstituted oxadiazolyl, substituted or unsubstituted isoxazolyl, substituted or unsubstituted thiadiazolyl, substituted or unsubstituted thiazolyl, substituted or unsubstituted isothiazolyl, substituted or unsubstituted imidazolyl, substituted or unsubstituted pyrazolyl, substituted or unsubstituted triazolyl, substituted or unsubstituted indolyl, substituted or unsubstituted benzimidazolyl, substituted or unsubstituted indazolyl, substituted or unsubstituted benzotriazolyl, substituted or unsubstituted pyrrolopyridinyl, substituted or unsubstituted pyrrolopyrimidinyl, substituted or unsubstituted purinyl, substituted or unsubstituted indolizinyl, substituted or unsubstituted pyrrolopyrazinyl, substituted or unsubstituted pyrrolopyriminyl, substituted or unsubstituted pyrrolopyridazinyl, substituted or unsubstituted imidazopyridinyl, substituted or unsubstituted pyrazolopyridinyl, substituted or unsubstituted imidazopyridazinyl, substituted or unsubstituted imidazopyrimidinyl, substituted or unsubstituted imidazopyrazinyl, substituted or unsubstituted imidazopyrimidinyl, substituted or unsubstituted triazolopyridinyl, substituted or unsubstituted quinolyl, substituted or unsubstituted isoquinolyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyridopyridazinyl, substituted or unsubstituted naphthyridinyl, and substituted or unsubstituted carbazolyl. The aromatic group may be a substituted or unsubstituted indole group or a substituted or unsubstituted imidazole group. For instance, the aromatic group may be an unsubstituted indolyl group or an unsubstituted imidazolyl group.

The compound comprising an aromatic group may comprise at least one aromatic group, at least two aromatic groups or at least three aromatic groups as described herein. The compound comprising an aromatic group may have a single aromatic group as described herein. The compound comprising an aromatic group may have a two aromatic groups as described herein. The compound comprising an aromatic group may have a three aromatic groups as described herein. The process may comprise functionalising at least one of the aromatic groups on the compound comprising an aromatic group, at least two of the aromatic groups on the compound comprising an aromatic group or at least three of the aromatic groups on the compound comprising an aromatic group. When the compound comprising an aromatic group comprises more than one aromatic group, ¹⁸F-trifluoromethylation may occur preferentially at one of the aromatic groups. When the compound comprising an aromatic group comprises more than one aromatic group, ¹⁸F-trifluoromethylation may occur at all of the aromatic groups present.

The compound comprising an aromatic group may be an amino acid or a compound comprising an amino acid. Preferably, the amino acid is tyrosine, tryptophan, phenylalanine or histidine. More preferably the amino acid is tyrosine or tryptophan. In some embodiments the amino acid is tyrosine. In some embodiments the amino acid is tryptophan. Hence, the compound comprising an aromatic group may be tyrosine, tryptophan, phenylalanine or histidine or a compound comprising tyrosine, tryptophan, phenylalanine or histidine. Preferably, the compound comprising an aromatic group is tyrosine or tryptophan or a compound comprising tyrosine or tryptophan.

The compound comprising an aromatic group may be a peptide or a protein.

When the compound comprising an aromatic group is a peptide, the peptide may be a dipeptide, a tripeptide, an oligopeptide or a polypeptide. The peptide may be an alkaloid, an anti-microbial agent, a hormone, a growth factor, an immunomodulating agent or an anti-oxidant.

The compound comprising an aromatic group may be a peptide or protein comprising tyrosine, tryptophan, phenylalanine or histidine. Preferably, the compound comprising an aromatic group is a peptide or protein comprising tyrosine or tryptophan. The compound comprising an aromatic group may be a peptide or protein comprising tyrosine and tryptophan.

The peptide or protein may comprise at least one of tyrosine, tryptophan, phenylalanine or histidine, at least two of tyrosine, tryptophan, phenylalanine or histidine or at least three of tyrosine, tryptophan, phenylalanine or histidine. In some instances, the peptide or protein comprises at least one tyrosine residue and at least one tryptophan residue, for example Endomorphin I. In some instances, the peptide or protein comprises at least one tyrosine residue and at least one phenylalanine residue, for example insulin. In some instances, the peptide or protein comprises at least one tyrosine residue and at least one histidine residue, for example Angiotensin I/II or insulin. In some instances, the peptide or protein comprises at least one tryptophan residue and at least one phenylalanine residue, for example Somatostatin-14. In some instances, the peptide or protein comprises at least one tryptophan residue and at least one histidine residue. In some instances, the peptide or protein comprises at least one phenylalanine residue and at least one histidine residue, for example insulin.

The peptide or protein may comprise at least one tyrosine residue, at least two tyrosine residues or at least three tyrosine residues. The peptide or protein may comprise at least one tryptophan residue, at least two tryptophan residues or at least three tryptophan residues. The peptide or protein may comprise at least one phenylalanine residue, at least two phenylalanine residues or at least three phenylalanine residues. The peptide or protein may comprise at least one histidine residue, at least two histidine residues or at least three histidine residues.

The compound comprising an aromatic group may be a peptide selected from Thymogen, Endomorphin I, Melittin, Angiotensin I/II, Insulin, Somatostatin-14 and cyclo(-Arg-Gly-Asp-D-Tyr-Lys):

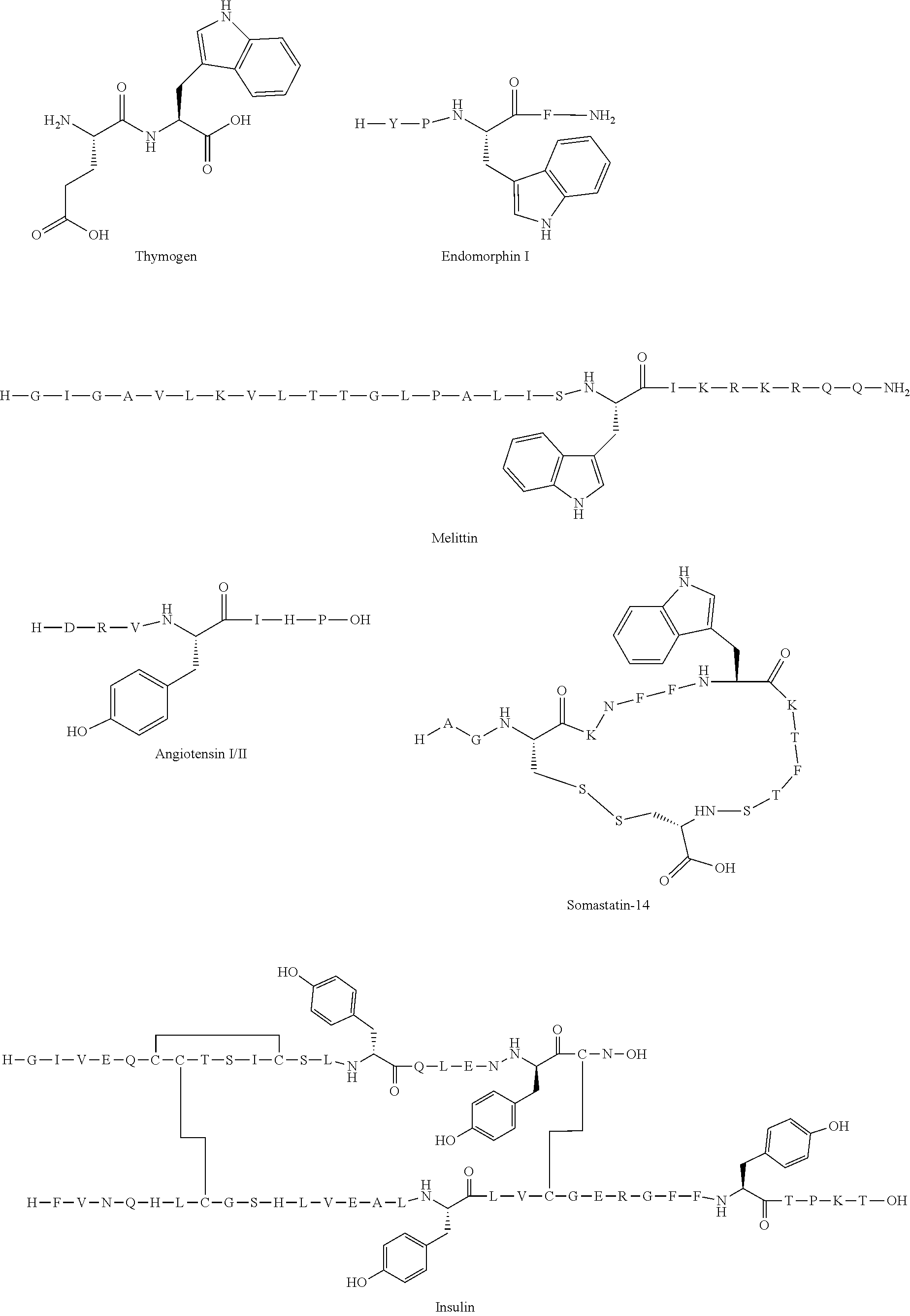

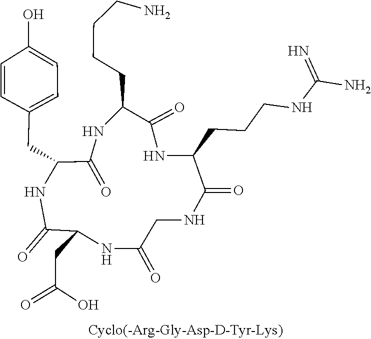 [SMILES: N=C(N)NCCC[C@@H]1NC(=O)[C@H](CCCCN)NC(=O)[C@@H](Cc2ccc(O)cc2)NC(=O)[C@@H](CC(=O)O)NC(=O)CNC1=O]

Activators

The activator generates the ¹⁸F-trifluoromethyl radical from the compound comprising the anion [CF₂¹⁸FSO₂]⁻. Any suitable activator known to the skilled person may be used. Typically, the activator comprises an oxidant, a photosensitizer, a photoredox catalyst or UV light. Examples of activators are discussed in the papers by Li et al. (Li., L et al. Simple and Clean Photoinduced Aromatic Trifluoromethylation Reaction, J. Am. Chem. Soc., 2016, 138, 5809-5812), Wang et al. (Wang., D. Catalyst-free direct C-H trifluoromethylation of arenes in water-acetonitrile, Green Chem., 2016, 18, 5967-5970) and Lefebvre (Lefebvre., Q, Toward Sustainable Trifluoromethylation Reactions: Sodium Triflinate under the Spotlight, Synlett 2017, 28, 19-23).

An oxidant is any substance capable of accepting electrons, thereby oxidising another compound present. Any suitable oxidant may be used. Examples of oxidants are well known to the skilled person. The oxidant may be an organic oxidant or an inorganic oxidant. For instance, the oxidant may be a compound comprising a peroxide group (—O—O—), a compound comprising I(III) or molecular oxygen (O₂).

Examples of compounds comprising I(III) include, but are not limited to diacetoxyiodobenzene derivatives, e.g. phenyliodine bis(trifluoroacetate), and iodine pentoxide.

When the oxidant is a compound comprising a peroxide group, the compound comprising a peroxide group may be a compound of formula R—O—O—R, wherein each R is independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted and unsubstituted heteroaryl and —SO₃⁻. When both R groups are —SO₃⁻ the oxidant may be a persulfate salt, for example sodium persulfate (Na₂S₂O₈).

Preferably, the oxidant is compound of formula R—O—O—R, wherein each R is independently selected from hydrogen and substituted or unsubstituted C₁₋₁₀ alkyl. For instance, one R group may be hydrogen and the other R group may be selected from methyl, ethyl, propyl, n-butyl, s-butyl, i-butyl or t-butyl. Preferably the oxidant is t-butyl hydroperoxide (^{t}BuOOH). Photosensitization is the process by which a photochemical or photophysical alteration occurs in one molecular entity as a result of initial absorption of radiation by another molecular entity called a photosensitizer. The photosensitizer may undergo a chemical change itself. Any suitable photosensitizer may be used. Examples of photosensitizers are well known to the skilled person. Typically when the activator comprises a photosensitizer, contacting the compound comprising an aromatic group with the compound comprising the anion [CF₂¹⁸FSO₂]⁻ in the presence of the photosensitizer is carried out in the presence of light. Typically the light is visible light or UV light.

The photosensitizer may be a compound of formula R[C(O)]ₙR wherein each R is independently selected from H, substituted or unsubstituted Ci-20 alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl and substituted and unsubstituted heteroaryl; wherein the two R groups, together with the —[C(O)]ₙ— group to which they are attached may be bonded together to form a ring; and wherein n is an integer of from 1 to 5.

Typically, the photosensitizer is a compound of formula R[C(O)]ₙR wherein each R is independently substituted or unsubstituted C₁₋₁₀ alkyl or substituted or unsubstituted aryl, and wherein n is 1 or 2. Preferably, the photosensitizer is a compound of formula R[C(O)]ₙR wherein R is substituted or unsubstituted C₁₋₆ alkyl and wherein n is 1 or 2. Preferably, the photosensitizer is acetone, diacetyl or a combination thereof.

A photoredox catalyst is a compound that, when excited by light, can mediate the transfer of electrons. Any suitable photoredox catalyst may be used. Examples of photoredox catalysts are well known to the skilled person. The photoredox catalyst may be an organic photoredox catalyst or an inorganic photoredox catalyst. Typically when the activator comprises a photoredox catalyst, contacting the compound comprising an aromatic group with a compound comprising the anion [CF₂¹⁸FSO₂]⁻ in the presence of the photoredox catalyst is carried out in the presence of light. Typically the light is visible light or UV light.

Examples of photoredox catalysts include, but are not limited to transition metal complexes, such as Ru or Ir complexes, or conjugated organic compounds, such as benzophenone derivatives, anthraquinone derivatives and acridinium derivatives. The photoredox catalyst may be Ru(bipy)₃, Ir[dF(CF₃)ppy]₂(dtbbpy)PF₆, N-Me-mesitylacridinium, anthraquinone-2-carboxylic acid or dimethoxybenzophenone.

The activator may comprise UV light. In one embodiment, the activator is UV light, for instance UV light with a wavelength of less than 280 nm. The activator may comprise UV light and either a photosensitizer or a photoredox catalyst as described herein.

Additive

The step of contacting the compound comprising an aromatic group with a compound comprising the anion [CF₂¹⁸FSO₂]⁻ in the presence of an activator for trifluoromethyl radical formation may be performed in the presence of an additive. The additive may be any compound that influences the rate of the reaction or the product distribution of the reaction.

The additive may be a metal salt, for example a transition metal salt. The additive may be the salt of a first row transition metal, for example a salt of Sc, Ti, V, Cr, Mn, Fe, Co, Ni, Cu or Zn, preferably a salt of Fe. The additive may be a transition metal (III) salt. Preferably, the additive is an Fe(III) salt.

When the additive is a metal salt, the additive will typically comprise an anion. The anion may be selected from any anion described herein. For instance, the one or more anions may be selected from halide, hydroxide, sulfate, and nitrate. Typically the anion is chloride or nitrate. The anion is often nitrate. Thus, the additive may be a metal nitrate salt, typically a transition metal nitrate salt, for instance a first row transition metal nitrate salt, for example iron nitrate. Typically, the additive is Fe(III) nitrate. The metal salt may be a hydrate. For instance, the additive may be Fe(NO₃)₃.9H₂O. The anion is often chloride. The additive may be a metal chloride salt, typically a transition metal chloride salt, for instance a first row transition metal chloride salt, for example iron chloride. Typically the additive is Fe(III) chloride. The metal salt may be a hydrate. For instance, the additive may be FeCl₃.6H₂O.

In one embodiment, the activator is an oxidant as described herein and the additive is a metal salt as described herein. Typically, the activator is a compound comprising a peroxide (—O—O—) group and the additive is a transition metal salt. Preferably, the activator is t-butyl hydroperoxide and the additive is Fe(III) nitrate, preferably Fe(NO₃)₃.9H₂O, or Fe(III) chloride, preferably FeCl₃.6H₂O. The addition of a transition metal salt, such as Fe(NO₃)₃.9H₂O or FeCl₃.6H₂O permits the fast addition of t-butyl hydroperoxide to the reaction mix without oxidation of the [CF₂¹⁸FSO₂]⁻ anion to [CF₂¹⁸FSO₃]⁻.

In the process for producing a compound comprising an ¹⁸F-trifluoromethyl functionalised aromatic group of the invention, the compound comprising the anion [CF₂¹⁸FSO₂]⁻ may be as described herein.

The compound comprising the anion [CF₂¹⁸FSO₂]⁻ may be obtainable by a process as defined herein. The process for producing a compound comprising an ¹⁸F-trifluoromethyl functionalised aromatic group may further comprise a step of obtaining the compound comprising the anion [CF₂¹⁸FSO₂]⁻ by a process as defined herein.

In one embodiment, the process for producing a compound comprising an ¹⁸F-trifluoromethyl functionalised aromatic group comprises contacting a compound comprising an aromatic group with CF₂¹⁸FSO₂NH₄ in the presence of t-butyl hydroperoxide and Fe(NO₃)₃.9H₂O.

Compound Comprising an ¹⁸F-Trifluoromethyl Functionalised Aromatic Group

The present invention also provides a compound comprising an ¹⁸F-trifluoromethyl functionalised aromatic group. The aromatic group may be any aromatic group as described herein.

The compound comprising an ¹⁸F-trifluoromethyl functionalised aromatic group may comprise at least one aromatic group, at least two aromatic groups or at least three aromatic groups as described herein. The compound comprising an ¹⁸F-trifluoromethyl functionalised aromatic group may have a single aromatic group as described herein. The compound comprising an aromatic group may have two aromatic groups as described herein. The compound comprising an aromatic group may have three aromatic groups as described herein.

When the compound comprising a ¹⁸F-trifluoromethyl functionalised aromatic group contains multiple aromatic groups, it is not necessary that all of the aromatic groups are ¹⁸F-trifluoromethyl functionalised. When the compound comprising an aromatic group comprises more than one aromatic group, one of the aromatic groups may be an ¹⁸F-trifluoromethyl functionalised aromatic group. When the compound comprising an aromatic group comprises more than one aromatic group, all of the aromatic groups may be ¹⁸F-trifluoromethyl functionalised aromatic groups. The compound comprising an ¹⁸F-trifluoromethyl functionalised aromatic group may comprise at least one ¹⁸F-trifluoromethyl functionalised aromatic group, at least two ¹⁸F-trifluoromethyl functionalised aromatic groups or at least three ¹⁸F-trifluoromethyl functionalised aromatic groups.

In one embodiment, the compound comprising an ¹⁸F-trifluoromethyl functionalised aromatic group is an amino acid or a compound comprising an amino acid. Thus, the compound may be an ¹⁸F-trifluoromethyl functionalised amino acid, or a compound comprising an ¹⁸F-trifluoromethyl functionalised amino acid. Typically, the amino acid is tyrosine, tryptophan, phenylalanine or histidine. Often the amino acid is tyrosine or tryptophan. In some embodiments the amino acid is tyrosine. In some embodiments the amino acid is tryptophan.

Thus, the compound comprising an ¹⁸F-trifluoromethyl functionalised aromatic group may be ¹⁸F-trifluoromethyl functionalised tyrosine, ¹⁸F-trifluoromethyl functionalised tryptophan, ¹⁸F-trifluoromethyl functionalised phenylalanine or ¹⁸F-trifluoromethyl functionalised histidine. The compound comprising an ¹⁸F-trifluoromethyl functionalised aromatic group may be a compound comprising ¹⁸F-trifluoromethyl functionalised tyrosine, ¹⁸F-trifluoromethyl functionalised tryptophan, ¹⁸F-trifluoromethyl functionalised phenylalanine or ¹⁸F-trifluoromethyl functionalised histidine. Preferably, the compound comprising an ¹⁸F-trifluoromethyl functionalised aromatic group is ¹⁸F-trifluoromethyl functionalised tyrosine or ¹⁸F-trifluoromethyl functionalised tryptophan or a compound comprising ¹⁸F-trifluoromethyl functionalised tyrosine or ¹⁸F-trifluoromethyl functionalised tryptophan.

The compound comprising an ¹⁸F-trifluoromethyl functionalised aromatic group may be a peptide or a protein. Thus, the compound may be an ¹⁸F-trifluoromethyl functionalised peptide or an ¹⁸F-trifluoromethyl functionalised protein.

When the compound comprising an ¹⁸F-trifluoromethyl functionalised aromatic group is a peptide, the peptide may be a dipeptide, a tripeptide, an oligopeptide or a polypeptide. The peptide may be an alkaloid, an anti-microbial agent, a hormone, a growth factor, an immunomodulating agent or an anti-oxidant.

The compound comprising an ¹⁸F-trifluoromethyl functionalised aromatic group may be a peptide or protein comprising ¹⁸F-trifluoromethyl functionalised tyrosine, ¹⁸F-trifluoromethyl functionalised tryptophan, ¹⁸F-trifluoromethyl functionalised phenylalanine or ¹⁸F-trifluoromethyl functionalised histidine. Preferably, the compound comprising an aromatic group is a peptide or protein comprising ¹⁸F-trifluoromethyl functionalised tyrosine or ¹⁸F-trifluoromethyl functionalised tryptophan. The compound comprising an aromatic group may be a peptide or protein comprising ¹⁸F-trifluoromethyl functionalised tyrosine and ¹⁸F-trifluoromethyl functionalised tryptophan.

The compound comprising an ¹⁸F-trifluoromethyl functionalised aromatic group may be a peptide or protein comprising at least one of tyrosine, tryptophan, phenylalanine or histidine, at least two of tyrosine, tryptophan, phenylalanine or histidine or at least three of tyrosine, tryptophan, phenylalanine or histidine. In some instances, the compound comprising a ¹⁸F-trifluoromethyl functionalised aromatic group is a peptide or protein comprising at least one tyrosine residue and at least one tryptophan residue, for example Endomorphin I. In some instances, the compound comprising a ¹⁸F-trifluoromethyl functionalised aromatic group is a peptide or protein comprising at least one tyrosine residue and at least one phenylalanine residue, for example insulin. In some instances, the compound comprising a ¹⁸F-trifluoromethyl functionalised aromatic group is a peptide or protein comprising at least one tyrosine residue and at least one histidine residue, for example Angiotensin I/II or insulin. In some instances, the compound comprising a ¹⁸F-trifluoromethyl functionalised aromatic group is a peptide or protein comprising at least one tryptophan residue and at least one phenylalanine residue, for example Somatostatin-14. In some instances, the compound comprising a ¹⁸F-trifluoromethyl functionalised aromatic group is a peptide or protein comprising at least one tryptophan residue and at least one histidine residue. In some instances, the compound comprising a ¹⁸F-trifluoromethyl functionalised aromatic group is a peptide or protein comprising at least one phenylalanine residue and at least one histidine residue, for example insulin.

The compound comprising a ¹⁸F-trifluoromethyl functionalised aromatic group may be a peptide or protein comprising at least one tyrosine residue, at least two tyrosine residues or at least three tyrosine residues. The compound comprising a ¹⁸F-trifluoromethyl functionalised aromatic group may be a peptide or protein comprising at least one tryptophan residue, at least two tryptophan residues or at least three tryptophan residues. The compound comprising a ¹⁸F-trifluoromethyl functionalised aromatic group may be a peptide or protein comprising at least one phenylalanine residue, at least two phenylalanine residues or at least three phenylalanine residues. The compound comprising a ¹⁸F-trifluoromethyl functionalised aromatic group may be a peptide or protein comprising at least one histidine residue, at least two histidine residues or at least three histidine residues.

The compound comprising an ¹⁸F-trifluoromethyl functionalised aromatic group may be a peptide selected from Thymogen, Endomorphin I, Melittin, Angiotensin I/II, Insulin, Somatostatin-14 and cyclo(-Arg-Gly-Asp-D-Tyr-Lys). For instance, the compound may be Thymogen in which tryptophan is functionalised with a ¹⁸F-trifluoromethyl group, Endomorphin I in which tryptophan is functionalised with a ¹⁸F-trifluoromethyl group, Melittin in which tryptophan is functionalised with a ¹⁸F-trifluoromethyl group, Angiotensin I/II in which tyrosine is functionalised with a ¹⁸F-trifluoromethyl group, insulin in which tyrosine is functionalised with a ¹⁸F-trifluoromethyl group, somatostatin-14 in which tryptophan is functionalised with a ¹⁸F-trifluoromethyl group or cyclo(-Arg-Gly-Asp-D-Tyr-Lys) in which tyrosine is functionalised with a ¹⁸F-trifluoromethyl group.

Thus, the compound comprising ¹⁸F-trifluoromethyl functionalised aromatic group may be selected from:

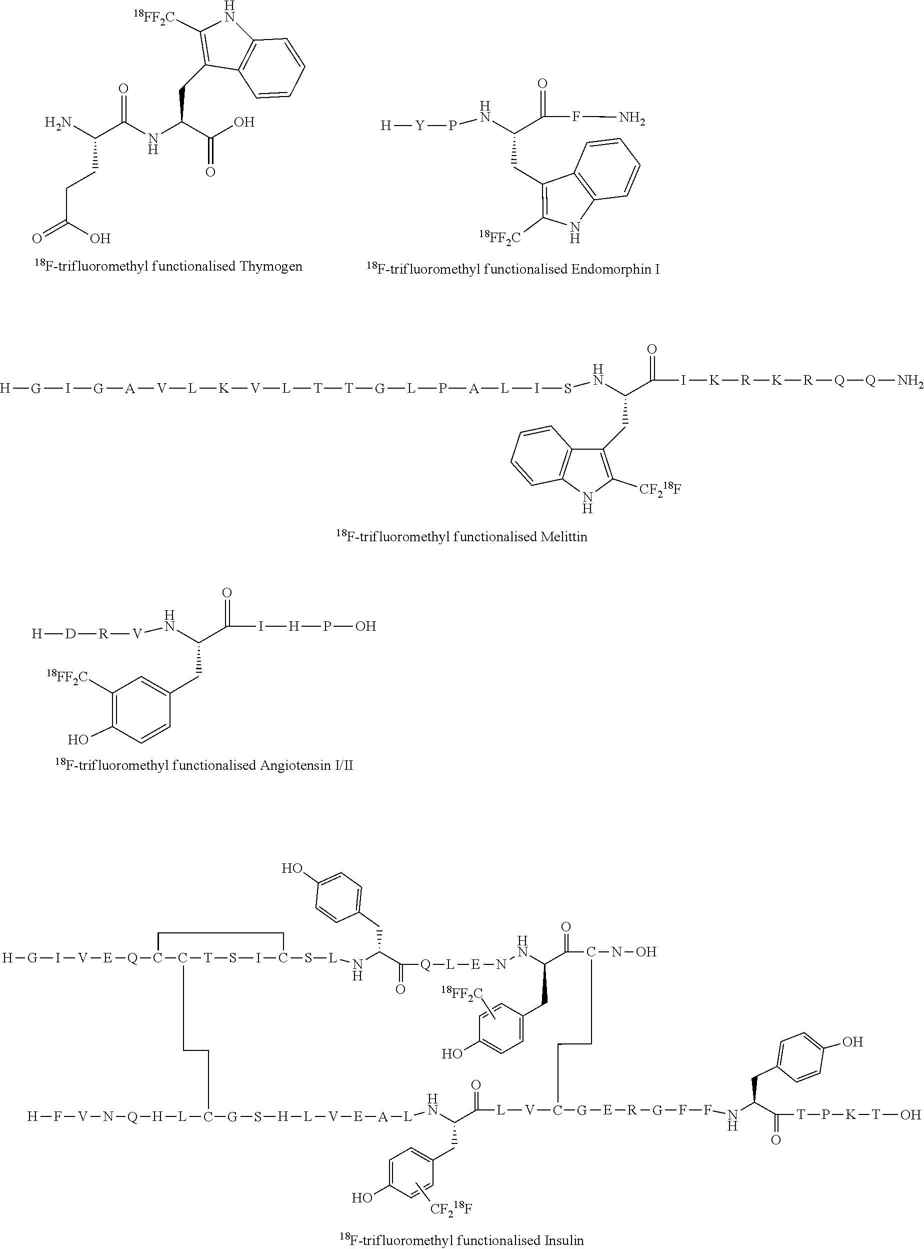

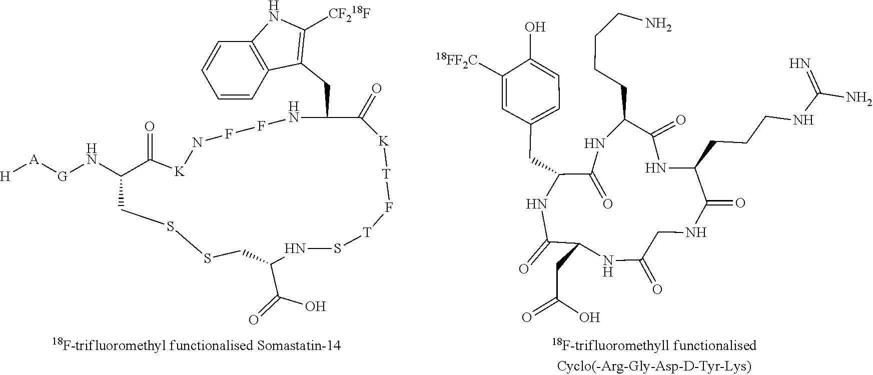

The compound comprising ¹⁸F-trifluoromethyl functionalised aromatic group may be obtainable by a process of the invention as defined herein. The compound comprising ¹⁸F-trifluoromethyl functionalised aromatic group may be obtained by a process of the invention as defined herein.

The invention also provides a pharmaceutical composition comprising (i) a compound comprising an ¹⁸F-trifluoromethyl functionalised aromatic group, or a pharmaceutically acceptable salt thereof, and optionally (ii) one or more pharmaceutically acceptable ingredients.

Suitable pharmaceutically acceptable ingredients are well known to those skilled in the art and include pharmaceutically acceptable carriers (e.g. a saline solution, an isotonic solution), diluents, excipients, adjuvants, fillers, buffers, preservatives, anti-oxidants, lubricants, stabilisers, solubilisers, surfactants (e.g. wetting agents), masking agents, colouring agents, flavouring agents and sweetening agents. Suitable carriers, diluents, excipients, etc. can be found in standard pharmaceutical texts. See, for example, Handbook for Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994.

A pharmaceutical composition may be in the form of (i.e. be formulated as) a liquid, a solution or a suspension (e.g. an aqueous or a non-aqueous solution), an emulsion (e.g. oil-in-water, water-in-oil), an elixir, a syrup, an electuary, a tablet (e.g. coated tablets), granules, a powder, a lozenge, a pastille, a capsule (e.g. hard and soft gelatine capsules), a pill, an ampoule, a bolus, a tincture, a gel, a paste or an oil.

Typically the pharmaceutical composition is suitable for parenteral administration. A pharmaceutical composition suitable for parenteral administration (e.g. by injection) may include an aqueous or non-aqueous, sterile liquid in which the particles employed in the invention are dissolved or suspended. Such liquids may additionally contain other pharmaceutically acceptable ingredients, such as anti-oxidants, buffers, preservatives, stabilisers, bacteriostats, suspending agents, thickening agents, and solutes that render the formulation isotonic with the blood (or other relevant bodily fluid) of the intended recipient. Examples of excipients include water, alcohols, polyols, glycerol, vegetable oils, and the like. Examples of suitable isotonic solutions for use in such formulations include Sodium Chloride Injection, Ringer's Solution or Lactated Ringer's Injection.

The invention also provides a compound comprising an ¹⁸F-trifluoromethyl functionalised aromatic group as described herein for use in a method for treatment of the human or animal body by therapy or for use in a diagnostic method practised on the human or animal body.

The invention also provides a method of treatment comprising administering a therapeutically effective amount of a compound comprising an ¹⁸F-trifluoromethyl functionalised aromatic group as described herein to a subject.

The invention also provides the use of a compound comprising an ¹⁸F-trifluoromethyl functionalised aromatic group as described herein in the manufacture of a medicament.

The invention also provides a method of imaging a subject, comprising administering to the subject a compound comprising ¹⁸F-trifluoromethyl functionalised aromatic group as described herein or a pharmaceutically acceptable salt thereof, and imaging the subject by positron emission tomography (PET). The method may comprise administering to the subject a pharmaceutical composition as described herein.

The invention is further described in the following Examples.

### EXAMPLES

For radiochemistry, the approaches in scheme 1A above are convoluted because they would require a radiosynthetic route towards the necessary [¹⁸F]CF₃-precursor, and one or more reactions post-labeling. Our design plan was to construct [¹⁸F]CF₃SO₂-applying a multi-component approach that would combine ¹⁸F-fluoride, a difluorocarbene source, and SO₂ one-pot. The formation of the ¹⁸F-trifluoromethyl anion from difluorocarbene and ¹⁸F-fluoride is known (Huiban, M et al., Nat. Chem. 2013, 5 (11), 941-944; Zheng, J. et a., Angew. Chem., Int. Ed. 2015, 54 (45), 13236-13240; Zheng, J. et al., Angew. Chem., Int. Ed. 2017, 56 (12), 3196-3200.). A challenge associated with our proposed approach was to validate a protocol that couples the in situ generated [¹⁸F]CF₃⁻ with SO₂, as illustrated in Scheme 1B, preferably but not necessarily using an SO₂ source other than SO₂ itself (so that this gaseous toxic reagent is not needed).

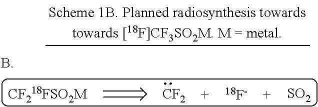

Exploratory studies performed with ¹⁹F-fluoride provided useful information (see below). Both the difluorocarbene and SO₂ sources were found critical to enable the construction of CF₃SO₂⁻. The reaction of (triphenylphosphonio)-difluoroacetate (PDFA) with either 1,4-diazabicyclo[2.2.2]-octane bis(SO₂) adduct (DABSO)³⁰ or N-methyl-morpholine.SO₂ (NMM.SO₂) in the presence of KF/K₂₂₂ in DMF at 120° C. afforded CF₃SO₂K in 63% and 52% yield (¹⁹F NMR), respectively. ClF₂CCO₂Me in combination with PPh₃ was found to be suitable for this process. In contrast to experiments carried out with ¹⁹F-fluoride, DABSO afforded [¹⁸F]CF₃SO₂K in only trace amount (Scheme 2A). The combination of PDFA, NMM.SO₂ and [¹⁸F]KF/K₂₂₂ (typically 20-30 MBq) gave [¹⁸F]CF₃SO₂K in 16% RCC. These results encouraged the development of a protocol to prepare, purify and isolate this novel ¹⁸F-reagent for subsequent use (Scheme 2B). PDFA is thermally unstable and poorly soluble in DMF, so these limitations require that a mixture of this reagent and NMM.SO₂, was added as a suspension in DMF to a vial containing azeotropically dried ¹⁸F-fluoride. Amongst all solvents tested, propylene carbonate (PC) was the most suitable when used with DMF (see See: Yang, Y.; Xu, L.; Yu, S.; Liu, X.; Zhang, Y.; Vicic, D. A. Chem.—A Eur. J. 2016, 22 (3), 858-863). Additional optimization tuning the ratio of reagents and concentration proved beneficial. The optimal process consisted of reacting PDFA (0.16 mmol) and NMM.SO₂ (0.06 mmol) with [¹⁸F]KF/K₂₂₂ (up to 10 GBq) in 350 μL PC/DMF. Initial purification of [¹⁸F]CF₃SO₂K using a weak anion exchange cartridge (WAX) allowed for most of the unreacted ¹⁸F-fluoride and organic byproducts to be removed. Elution with a solution of ˜0.4 M ammonia in EtOH followed by reverse phase HPLC purification afforded [¹⁸F]CF₃SO₂NH₄ in >99% radiochemical purity. Using this protocol, up to 900 MBq of [¹⁸F]CF₃SO₂NH₄ could be isolated from 10 GBq of ¹⁸F-fluoride. The overall nondecay corrected activity yield of isolated [¹⁸F]1 calculated from ¹⁸F-fluoride is 10%±1% (n=7). The identity of [¹⁸F]CF₃SO₂NH₄ was established by HPLC and mass spectrometry ([¹⁹F]CF₃SO₂⁻ (m/z 133.1, calcd 133.0).

Scheme 2. A. One step radiosynthesis of [¹⁸F]CF₃SO₂K from PDFA and
N-methylmorpholine•SO₂ (NMM•SO₂). B. Radiosynthesis, purification
and isolation of [¹⁸F]CF₃SO₂NH₄.

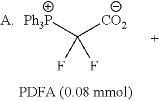

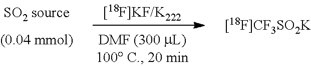

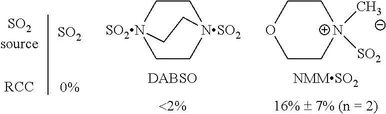

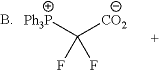

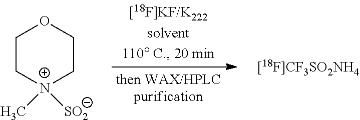

| PDFA | NMM•SO₂ | ¹⁸F-fluoride | solvent | RCY |
|---|---|---|---|---|
| 0.08 mmol | 0.02 mmol | 7.7 GBq | PC^{a,b} | 0% (n = 1) |
| 0.08 mmol | 0.02 mmol | 0.78 GBq | PC + DMF^{c} | 2% (n = 1) |
| 0.16 mmol | 0.06 mmol | 2-10 GBq | PC + DMF^{c} | 10% ± 1% (n = 7)^{d} |

^{a}PC = propylene carbonate. ^{b}300 μL. ^{c}350 μL. ^{d}Radiochemical purity (RCP) > 99%

Studies towards C—H ¹⁸F-trifluoromethylation began using model peptides containing tyrosine and/or tryptophan using t-butyl hydroperoxide (TBHP) as the oxidant. We were faced with immediate challenges. In ¹⁹F-mode, CF₃SO₂Na is added in large excess to enable C—H trifluoromethylation of peptides and proteins (up to ˜200 equiv) (see Imiolek, M.; Karunanithy, G.; Ng, W. L.; Baldwin, A. J.; Gouvemeur, V.; Davis, B. G. J. Am. Chem. Soc. 2018, 140 (5), 1568-1571 and Ji, Y.; Brueckl, T.; Baxter, R. D.; Fujiwara, Y.; Seiple, I. B.; Su, S.; Blackmond, D. G.; Baran, P. S. Proc. Natl. Acad. Sci. 2011, 108 (35), 14411-14415). These conditions are not compatible with ¹⁸F-radiochemisty due to inherent constraints on concentration pertaining to large peptide or proteins, and the ¹⁸F-reagent. An additional complication is competitive oxidation of [¹⁹F]CF₃SO₂NH₄ into [¹⁸F]CF₃SO₃NH₄ in the presence of the initiation reagent. For ¹⁹F-trifluoromethylation, this issue is solved either by using an excess of [¹⁹F]CF₃SO₂Na with respect to TBHP, or via slow addition of TBHP to the reaction mixture (see Ji, Y.; Brueckl, T.; Baxter, R. D.; Fujiwara, Y.; Seiple, I. B.; Su, S.; Blackmond, D. G.; Baran, P. S. Proc. Natl. Acad. Sci. 2011, 108 (35), 14411-14415). These solutions are not suitable for ¹⁸F-labeling because [¹⁸F]CF₃SO₂NH₄ will be the limiting reagent, and operational simplicity is paramount for ¹⁸F-radiochemistry.

The treatment of L-Tyr (0.12 mmol) with [¹⁸F]CF₃SO₂NH₄ (8 MBq) and TBHP (0.12 mmol) in aqueous NH₄HCO₂ (AF) buffer (10% AcOH) did not lead to C—H ¹⁸F-trifluoromethylation at 60° C. after 20 mins. Extensive optimization of the reaction parameters led to [¹⁸F]o-CF₃-L-Tyr in 18% RCC in the presence of both TBHP and Fe(NO₃)₃.9H₂O (Scheme 3A). Higher RCC of 50% was obtained at 60° C. Trifluoromethylation occurred mainly on the o-position, but m-substituted ¹⁸F—CF₃-product was also detected (3% RCC). These two isomers are separable by HPLC. The C—H [¹⁸F]trifluoromethylation of L-Trp was also successful with [¹⁸F]CF₃SO₂NH₄ activated by TBHP and FeC₃. This reaction best performed in DMSO/AF afforded [¹⁸F]CF₃-L-Trp in 27% RCC. The major product is [¹⁸F]2-CF₃-L-Trp (17% RCC) but careful analysis indicated that two regioisomers resulting from competitive ¹⁸F-labeling at the 4- and 7-position were formed (the combined RCC for these two isomers is 10%). The treatment of L-Tyr (0.12 mmol) with [¹⁸F]CF₃SO₂NH₄ (8 MBq) and TBHP (0.12 mmol) in aqueous NH₄HCO₂ (AF) buffer (10% AcOH) did not lead to C—H ¹⁸F-trifluoromethylation at 60° C. after 20 mins. Extensive optimization of the reaction parameters led to [¹⁸F]o-CF₃-L-Tyr in 18% RCC in the presence of both TBHP and Fe(NO₃)₃.9H₂O (Scheme 3A). Higher RCC of 50% was obtained at 60° C. Trifluoromethylation occurred mainly on the o-position, but m-substituted ¹⁸F—CF₃-product was also detected (3% RCC). These two isomers are separable by HPLC. The C—H [¹⁸F]trifluoromethylation of L-Trp was also successful with [¹⁸F]CF₃SO₂NH₄ activated by TBHP and FeCl₃. This reaction best performed in DMSO/AF afforded [¹⁸F]CF₃-L-Trp in 27% RCC. The major product is [¹⁸F]2-CF₃-L-Trp (17% RCC) but careful analysis indicated that two regioisomers resulting from competitive ¹⁸F-labeling at the 4- and 7-position were formed (the combined RCC for these two isomers is 10%).

Next, a series of dipeptides was evaluated with a focus on feasibility and selectivity (Scheme 3B). For reactions leading to more than one ¹⁸F-labeled product, identification was made by comparison of HPLC traces with authentic references prepared independently and fully characterized. Dipeptides Tyr-Trp (Y-W), Trp-Tyr (W-Y) underwent ¹⁸F—CF₃ incorporation exclusively at Trp with higher RCC obtained for the former. For dipeptide Phe-Tyr (F-Y), ¹⁸F-trifluoromethylation occurs at Y affording F-o[^{1B}F]CF₃Y in 37% RCC. Analysis of the crude reaction mixture indicated the formation of minor isomers resulting from [¹′F]CF₃-ation on Y at the meta position and at F. This competitive process was not observed for F-W[¹⁸F]CF₃, a result consistent with the higher reactivity of W versus Y. No competitive ¹⁸F-labeling was detected at His (H) for both H-W[¹⁸F]CF₃ and Y[¹⁸F]CF₃—H. Met (M) oxidation was not observed when ¹⁸F-labeling of M-W (37% RCC), and was largely minimized for M-Y by increasing the Fe:TBHP ratio to 1:1. Oxidative dimerization of cysteine residue however is unavoidable. Next, we studied the ¹⁸F-labeling of biologically relevant peptides of increasing complexity. The dipeptide immunomodulator Thymogen or oglufanide was successfully ¹⁸F-trifluoromethylated at W, and isolated in 37% RCY. Endomorphin 1, a tetrapeptide associated with Alzheimer disease, also underwent W-selective ¹⁸F-labeling in 17% RCY. Similarly, Somatostatin-14, a cyclic tetradecapeptidic hormone with broad inhibitory effect on endocrine secretion, was ¹⁸F-labeled in 20% RCY. The ¹⁸F-trifluoromethylation of the larger 26-residues antimicrobial peptide Melittin was equally successful (18% RCC). Tyrosine-containing peptides were examined next. Angiotensin(1-7), a peptide which shows activity against human lung cancer cells, underwent ¹⁸F-labeling at Tyr in 9%±2% RCC. At this stage, the C—H ¹⁸F-trifluoromethylation of a much larger peptide was considered with recombinant human insulin (MW: about 5800 Da). This experiment carried out with 5.2 μmol of insulin, Fe(NO₃)₃.9H₂O (5.8 equiv) and TBHP (11.5 equiv) in DMSO/25 mM aq. AF (150 μL) led to ¹⁸F—CF₃-insulin as a mixture of four products resulting from [¹⁸F]CF₃ incorporation at all tyrosine residues in 21% overall RCC. The main site of ¹⁸F-trifluoromethylation is chain A Y19, a result consistent with the report of Krsha et al. (see Ichiishi, N.; Caldwell, J. P.; Lin, M.; Zhong, W.; Zhu, X.; Streckfuss, E. C.; Kim, H.-Y. Y.; Parish, C. A.; Krska, S. W. Chem. Sci. 2018, 9 (17), 4168-4175). This is the largest unmodified peptide ¹⁸F-labeled to date.

In conclusion, we have developed the first protocol enabling direct ¹⁸F-labeling of unmodified peptides at the tryptophan and tyrosine residues (with high selectivity for tryptophan) with the CF₃ group via innate C—H functionalization. This convenient method based on the use of readily available ¹⁸F-fluoride is a new tool to accelerate the discovery of ¹⁸F-peptides as imaging agents as well as the development of peptide-based drugs. The strategy required the designed ¹⁸F-isotopologue of the trifluoromethylsulfenate anion [¹⁸F]CF₃SO₂). Considering the number of reactions relying on the Langlois and Baran reagents, we anticipate that the availability of [¹⁸F]CF₃SO₂NH₄ will expand considerably the radiochemical space for PET applications well beyond the peptides defined herein.

General Experimental Information

All NMR spectra were recorded on Bruker AVIII HD 400, AVII 500 and AVIII HD 500 spectrometers. Proton and carbon-13 NMR spectra are reported as chemical shifts (6) in parts per million (ppm) relative to the solvent peak values as given in Gottlieb et al. (Gottlieb, H. E.; Kotlyar, V.; Nudelman, A. J. Org. Chem. 1997, 62 (21), 7512-7515). Fluorine-19 NMR spectra are referenced relative to CFCl₃ in CDCl₃. If trifluoroacetate peak is present, it is used as a reference with values in the relevant solvent as given in Rosenau et al. (Rosenau, C. P.; Jelier, B. J.; Gossert, A. D.; Togni, A.; Rosenau, C. P.; Jelier, B. J.; Gossert, A. D. Angew. Chem., Int. Ed. 2018). Coupling constants (J) are reported in units of hertz (Hz). The following abbreviations are used to describe multiplicities—s (singlet), d (doublet), t (triplet), q (quartet), m (multiplet), br. s (broad singlet). High resolution mass spectra (HRMS, m/z) were recorded on a Bruker MicroTOF spectrometer using positive electrospray ionization (ESI+). Infrared spectra were recorded either as the neat compound or in a solution using a Bruker Tensor 27 FT-IR spectrometer. Absorptions are reported in wavenumbers (cm¹). Optical rotations were measured on a PerkinElmer Polarimeter model 341. Specific rotations are reported in concentrations in g/100 mL. Melting points of solids were measured on a Griffin apparatus and are uncorrected. IUPAC names were obtained using Perkin Elmer Chemdraw Professional Version 16.0.14(77). Solvents were purchased from Sigma-Aldrich, Honeywell and Fisher. Chemicals were purchased from Acros, Alfa Aesar, Bachem, Fluorochem, Sigma-Aldrich and used as received. Peptides were purchased from Bachem and used as received.

The LC-MS/MS analyses were recorded on Xevo G2 Q-TOF coupled to ACQUITY UPLC H-Class LC system (Waters Corporation). ESI ionization source parameters: capillary 3 kV, cone voltage 40 V, source temperature 100° C., desolvation temperature 400° C. and desolvation gas (nitrogen) flow 700 L/h. MS was working at 30,000 (FWHM) resolution. The mass spectrometer operated in data dependent acquisition (DDA) mode with MS survey scan (0.1 s) followed by 0.1 s-0.5 s MS/MS scans on the three most intense ions. To avoid optimization of collision conditions broad MS/MS Collision Energy Ramp 15 V-60 V was used. Leucine enkephalin was used as the lock mass standard. MS/MS data was processed using PEAKS Studio software v8.0 (Bioinformatics Solutions Inc.) with DeNovo analysis with following parameters parent mass error tolerance 10.0 ppm; fragment mass error tolerance 0.02 Da; monoisotopic precursor mass search type. Additional identification in selected cases was accomplished with spectral interpretation software (High Chem Mass Frontier 7.0). Peptide samples (10-50 μg/mL) were prepared in (10-50%) ACN:H₂O mixture with 0.1% FA or TFA. Disulphide containing peptides (insulin, somatostatin) were reduced before LC-MS/MS analysis by mixing with a solution of 1M DTT in 100 mM NH₄OAc buffer (pH 9.0) and 1 h room temperature incubation. The final concentration of DTT was 2 mM and basic pH was confirmed with pH indicator strips. Melittin (0.4 μg) sample was digested at 37° C. with porcine trypsin in 50 mM PBS at pH 8.0 overnight with protein to enzyme ratio 1:25. The reduction/digestion was terminated by acidification with 0.2% FA.

Synthesis Procedure

### Potassium 2-bromo-2,2-difluoroacetate

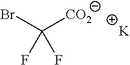

To a solution of potassium hydroxide (5.61 g, 100 mmol) in methanol (90 mL) was added ethyl bromodifluoroacetate (12.8 mL, 100 mmol) before stirring at rt for 12 h. The solvent was evaporated on a rotary evaporator and dried in vacuo to obtain a white solid (19.4 g, 91.1 mmol, 91%). Characterization data is consistent with those reported in the literature.^{3 19}F NMR (377 MHz, Methanol-d₄): δ −58.05 (s). ¹³C NMR (101 MHz, Methanol-d₄) δ 164.98 (t, J=24.9 Hz), 115.22 (t, J=320.4 Hz).

### 2,2-Difluoro-2-(triphenylphosphonio)acetate

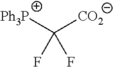

A solution of potassium 2-bromo-2,2-difluoroacetate (2.13 g, 10.0 mmol) and triphenylphosphine (2.62 g, 10.0 mmol) in dry DMF (10 mL) was stirred at rt for 16 h. The mixture was filtered, and the residue was washed with copious amounts of water, acetone and diethyl ether before drying in vacuo to obtain a white solid (2.68 g, 7.53 mmol, 75%). Characterization data is consistent with those reported in the literature.^{3 1}H NMR (400 MHz, Methanol-d₄): δ 7.73-7.81 (m, 2H), 7.82-7.98 (m, 3H); ¹⁹F NMR (377 MHz, Methanol-d₄): δ −96.04 (d, J=96.4 Hz); ³¹P NMR (162 MHz, Methanol-d₄): δ 27.13 (t, J=96.8 Hz).

### 4-Methylmorpholin-4-ium-4-sulfinate

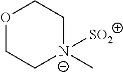

To a dry three neck round-bottom flask containing a magnetic stir bar, with a dry ice/acetone condenser, nitrogen inlet and bubbler attached, was added 4-methylmorpholine (5.94 mL, 50.0 mmol) before cooling with a dry ice/MeCN bath. SO₂ gas was added until approximately 100 mL had condensed. The dry ice/MeCN bath was replaced with a water bath and the excess SO₂ was evaporated while stirring under a nitrogen stream. The resultant solid was dried overnight over P₂O₅ to obtain a pale-yellow solid (7.38 g, 44.7 mmol, 89%). The solid was stored at −20° C. under an inert atmosphere as it is hygroscopic and slowly decomposes in air. ¹H NMR (500 MHz, Methanol-d₄) δ 3.94 (t, J=5.0 Hz, 4H), 3.28 (s, 4H), 2.88 (s, 3H). ¹³C NMR (101 MHz, Methanol-d₄) δ 65.03, 54.47, 44.06, 44.05. ¹⁵N NMR (51 MHz, Methanol-d₄) δ 40.62. Indirect observation from HMBC. IR (solid): 615.0, 623.8, 644.5, 765.2, 859.8, 893.4, 900.5, 936.8, 996.2, 1028.9, 1044, 1067.2, 1088.2, 1112.9, 1156.5, 1181.6, 1198.3, 1280.4, 1313.4, 1370.6, 1460.0, 2866.2, 2966.5, 3026.1. M.P.: 40° C. Analysis calculated for C₅H₁₁NO₃S: C, 36.35; H, 6.71; N, 8.48; 0, 29.05; S, 19.41 found C, 35.32; H, 6.88; N, 8.14; 0, 31.03; S, 18.64.

General Trifluoromethylation Procedure 1

To a mixture of substrate (0.1 mmol), sodium triflinate (15.6 mg, 0.1 mmol), and iron (III) nitrate nonahydrate (40.4 mg, 0.1 mmol) in 1:9 acetic acid/25 mM aqueous ammonium formate (1.0 mL) was added tert-butyl hydroperoxide solution (27.6 μL, 0.2 mmol, 70% in water) before stirring at 40° C. for 20 mins. The solution was taken up in water (10 mL) and added to a Waters Oasis HLB cartridge (activated with 2 mL methanol, 10 mL water) before eluting the crude product with methanol (5.0 mL). The methanol was evaporated, and the desired product was purified via reverse phase preparative HPLC and the collected fractions were lyophilized.

General Trifluoromethylation Procedure 2

To substrate (0.02 mmol) in a 3 mL screw top V-Vials® with open-top cap (Sigma-Aldrich Z115142) was added 100 μL of DMSO, iron (III) nitrate nonahydrate (0.04 mmol, 40 μL of a 1M stock solution in 25 mM aqueous NH₄HCO₂), NaSO₂CF₃ (0.030 mmol, 30 μL of a 1M stock solution in 25 mM aqueous NH₄HCO₂), and 70% TBHP in water (as specified). The vial was placed in a water bath at 40° C. and stirred at 750 rpm for 20-60 mins.

L-Tyr[ortho-CF₃]

### (S)-2-amino-3-(4-hydroxy-3-(trifluoromethyl)phenyl)propanoic acid

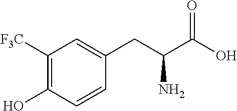 [SMILES: N[C@@H](Cc1ccc(O)c(C(F)(F)F)c1)C(=O)O]

Synthesised following the general trifluoromethylation procedure 1 to obtain a white solid. ¹H NMR (400 MHz, Methanol-d₄) δ 7.46 (d, J=2.2 Hz, 1H), 7.35 (dd, J=8.4, 2.2 Hz, 1H), 6.93 (d, J=8.3 Hz, 1H), 3.76 (dd, J=8.2, 4.6 Hz, 1H), 3.23 (dd, J=14.7, 4.5 Hz, 1H), 3.00 (dd, J=14.7, 8.2 Hz, 1H). Phenolic OH, Carboxylic OH and amine NH₂ were not observed. ¹³C NMR (101 MHz, Methanol-d₄) δ 173.52, 156.56, 135.33, 128.78 (q, J=5.1 Hz), 127.45, 126.52 (q, J=271.1 Hz), 118.30, 118.07 (q, J=30.8 Hz), 57.39, 37.12. ¹⁹F NMR (376 MHz, Methanol-d₄) δ −61.45. HRMS (ESI⁺): for C₁₀H₁₁F₃NO₃ [M+H]⁺ requires m/z=250.0686, found 250.0684.

L-Tyr[meta-CF₃]

### Methyl (tert-butoxycarbonyl)homocysteinate (5)

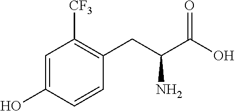 [SMILES: N[C@@H](Cc1ccc(O)cc1C(F)(F)F)C(=O)O]

Synthesised following the general trifluoromethylation procedure 1 to obtain a white solid. ¹H NMR (500 MHz, DMSO-d₆) δ 7.33 (d, J=8.5 Hz, 1H), 7.02 (d, J=2.7 Hz, 1H), 6.94 (dd, J=8.3, 2.6 Hz, 1H). Observed indirectly from COSY as these were obscured by the solvent peak: 3.30, 3.26, 2.71. ¹³C NMR (126 MHz, DMSO-d₆) δ 133.15, 112.24, 118.75, 55.37, 33.72. Observed indirectly from HSQC. ¹⁹F NMR (470 MHz, DMSO-d₆) δ −58.33. HRMS (ESI⁺): for C₁₀H₁₁F₃NO₃ [M+H]⁺ requires m/z=250.0686, found 250.0686.

L-Trp[W-2-CF₃]

### (S)-1-carboxy-2-(2-(trifluoromethyl)-1H-indol-3-yl)ethan-1-aminium trifluoroacetate

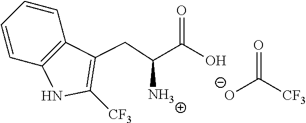 [SMILES: [NH3+][C@@H](Cc1c(C(F)(F)F)[nH]c2ccccc12)C(=O)O ; O=C([O-])C(F)(F)F]

Synthesized following the general trifluoromethylation procedure 2. White solid was obtained after lyophilization. Characterization data is consistent with literature.⁴ Reverse phase HPLC details: Phenomenex Synergi™ 4 μm Hydro RP 80 Å, 250×10 mm. Flow rate=4 mL/min. Column temperature=40° C. Eluent=20% MeCN (0.1% TFA) and 80 H₂O (0.1% TFA) for 3 mins then increase linearly to 27% MeCN at 15 mins and hold at 27% MeCN for another 3 mins. Retention time=9.4 min. ¹H NMR (500 MHz, DMSO-d₆) δ 12.12 (s, 1H), 8.27 (s, 3H), 7.76 (d, J=8.1 Hz, 1H), 7.46 (d, J=8.2 Hz, 1H), 7.31 (t, J=7.6 Hz, 1H), 7.16 (t, J=7.5 Hz, 1H), 3.87 (s, 1H), 3.43-3.27 (m, 2H) partially obscured by water peak. Carboxylic acid OH was not observed. ¹⁹F NMR (471 MHz, DMSO-d₆) δ −57.81, −74.95. MS (ESI⁺): for C₁₂H₁₂F₃N₂O₂ [M+H]⁺ requires 273.1 found 273.1.

Major Trifluoromethylated Side Products of L-Trp Reaction

### (S)-1-carboxy-2-(4-(trifluoromethyl)-1H-indol-3-yl)ethan-1-aminium trifluoroacetate and (S)-1-carboxy-2-(7-(trifluoromethyl)-1H-indol-3-yl)ethan-1-aminium trifluoroacetate (Not Separated)

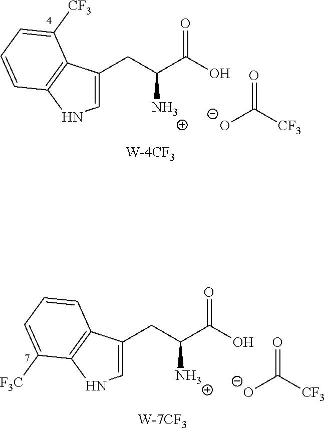 [SMILES: [NH3+][C@@H](Cc1c[nH]c2cccc(C(F)(F)F)c12)C(=O)[O-] ; O=C([O-])C(F)(F)F ; [NH3+][C@@H](Cc1c[nH]c2c(C(F)(F)F)cccc12)C(=O)O ; O=C([O-])C(F)(F)F]

W-4-CF₃ data

Assignment of position on the indole is based on chemical shift of H for the unmodified tryptophan. 4—is more de-shielded than 7—therefore the ¹⁹F of the CF₃ at 4—is expected to be less negative (more positive) than 7-. This assignment is also support by NOESY. Retention time=16.2 mins. ¹H NMR (500 MHz, DMSO-d₆) δ 11.44 (s, 1H), 8.18 (s, 3H), 7.88 (d, J=8.0 Hz, 1H), 7.47 (d, J=7.7 Hz, 1H), 7.36 (d, J=2.6 Hz, 1H), 7.20 (t, J=7.7 Hz, 1H), 4.18 (s, 1H), 3.20-3.32 (2H) partially obscured by water peak. Carboxylic acid OH was not observed. ¹⁹F NMR (471 MHz, DMSO-d₆) δ −57.79, −74.95. Insufficient quantity for ¹³C NMR. HRMS (ESI⁺): for C₁₂H₁₂F₃N₂O₂ [M+H]⁺ requires 273.0845 found 273.0845.

W-7-CF₃ Data

Retention time=16.2 mins. ¹H NMR (500 MHz, DMSO-d₆) δ 11.73 (d, J=2.6 Hz, 1H), 8.18 (s, 3H), 7.74 (d, J=8.1 Hz, 1H), 7.54 (d, J=2.6 Hz, 1H), 7.46 (d, J=7.4 Hz, 1H), 7.27 (t, J=7.8 Hz, 1H), 4.03 (s, 1H), 3.44-3.38 (m, 1H), 3.13 (dd, J=15.7, 9.6 Hz, 1H). Carboxylic acid OH was not observed ¹⁹F NMR (471 MHz, DMSO-d₆) δ −61.22, −74.95. Insufficient quantity for ¹³C NMR. HRMS (ESI⁺): for C₁₂H₁₂F₃N₂O₂ [M+H]+ requires 273.0845 found 273.0845.

TyrTrp[W-2-CF₃]

### (S)-1-(((S)-1-carboxy-2-(2-(trifluoromethyl)-1H-indol-3-yl)ethyl)amino)-3-(4-hydroxyphenyl)-1-oxopropan-2-aminium trifluoroacetate

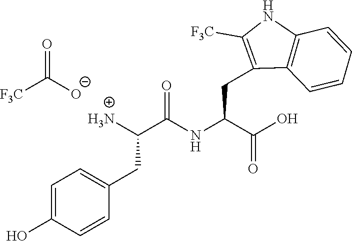 [SMILES: O=C([O-])C(F)(F)F ; [NH3+][C@@H](Cc1ccc(O)cc1)C(=O)N[C@@H](Cc1c(C(F)(F)F)[nH]c2ccccc12)C(=O)O]

Synthesised following the general trifluoromethylation procedure 1 on half the scale to obtain a white solid (9.6 mg, 0.0175 mmol, 35%). Reverse phase HPLC details: Phenomenex Synergi™ 4 μm Hydro RP 80 Å, 250×10 mm. Flow rate=4 mL/min. Column temperature=40° C. Eluent=15% MeCN (0.1% TFA) and 85 H₂O (0.1% TFA) for 3 mins then increase linearly to 49% MeCN at 25 mins. Retention time=16.3 mins. ¹H NMR (500 MHz, DMSO-d₆) δ 12.95 (s, 1H), 12.06 (s, 1H), 9.36 (s, 1H), 9.01 (d, J=8.1 Hz, 1H), 8.04 (s, 3H), 7.78 (d, J=8.1 Hz, 1H), 7.44 (d, J=8.2 Hz, 1H), 7.29 (t, J=7.7 Hz, 1H), 7.15 (t, J=7.5 Hz, 1H), 7.06 (d, J=8.4 Hz, 2H), 6.69 (d, J=8.4 Hz, 2H), 4.57 (q, J=7.9 Hz, 1H), 3.89 (d, J=8.9 Hz, 1H), 3.33 (dd, J=14.6, 8.3 Hz, 1H), 3.23 (dd, J=14.6, 6.7 Hz, 1H), 3.02 (dd, J=14.3, 4.9 Hz, 1H), 2.82 (dd, J=14.3, 8.2 Hz, 1H). ¹³C NMR (126 MHz, DMSO-d₆) δ 172.10, 168.11, 156.62, 135.67, 130.54, 126.73, 124.56, 124.36, 123.22 (q, J=269.3 Hz), 121.66 (q, J=36.1 Hz), 120.11, 120.00, 115.36, 112.38, 112.04 (q, J=2.9 Hz), 53.58, 53.37, 36.01, 26.62. ¹⁹F NMR (376 MHz, DMSO-d₆) δ −57.70, −74.95. HRMS (ESI⁺): for C₂₁H₂₁O₄N₃F₃ [M+H]⁺ requires 436.1479 found 436.1480. [α]_{D}²⁵=+29±5° (c 0.071, H₂O).

Major Trifluoromethylated Side Products of Tyr-Trp Reaction

### (R)-1-(((S)-1-carboxy-2-(7-(trifluoromethyl)-1H-indol-3-yl)ethyl)amino)-3-(4-hydroxyphenyl)-1-oxopropan-2-aminium trifluoroacetate and (R)-1-(((S)-1-carboxy-2-(4-(trifluoromethyl)-1H-indol-3-yl)ethyl)amino)-3-(4-hydroxyphenyl)-1-oxopropan-2-aminium trifluoroacetate (Not Separated)

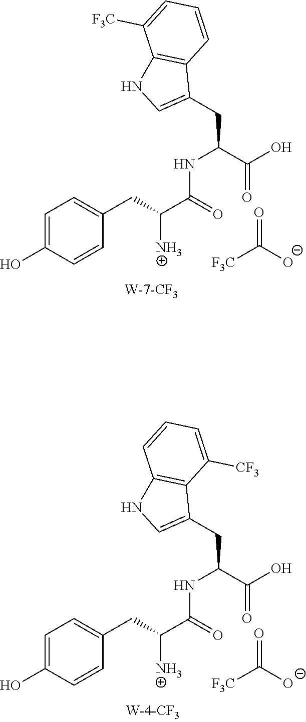 [SMILES: [NH3+][C@H](Cc1ccc(O)cc1)C(=O)N[C@@H](Cc1c[nH]c2c(C(F)(F)F)cccc12)C(=O)O ; O=C([O-])C(F)(F)F ; [NH3+][C@H](Cc1ccc(O)cc1)C(=O)N[C@@H](Cc1c[nH]c2cccc(C(F)(F)F)c12)C(=O)O ; O=C([O-])C(F)(F)F]

Synthesized following the general trifluoromethylation procedure 2: with 0.02 mmol of 70% aqueous TBHP (30 μL of a 2.67M stock solution in 25 mM NH₄HCO₂), reaction time was 50 mins. The crude reaction mixture was diluted with about 2 mL of 5% MCCN (aq) with 0.1% TFA and each time 0.5 mL was injected into the HPLC loop (1 mL) for purification. The yield is not determined as the amount of product is not enough to be measured accurately on an analytical balance (≤0.1 g). Reverse phase HPLC details: Phenomenex Synergi™ 4 μm Hydro RP 80 Å, 250×10 mm. Flow rate=4 mL/min. Column temperature=40° C. Eluent=15% MCCN (0.1% TFA) and 85 H₂O (0.1% TFA) for 3 mins then increase linearly to 49% MeCN at 25 mins. Retention time=19.1 mins (W-4-CF₃ and W-7-CF₃ are not separated).

W-7-CF₃

¹H NMR (500 MHz, DMSO-d₆) δ 12.91 (s, 1H), 11.32 (d, J=2.6 Hz, 1H), 9.33 (s, 1H), 8.81 (s, 1H), 7.95 (s, 3H), 7.87 (d, J=7.9 Hz, 1H), 7.44 (d, J=7.6 Hz, 1H), 7.31 (d, J=2.5 Hz, 1H), 7.18 (t, J=7.7 Hz, 1H), 7.06 (d, J=11.6 Hz, 2H), 6.69 (d, J=8.6 Hz, 2H), 4.66-4.57 (m, 1H), 3.90 (s, 1H), 3.28-3.14 (m, 2H) partially obscured by HDO peak, 3.12-3.02 (m, 1H), 2.81 (dd, J=14.6, 8.4 Hz, 1H). ¹⁹F NMR (471 MHz, DMSO-d₆) δ −61.22, −74.95. Insufficient quantity for ¹³C NMR. HRMS (ESI⁺): for C₂₁H₂₁O₄N₃F₃[M+H]⁺ requires 436.1479 found 436.1489.

W-4-CF₃

¹H NMR (500 MHz, DMSO-d₆) δ 12.91 (s, 1H), 11.59 (d, J=2.7 Hz, 1H), 9.34 (s, 1H), 8.93 (d, J=7.9 Hz, 1H), 7.95 (s, 3H), 7.71 (d, J=8.2 Hz, 1H), 7.47-7.40 (m, 2H), 7.24 (t, J=7.8 Hz, 1H), 7.08 (d, J=8.7 Hz, 2H), 6.69 (d, J=8.5 Hz, 2H), 4.66-4.57 (m, 1H), 3.90 (s, 1H), 3.26-3.14 (m, 1H) partially obscured by HDO peak, 3.12-3.02 (m, 2H), 2.81 (dd, J=14.6, 8.4 Hz, 1H). ¹⁹F NMR (471 MHz, DMSO-d₆) δ −57.63, −74.95. Insufficient quantity for ¹³C NMR. HRMS (ESI⁺): for C₂₁H₂₁O₄N₃F₃[M+H]⁺ requires 436.1479 found 436.1489.

Trp[W-2-CF₃]Tyr

### Tyr(S)-1-(((S)-1-carboxy-2-(4-hydroxyphenyl)ethyl)amino)-1-oxo-3-(2-(trifluoromethyl)-1H-indol-3-yl)propan-2-aminium trifluoroacetate

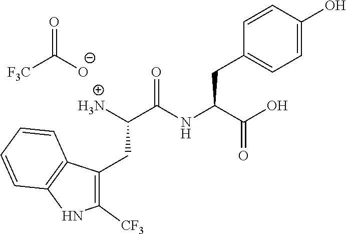 [SMILES: [NH3+][C@@H](Cc1c(C(F)(F)F)[nH]c2ccccc12)C(=O)N[C@@H](Cc1ccc(O)cc1)C(=O)O ; O=C([O-])C(F)(F)F]

Synthesised following the general trifluoromethylation procedure 1 to obtain a white solid (11.0 mg, 0.0200 mmol, 20%). Reverse phase HPLC details: C18(2) Luna, 250×10 mm 5p 100 Å. Flow rate=4 mL/min. Column temperature=25° C. Eluent=19% MeCN (0.1% TFA) in 81% water (0.1% TFA). Retention time=17 mins. ¹H NMR (500 MHz, DMSO-d₆) δ 12.73 (s, 1H), 12.09 (s, 1H), 9.21 (s, 1H), 8.62 (d, J=7.8 Hz, 1H), 8.27 (s, 3H), 7.79 (d, J=8.1 Hz, 1H), 7.44 (d, J=8.2 Hz, 1H), 7.29 (t, J=7.6 Hz, 1H), 7.15 (t, J=7.5 Hz, 1H), 6.98-6.91 (m, 2H), 6.66-6.59 (m, 2H), 4.37 (dt, J=8.0, 6.3 Hz, 1H), 4.14-4.08 (m, 1H), 3.38-3.29 (m, 1H), 3.17 (dd, J=14.9, 6.8 Hz, 1H), 2.88 (dd, J=13.9, 5.9 Hz, 1H), 2.82 (dd, J=13.9, 6.6 Hz, 1H). ¹C NMR (126 MHz, DMSO-d₆) δ 171.58, 167.64, 157.68 (q, J=30.6 Hz), 156.06, 135.77, 130.23, 126.78, 126.61, 124.38, 122.63 (q, J=36.0 Hz), 121.89 (q, J=269.2 Hz), 120.12, 120.00, 117.39 (q, J=301.3 Hz), 114.98, 112.38, 109.21 (q, J=2.6 Hz), 54.01, 52.41, 36.31, 26.28. ¹⁹F NMR (471 MHz, DMSO-d₆) δ −57.59, −74.95. HRMS (ESI⁺): for C₂₁H₂₁O₄N₃F₃ [M+H]⁺ requires 436.1479 found 436.1476. [α]_{D}²⁵=+53±1° (c 0.065, H₂O).

Major Trifluoromethylated Side Products of Trp-Tyr Reaction

### (S)-1-(((S)-1-carboxy-2-(4-hydroxyphenyl)ethyl)amino)-1-oxo-3-(7-(trifluoromethyl)-1H-indol-3-yl)propan-2-aminium trifluoroacetate

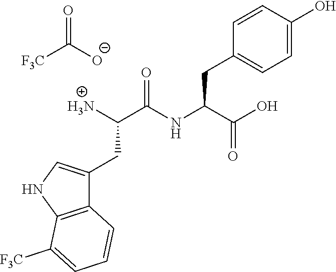 [SMILES: [NH3+][C@@H](Cc1c[nH]c2c(C(F)(F)F)cccc12)C(=O)N[C@@H](Cc1ccc(O)cc1)C(=O)O ; O=C([O-])C(F)(F)F]

Synthesized following the general trifluoromethylation procedure 2: with 0.02 mmol of 70% aqueous TBHP (30 μL of a 2.67M stock solution in 25 mM NH₄HCO₂), reaction time was 30 mins. The crude reaction mixture was diluted with about 2 mL of 5% MeCN (aq) with 0.1% TFA and each time 0.5 mL was injected into the HPLC loop (1 mL) for purification. The yield is not determined as the amount of product is not enough to be measured accurately on an analytical balance (≤0.1 g). Reverse phase HPLC details: Phenomenex Synergi™ 4 μm Hydro RP 80 Å, 250×10 mm. Column temperature=25° C. Flow rate=4 mL/min. Column temperature=40° C. Eluent=17% MeCN (0.1% TFA) and 83% H₂O (0.1% TFA) for 3 mins then increase linearly to 32% MeCN at 25 mins then another linear increase to 36% MeCN at 27 mins. Retention time=21.6. mins. ¹H NMR (500 MHz, DMSO-d₆) δ 11.69 (s, 1H), 9.23 (s, 1H), 8.73 (s, 1H), 7.71 (d, J=8.1 Hz, 1H), 7.54 (d, J=2.7 Hz, 1H), 7.43 (d, J=7.5 Hz, 1H), 7.25 (t, J=7.8 Hz, 1H), 7.05-7.00 (m, 2H), 6.69-6.63 (m, 2H), 4.47-4.39 (m, 1H), 4.22 (s, 1H), 3.33-3.07 (m, 2H), 2.96 (dd, J=14.1, 5.3 Hz, 1H), 2.83 (dd, J=14.0, 8.3 Hz, 1H). Protons of NH₃ (N-terminal) and the alpha proton adjacent to the NH₃ group are not observed probably due to exchange. benzylic protons of phenol are probably obscured by HDO peak. ¹⁹F NMR (471 MHz, DMSO-d₆) δ −57.30, −74.95. Insufficient quantity for ¹³C NMR. HRMS (ESI⁺): for C₂₁H₂₁O₄N₃F₃[M+H]⁺ requires 436.1479 found 436.1507.

### (S)-1-(((S)-1-carboxy-2-(4-hydroxyphenyl)ethyl)amino)-1-oxo-3-(4-(trifluoromethyl)-1H-indol-3-yl)propan-2-aminium trifluoroacetate

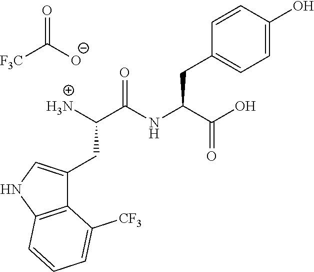 [SMILES: [NH3+][C@@H](Cc1c[nH]c2cccc(C(F)(F)F)c12)C(=O)N[C@@H](Cc1ccc(O)cc1)C(=O)O ; O=C([O-])C(F)(F)F]

Synthesized following the general trifluoromethylation procedure 2: with 0.02 mmol of 70% aqueous TBHP (30 μL of a 2.67M stock solution in 25 mM NH₄HCO₂), reaction time was 30 mins. The crude reaction mixture was diluted with about 2 mL of 5% MCCN (aq) with 0.1% TFA and each time 0.5 mL was injected into the HPLC loop (1 mL) for purification. The yield is not determined as the amount of product is not enough to be measured accurately on an analytical balance (≤0.1 g). Reverse phase HPLC details: Phenomenex Synergi™ 4 μm Hydro RP 80 Å, 250×10 mm. Flow rate=4 mL/min. Column temperature=40° C. Eluent=17% MCCN (0.1% TFA) and 83% H₂O (0.1% TFA) for 3 mins then increase linearly to 32% MCCN at 25 mins then another linear increase to 36% MeCN at 27 mins. Retention time=24.0. mins. ¹H NMR (500 MHz, DMSO-d₆) δ 12.98 (s, 1H), 11.41 (s, 1H), 9.25 (s, 1H), 8.92 (s, 1H), 8.03 (d, J=8.1 Hz, 1H), 7.98 (s, 3H), 7.46 (d, J=7.6 Hz, 1H), 7.35 (d, J=2.5 Hz, 1H), 7.19 (t, J=7.7 Hz, 1H), 7.05 (d, J=8.0 Hz, 2H), 6.68 (d, J=8.4 Hz, 2H), 4.47 (q, J=7.2 Hz, 1H), 4.03 (s, 1H), 3.34-3.21 (m, 1H), 3.08 (dd, J=14.9, 9.2 Hz, 1H), 3.05-2.96 (m, 1H) partially obscured by HDO peak, 2.87 (dd, J=14.0, 8.3 Hz, 1H). ¹⁹F NMR (471 MHz, DMSO-d₆) δ −61.18, −74.95. Insufficient quantity for ¹³C NMR. HRMS (ESI⁺): for C₂₁H₂₁O₄N₃F₃[M+H]⁺ requires 436.1479 found 436.1465.

PheTyr[ortho-CF₃]

### (S)-1-(((S)-1-carboxy-2-(4-hydroxy-3-(trifluoromethyl)phenyl)ethyl)amino)-1-oxo-3-phenylpropan-2-aminium trifluoroacetate

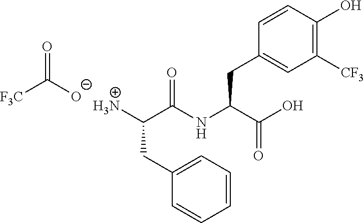 [SMILES: O=C([O-])C(F)(F)F ; [NH3+][C@@H](Cc1ccccc1)C(=O)N[C@@H](Cc1ccc(O)c(C(F)(F)F)c1)C(=O)O]

Synthesised following the general trifluoromethylation procedure 1 to obtain a white solid (34.7 mg, 0.0680 mmol, 68%). Reverse phase HPLC details: C18(2) Luna, 250×10 mm 5μ 100 Å. Flow rate=4 mL/min. Column temperature=25° C. Eluent=28% MeCN (0.1% TFA) and 72% H₂O (0.1% TFA). Retention time=10 mins. ¹H NMR (500 MHz, DMSO-d₆) δ 10.50 (s, 1H), 8.85 (d, J=7.9 Hz, 1H), 8.08 (s, 4H), 7.38 (d, J=2.1 Hz, 1H), 7.35-7.24 (m, 7H), 6.95 (d, J=8.4 Hz, 1H), 4.47 (td, J=7.9, 5.2 Hz, 1H), 4.02 (dd, J=8.5, 4.8 Hz, 1H), 2.91 (ddd, J=14.4, 8.3, 6.2 Hz, 2H). Carboxylic acid-OH is not observed. ¹³C NMR (126 MHz, DMSO-d₆) δ 172.10, 168.20, 157.96, 157.71, 154.58, 134.71, 134.47, 129.56, 128.55, 127.20, 127.16, 127.12, 124.06 (q, J=272.7 Hz), 116.91, 115.09 (q, J=29 Hz), 53.84, 53.16, 36.98, 35.60. ¹F NMR (470 MHz, DMSO-d₆) δ −62.25, −74.95. HRMS (ESI⁺): for C₁₉H₂₀O₄N₂F₃ [M+H]⁺ requires 397.1370 found 397.1371. [α]_{D}²⁵=+28±2° (c 0.044, H₂O).

Major Trifluoromethylated Side Products of Phe-Tyr Reaction

Synthesized following the general trifluoromethylation procedure 2: with 0.08 mmol of 70% aqueous TBHP (30 μL of a 2.67M stock solution in 25 mM NH₄HCO₂), reaction time was 40 mins. The crude reaction mixture was diluted with about 2 mL of 5% MeCN (aq) with 0.1% TFA and each time 0.5 mL was injected into the HPLC loop (1 mL) for purification. The yield is not determined as the amount of product is not enough to be measured accurately on an analytical balance (≤0.1 g). Reverse phase HPLC details: Phenomenex Synergi™ 4 μm Hydro RP 80 Å, 250×10 mm. Column temperature=40° C. Flow rate=4 mL/min. Column temperature=40° C. Eluent=20% MCCN (0.1% TFA) and 80% H₂O (0.1% TFA) for 3 mins then increase linearly to 30% MeCN at 25 mins.

Phe[meta-CF₃]-Tyr and Phe-Tyr[meta-CF₃] (Not separated)

Retention time=18-19.1 mins.

Phe-Tyr[meta-CF₃]

¹H NMR (500 MHz, DMSO-d₆) δ 10.03 (s, 1H), 8.98 (d, J=8.3 Hz, 1H), 8.06 (s, 3H), 7.41-7.21 (m, 6H), 7.05 (d, J=2.7 Hz, 1H), 6.97 (dd, J=8.4, 2.7 Hz, 1H), 4.63-4.38 (m, 1H), 4.00 (dd, J=8.2, 4.9 Hz, 1H), 3.23-3.09 (m, 2H), 3.03-2.85 (m, 2H). Carboxylic acid —OH is not observed. ¹⁹F NMR (470 MHz, DMSO-d₆) δ −59.96, −74.95. Insufficient quantity for ¹³C NMR. MS (ESI⁺): for C₁₉H₂₀O₄N₂F₃ [M+H]⁺ requires 397.14 found 397.15.

Phe[meta-CF₃]-Tyr

¹H NMR (500 MHz, DMSO-d₆) δ 9.27 (s, 1H), 8.78 (d, J=7.9 Hz, 1H), 8.06 (s, 3H), 7.68 (s, 1H), 7.66-7.62 (m, 1H), 7.61-7.51 (m, 2H), 7.04 (d, J=10.0 Hz, 2H), 6.68 (d, J=8.4 Hz, 2H), 4.56-4.37 (m, 1H), 4.09 (dd, J=8.8, 4.5 Hz, 1H), 3.22-3.11 (m, 2H), 3.05-2.98 (m, 1H), 2.85 (dd, J=14.0, 8.2 Hz, 1H). Carboxylic acid —OH is not observed. ¹⁹F NMR (470 MHz, DMSO-d₆) δ −62.48, −74.95. Insufficient quantity for ¹³C NMR. MS (ESI⁺): for C₁₉H₂₀O₄N₂F₃ [M+H]⁺ requires 397.14 found 397.15.

NMR Analysis

The chemical shifts of the OH protons of tyrosine provide strong evidence that the phenol group of one of the tyrosine residues is not trifluoromethylated. The main motivation to postulate that this is the Phe-meta-CF₃ is that one of the aromatic protons in the 7.5-7.7 ppm region is a broad singlet. This pattern is inconsistent with Phe-ortho-CF₃ and Phe-para-CF₃. The ¹³C chemical shifts observed indirectly via HSQC are also consistent with a meta-CF₃ on the phenyl group of the Phe residue (refer to the ¹³C NMR of 3-Trifluoromethyltoluene in Knauber, T. et al., L. J. Chem.—A Eur. J. 2011, 17 (9), 2689-2697).

Phe[para-CF₃]-Tyr (Not separated one unidentified impurity with semi-prep column) Retention time=19.4-20.1 mins. ¹⁹F NMR (470 MHz, DMSO-d₆) δ −62.41, −74.95. HRMS (ESI⁺): for C₁₉H₂₀O₄N₂F₃ [M+H]⁺ requires 397.1370 found 397.1388.

NMR Analysis

The chemical shifts of the OH protons of tyrosine residue provide strong evidence that the phenol group of one of the tyrosine residues is not trifluoromethylated. The integration of the amide's NH relative phenol's OH suggested the presence of molecule(s) that does not contain a phenol's OH. Phe[para-CF₃]-Tyr is identified by two distinct doublets in the ¹H spectrum and their coupling from COSY90. While it is tempting to assign the minor peaks which overlap the two doublets of Phe aromatic protons of Phe[para-CF₃]-Tyr to Phe[ortho-CF₃]-Tyr, the coupling constant is too small in magnitude to be due to at ³J_{HH}. The 19F chemical shift of Phe[ortho-CF₃] is expected to be smaller in magnitude than both meta- and para- as reported in Knauber, T. et al., L. J. Chem.—A Eur. J. 2011, 17 (9), 2689-2697.

PheTrp[W-2-CF₃]

### (S)-1-(((S)-1-carboxy-2-(2-(trifluoromethyl)-1H-indol-3-yl)ethyl)amino)-1-oxo-3-phenylpropan-2-aminium 2,2,2-trifluoroacetate

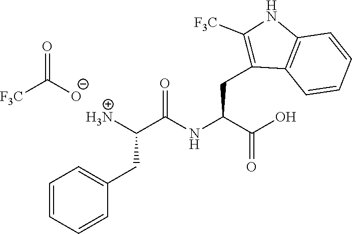 [SMILES: O=C([O-])C(F)(F)F ; [NH3+][C@@H](Cc1ccccc1)C(=O)N[C@@H](Cc1c(C(F)(F)F)[nH]c2ccccc12)C(=O)O]

Synthesised following the general trifluoromethylation procedure 1 to obtain a white solid (9.5 mg, 0.0178 mmol, 18%). Reverse phase HPLC details: Phenomenex Synergi™ 4 μm Hydro RP 80 Å, 250×10 mm. Flow rate=4 mL/min. Column temperature=40° C. Eluent=15% MCCN (0.1% TFA) and 85% H₂O (0.1% TFA) for 3 mins then increase linearly to 49% MCCN at 25 mins. Retention time=18.8 mins. ¹H NMR (500 MHz, DMSO-d₆) δ 12.92 (s, 1H), 12.07 (s, 1H), 9.04 (d, J=8.2 Hz, 1H), 8.17-8.13 (m, 3H), 7.79 (d, J=8.1 Hz, 1H), 7.45 (d, J=8.3 Hz, 1H), 7.37-7.23 (m, 6H), 7.15 (ddd, J=8.0, 6.8, 1.0 Hz, 1H), 4.57 (td, J=8.2, 6.5 Hz, 1H), 4.00 (d, J=5.6 Hz, 1H), 3.36-3.28 (m, 1H), 3.27-3.19 (m, 1H), 3.13 (dd, J=14.2, 5.2 Hz, 1H), 2.95 (dd, J=14.1, 8.1 Hz, 1H). ¹³C NMR (126 MHz, DMSO-d₆) δ 172.04, 167.98, 135.68, 134.72, 129.54, 128.56, 127.22, 126.73, 124.36, 122.16 (q, J=269.1 Hz), 121.68 (q, J=36.1 Hz), 120.11, 120.00, 112.40, 112.00 (q, J=2.8 Hz), 53.38, 53.32, 36.81, 26.67. ¹⁹F NMR (471 MHz, DMSO-d₆) δ −57.48, −74.95. HRMS (ESI⁺): for C₂₁H₂₁F₃N₃O₃ [M+H]⁺ requires 420.1530 found 420.1527. [α]_{D}²⁵=−71±3° (c 0.051, H₂O).

Major Trifluoromethylated Side Products of Phe-Trp Reaction

### (S)-1-(((S)-1-carboxy-2-(7-(trifluoromethyl)-1H-indol-3-yl)ethyl)amino)-1-oxo-3-phenylpropan-2-aminium trifluoroacetate

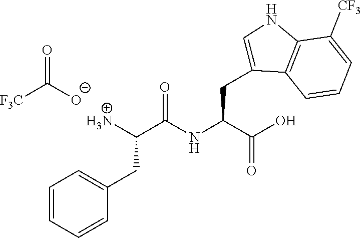 [SMILES: O=C([O-])C(F)(F)F ; [NH3+][C@@H](Cc1ccccc1)C(=O)N[C@@H](Cc1c[nH]c2c(C(F)(F)F)cccc12)C(=O)O]

Synthesized following the general trifluoromethylation procedure 2: with 0.02 mmol of 70% aqueous TBHP (30 μL of a 2.67M stock solution in 25 mM NH₄HCO₂), reaction time was 50 mins. The crude reaction mixture was diluted with about 2 mL of 5% MCCN (aq) with 0.1% TFA and each time 0.5 mL was injected into the HPLC loop (1 mL) for purification. The collected fractions were lyophilized to give a white solid. The yield is not determined as the amount of product is not enough to be measured accurately on an analytical balance (≤0.1 g). Reverse phase HPLC details: Phenomenex Synergi™ 4 μm Hydro RP 80 Å, 250×10 mm. Flow rate=4 mL/min. Column temperature=40° C. Eluent=15% MeCN (0.1% TFA) and 85% H₂O (0.1% TFA) for 3 mins then increase linearly to 49% MCCN at 25 mins. Retention time=21.1 mins. ¹H NMR (500 MHz, DMSO-d₆) δ 11.31 (s, 1H), 7.85 (d, J=7.9 Hz, 1H), 7.44 (d, J=7.4 Hz, 1H), 7.33-7.22 (m, 6H), 7.17 (t, J=7.6 Hz, 1H), 4.59 (t, J=6.8 Hz, 11H), 3.26-3.10 (m, 2H) Partially obscured by solvent peak. Protons of NH₃ (N-terminal) and the alpha proton adjacent to the NH₃ group are not observed probably due to exchange. benzylic protons of phenol are probably obscured by HDO peak. ¹⁹F NMR (470 MHz, DMSO-d₆) δ −61.23, −74.95. Insufficient quantity for ¹³C NMR. MS (ESI⁺): for C₂₁H₂₁F₃N₃O₃ [M+H]⁺ requires 420.15 found 420.15.

### (S)-1-(((S)-1-carboxy-2-(4-(trifluoromethyl)-1H-indol-3-yl)ethyl)amino)-1-oxo-3-phenylpropan-2-aminium trifluoroacetate

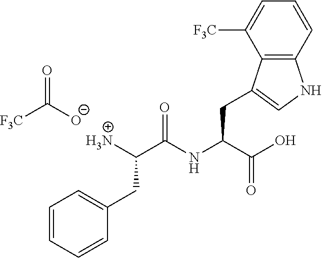 [SMILES: O=C([O-])C(F)(F)F ; [NH3+][C@@H](Cc1ccccc1)C(=O)N[C@@H](Cc1c[nH]c2cccc(C(F)(F)F)c12)C(=O)O]

Synthesized following the general trifluoromethylation procedure 2: with 0.02 mmol of 70% aqueous TBHP (30 μL of a 2.67M stock solution in 25 mM NH₄HCO₂), reaction time was 50 mins. The crude reaction mixture was diluted with about 2 mL of 5% MCCN (aq) with 0.1% TFA and each time 0.5 mL was injected into the HPLC loop (1 mL) for purification. The collected fractions were lyophilized to give a white solid. The yield is not determined as the amount of product is not enough to be measured accurately on an analytical balance (≤50.1 g).

Reverse phase HPLC details: Phenomenex Synergi™ 4 μm Hydro RP 80 Å, 250×10 mm. Flow rate=4 mL/min. Column temperature=40° C. Eluent=15% MeCN (0.1% TFA) and 85% H₂O (0.1% TFA) for 3 mins then increase linearly to 49% MCCN at 25 mins. Retention time=21.5 mins. ¹H NMR (500 MHz, DMSO-d₆) δ 11.58 (s, 1H), 7.71 (d, J=8.1 Hz, 1H), 7.43 (d, J=7.5 Hz, 1H), 7.41 (s, 1H), 7.36-7.27 (m, 6H), 7.24 (t, J=7.8 Hz, 1H), 4.63 (q, J=7.5 Hz, 1H), 3.21-2.97 (m, 2H) Partially obscured by solvent peak. Protons of NH₃ (N-terminal) and the alpha proton adjacent to the NH₃ group are not observed probably due to exchange. benzylic protons of phenol are probably obscured by HDO peak. ¹⁹F NMR (470 MHz, DMSO-d₆) δ −57.62, −74.95. Insufficient quantity for ¹³C NMR. HRMS (ESI⁺): for C₂₁H₂₁F₃N₃O₃ [M+H]⁺ requires 420.15 found 420.16.

MetTrp[W-2-CF₃]

### (S)-1-(((S)-1-carboxy-2-(2-(trifluoromethyl)-1H-indol-3-yl)ethyl)amino)-4-(methylthio)-1-oxobutan-2-aminium trifluoroacetate

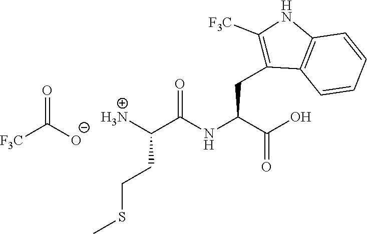 [SMILES: O=C([O-])C(F)(F)F ; CSCC[C@H]([NH3+])C(=O)N[C@@H](Cc1c(C(F)(F)F)[nH]c2ccccc12)C(=O)O]

Synthesised following the general trifluoromethylation procedure 1 to obtain a white solid after lyophilization (5.1 mg, 0.00986 mmol, 10%). Reverse phase HPLC details: Phenomenex Synergi™ 4 μm Hydro RP 80 Å, 250×10 mm. Flow rate=4 mL/min. Column temperature=40° C. Eluent=5% MeCN (0.1% TFA) and 95% H₂O (0.1% TFA) for 3 mins then increase linearly to 40% MeCN at 25 mins. Retention time=23.0 mins. ¹H NMR (500 MHz, DMSO-d₆) δ 12.72 (s, 1H), 12.07 (s, 1H), 8.96 (d, J=8.0 Hz, 1H), 8.20 (s, 3H), 7.76 (d, J=8.1 Hz, 1H), 7.44 (dd, J=8.3, 1.0 Hz, 1H), 7.29 (ddd, J=8.2, 6.9, 1.1 Hz, 1H), 7.14 (ddd, J=8.1, 6.9, 1.0 Hz, 1H), 4.54 (q, J=7.6 Hz, 1H), 3.80 (d, J=6.3 Hz, 1H), 3.36-3.30 (m, 1H), 3.26-3.18 (m, 1H), 2.53-2.42 (m, 2H), 2.05 (s, 3H), 2.04-1.96 (m, 1H). ¹³C NMR (126 MHz, DMSO-d₆) δ 172.19, 168.15, 135.65, 126.75, 124.34, 122.17 (q, J=269.1 Hz), 121.63 (q, J=36.2 Hz), 120.11, 119.98, 112.38, 112.20 (q, J=2.9 Hz), 53.51, 51.51, 30.89, 27.88, 26.07, 14.43. ¹⁹F NMR (470 MHz, DMSO-d₆) δ −57.71, −74.95. HRMS (ESI⁺): for C₁₇H₂₁F₃N₃O₃³²S [M+H]⁺ requires 404.1250 found 404.1253. [α]_{D}²⁵=+18±1° (c 0.076, H₂O).

Major Trifluoromethylated Side Products of Met-Trp Reaction

### (S)-1-(((S)-1-carboxy-2-(7-(trifluoromethyl)-1H-indol-3-yl)ethyl)amino)-4-(methylthio)-1-oxobutan-2-aminium and (S)-1-(((S)-1-carboxy-2-(4-(trifluoromethyl)-1H-indol-3-yl)ethyl)amino)-4-(methylthio)-1-oxobutan-2-aminium (Not Separated)

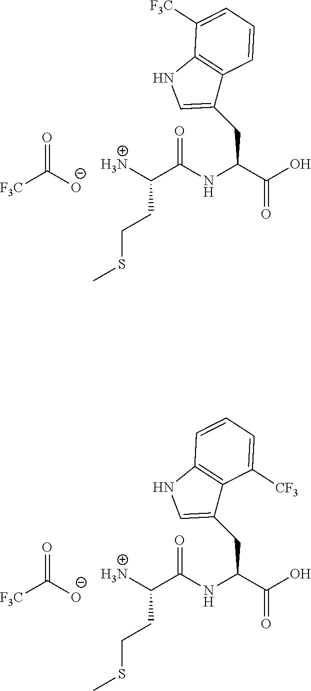 [SMILES: O=C([O-])C(F)(F)F ; CSCC[C@H]([NH3+])C(=O)N[C@@H](Cc1c[nH]c2c(C(F)(F)F)cccc12)C(=O)O ; O=C([O-])C(F)(F)F ; CSCC[C@H]([NH3+])C(=O)N[C@@H](Cc1c[nH]c2cccc(C(F)(F)F)c12)C(=O)O]

Synthesized following the general trifluoromethylation procedure 2 but with 0.01 mmol of substrate (all reagents were halved): with 0.02 mmol of 70% aqueous TBHP (10.7 μL of a 2.67M stock solution in 25 mM NH₄HCO₂), reaction time was 25 mins. The crude reaction mixture was diluted with about 0.9 mL of 5% MeCN (aq) with 0.1% TFA and injected into the HPLC loop (1 mL) for purification. Reverse phase HPLC details: Phenomenex Synergi™ 4 μm Hydro RP 80 Å, 250×10 mm. Flow rate=4 mL/min. Column temperature=40° C. Eluent=5% MeCN (0.1% TFA) and 95% H₂O (0.1% TFA) for 3 mins then increase linearly to 40% MeCN at 25 mins. Retention time=25.6 mins. (W-4-CF₃ and W-7-CF₃ are not separated)

W-7-CF₃

¹H NMR (500 MHz, DMSO-d₆) δ 12.97 (s, 1H), 11.32 (d, J=2.6 Hz, 1H), 8.75 (d, J=7.5 Hz, 1H), 8.10 (s, 3H), 7.87 (d, J=8.0 Hz, 1H), 7.45 (d, J=7.0 Hz, 1H), 7.32 (d, J=2.5 Hz, 1H), 7.18 (t, J=7.7 Hz, 1H), 4.59 (dtd, J=13.0, 8.0, 4.8 Hz, 1H), 4.03-3.69 (m, 1H), 3.36-2.92 (m, 2H), 2.52 (dd, J=4.8, 2.9 Hz, 1H), 2.13-1.92 (m, 6H). ¹⁹F NMR (376 MHz, DMSO-d₆) δ −61.20, −74.95. Insufficient quantity for ¹³C NMR. MS (ESI⁺): for C₁₇H₂₁F₃N₃O₃³²S [M+H]⁺ requires 404.13 found 404.13.

W-4-CF₃

¹H NMR (500 MHz, DMSO-d₆) δ 12.89 (s, 1H), 11.59 (d, J=2.6 Hz, 1H), 8.87 (d, J=7.8 Hz, 1H), 8.10 (s, 3H), 7.71 (d, J=8.1 Hz, 1H), 7.43 (d, J=7.2 Hz, 1H), 7.40 (d, J=2.6 Hz, 1H), 7.24 (t, J=7.8 Hz, 1H), 4.59 (dtd, J=13.0, 8.0, 4.8 Hz, 1H), 4.03-3.69 (m, 1H), 3.12 (ddd, J=40.7, 15.3, 9.3 Hz, 2H), 2.52 (dd, J=4.8, 2.9 Hz, 1H), 2.13-1.92 (m, 6H). ¹⁹F NMR (376 MHz, DMSO-d₆) δ −57.61, −74.95. Insufficient quantity for ¹³C NMR. MS (ESI⁺): for C₁₇H₂₁F₃N₃O₃³²S [M+H]⁺ requires 404.13 found 404.13.

MetTyr[ortho-CF₃]

### (S)-2-((S)-2-amino-4-(methylthio)butanamido)-3-(4-hydroxy-3-(trifluoromethyl)phenyl)propanoic acid

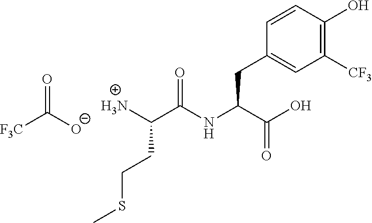 [SMILES: O=C([O-])C(F)(F)F ; CSCC[C@H]([NH3+])C(=O)N[C@@H](Cc1ccc(O)c(C(F)(F)F)c1)C(=O)O]

Synthesised following the general trifluoromethylation procedure to obtain a white solid after lyophilization (4.9 mg, 0.00981 mmol, 10%). Reverse phase HPLC details: Phenomenex Synergi™ 4 μm Hydro RP 80 Å, 250×10 mm. Flow rate=4 mL/min. Column temperature=40° C. Eluent=5% MeCN (0.1% TFA) and 85% H₂O (0.1% TFA) for 3 mins then increase linearly to 30% MeCN at 30 mins. Retention time=24.9 mins. ¹H NMR (500 MHz, DMSO-d₆) δ 12.99 (s, 1H), 10.51 (s, 1H), 8.76 (d, J=7.6 Hz, 1H), 8.16 (s, 3H), 7.39 (d, J=2.2 Hz, 1H), 7.33 (dd, J=8.5, 2.2 Hz, 1H), 6.95 (d, J=8.3 Hz, 1H), 4.53-4.33 (m, 1H), 3.83 (d, J=7.3 Hz, 1H), 3.05 (dd, J=14.2, 4.9 Hz, 1H), 2.89 (dd, J=14.2, 9.1 Hz, 1H), 2.49 (s, 2H), 2.04 (s, 5H). ¹³C NMR (126 MHz, DMSO-d₆) δ 172.27, 168.32, 154.56, 134.35, 127.33, 127.09 (q, J=4.7 Hz), 124.09 (q, J=272.0 Hz), 116.92, 115.10 (q, J=29.7 Hz), 53.98, 51.45, 35.18, 31.10, 27.81, 14.42. ¹⁹F NMR (470 MHz, DMSO-d₆) δ−62.34, −74.95. HRMS (ESI⁺): for C₁₅H₂₀F₃N₂O₄³¹S [M+H]⁺ requires 381.1090 found 381.1088. [α]_{D}²⁵=483° (c 0.073, H₂O).

Major Trifluoromethylated Side Products of Met-Tyr Reaction

### (S)-1-(((S)-1-carboxy-2-(4-hydroxy-2-(trifluoromethyl)phenyl)ethyl)amino)-4-(methylthio)-1-oxobutan-2-aminium

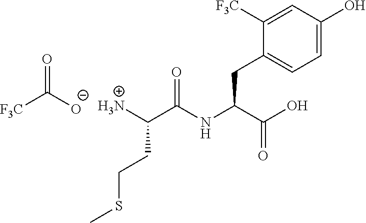 [SMILES: O=C([O-])C(F)(F)F ; CSCC[C@H]([NH3+])C(=O)N[C@@H](Cc1ccc(O)cc1C(F)(F)F)C(=O)O]

Synthesized following the general trifluoromethylation procedure 2: with 0.04 mmol of 70% aqueous TBHP (15 μL of a 2.67M stock solution in 25 mM aq. NH₄HCO₂), reaction time was 50 mins. The crude reaction mixture was diluted with about 2 mL of 10% MeCN (aq) with 0.1% TFA and each time 0.5 mL was injected into the HPLC loop (1 mL) for purification. The yield is not determined as the amount of product is not enough to be measured accurately on an analytical balance (≤0.1 g). Reverse phase HPLC details: Phenomenex Synergi™ 4 μm Hydro RP 80 Å, 250×10 mm. Flow rate=4 mL/min. Column temperature=40° C.

Eluent=5% MeCN (0.1% TFA) and 95% H₂O (0.1% TFA) for 3 mins then increase linearly to 30% MeCN at 30 mins. Retention time=24.3 mins. ¹H NMR (500 MHz, DMSO-d₆) δ 8.86 (d, J=8.2 Hz, 1H), 8.22-7.95 (m, 3H), 10.00 (s, 1H), 7.30 (d, J=8.4 Hz, 1H), 7.05 (d, J=2.6 Hz, 1H), 6.96 (dd, J=8.5, 2.7 Hz, 1H), 4.45 (ddd, J=9.6, 8.1, 5.4 Hz, 1H), 3.88-3.77 (m, 1H), 3.22 (dd, J=15.0, 5.5 Hz, 1H)partially obscured by HDO peak, 2.94 (dd, J=14.7, 9.8 Hz, 1H), 2.52-2.51 (m, 2H) overlap with solvent peak, 2.06 (s, 3H), 2.05-1.94 (m, 2H). ¹⁹F NMR (470 MHz, DMSO-d₆) δ −59.95, −74.95. Insufficient quantity for ¹³C NMR. MS (ESI⁺): for C₁₅H₂₀F₃N₂O₄³²S [M+H]⁺ requires 381.11 found 381.11.

### (2S)-1-(((S)-1-carboxy-2-(4-hydroxy-3-(trifluoromethyl)phenyl)ethyl)amino)-4-(methylsulfinyl)-1-oxobutan-2-aminium trifluoroacetate

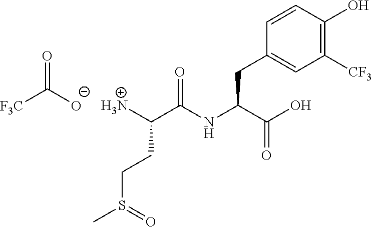 [SMILES: O=C([O-])C(F)(F)F ; CS(=O)CC[C@H]([NH3+])C(=O)N[C@@H](Cc1ccc(O)c(C(F)(F)F)c1)C(=O)O]

Synthesized following the general trifluoromethylation procedure 2: with 0.04 mmol of 70% aqueous TBHP (15 μL of a 2.67M stock solution in 25 mM NH₄HCO₂), reaction time was 50 mins. The crude reaction mixture was diluted with about 2 mL of 10% MeCN (aq) with 0.1% TFA and each time 0.5 mL was injected into the HPLC loop (1 mL) for purification. The yield is not determined as the amount of product is not enough to be measured accurately on an analytical balance (≤0.1 g). Reverse phase HPLC details: Phenomenex Synergi™ 4 μm Hydro RP 80 Å, 250×10 mm. Flow rate=4 mL/min. Column temperature=40° C. Eluent=5% MCCN (0.1% TFA) and 95% H₂O (0.1% TFA) for 3 mins then increase linearly to 30% MCCN at 30 mins. Retention time=21.3 mins. ¹H NMR (500 MHz, DMSO-d₆) δ 10.41 (s, 1H), 8.76 (s, 1H), 7.39 (s, 1H), 7.32 (d, J=8.3 Hz, 1H), 6.94 (d, J=8.4 Hz, 1H), 4.46 (q, J-8.8, 7.4 Hz, 1H), 3.89 (s, 1H), 3.05 (dd, J=14.2, 4.9 Hz, 1H), 2.97-2.85 (m, 2H), 2.86-2.72 (m, 1H), 2.54 (s, 3H)partially obscured by solvent peak, 2.15-1.93 (m, 2H). Cosy does not provide correlation, probably due to exchange at the NHs group. Carboxylic acid proton and NH₃ protons were not observed. ¹⁹F NMR (471 MHz, DMSO-d₆) δ −62.32, −62.33, −74.95. CFs is observed as two singlets of similar chemical shifts probably due to the new chiral centre at the sulfoxide. MS (ESI⁺): for C₁₅H₂₀F₃N₂O₅³²S [M+H]⁺ requires 397.10 found 397.10.

### Tyr[ortho-CFs]His

### 4-((S)-2-((S)-2-ammonio-3-(4-hydroxy-3-(trifluoromethyl)phenyl)propanamido)-2-carboxyethyl)-1H-imidazol-3-ium trifluoroacetate

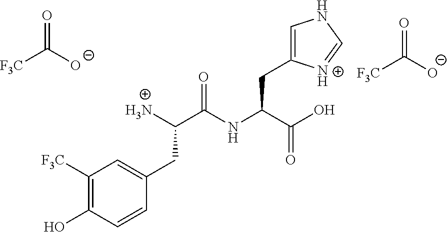 [SMILES: O=C([O-])C(F)(F)F ; [NH3+][C@@H](Cc1ccc(O)c(C(F)(F)F)c1)C(=O)N[C@@H](Cc1c[nH]c[nH+]1)C(=O)O ; O=C([O-])C(F)(F)F]

Synthesised following the general trifluoromethylation procedure to obtain a white solid after lyophilization (7.0 mg, 0.0114 mmol, 11%). Reverse phase HPLC details: Phenomenex Synergi™ 4 μm Hydro RP 80 Å, 250×10 mm. Flow rate=4 mL/min. Column temperature=40° C. Eluent=5% MeCN (0.1% TFA) and 95% H₂O (0.1% TFA) for 3 mins then increase linearly to 25% MeCN at 25 mins. Retention time=17.3 mins. ¹H NMR (500 MHz, DMSO-d₆) δ 14.41 (s, 2H), 10.58 (s, 1H), 9.01 (d, J=8.1 Hz, 2H), 8.10 (s, 3H), 7.43 (t, J=2.8 Hz, 2H), 7.32 (dd, J=8.5, 2.2 Hz, 1H), 6.95 (d, J=8.4 Hz, 1H), 4.65 (td, J=8.1, 5.2 Hz, 1H), 4.02 (t, J=6.4 Hz, 1H), 3.20 (dd, J=15.2, 5.3 Hz, 1H), 3.07 (td, J=12.7, 10.8, 6.7 Hz, 2H), 2.88 (dd, J=14.4, 8.3 Hz, 1H). Carboxylic acid OH is not observed. ¹³C NMR (126 MHz, DMSO-d₆) δ 171.38, 168.22, 155.08, 134.89, 134.04, 128.94, 127.65 (q, J=4.3, 3.8 Hz), 124.62, 124.05 (q, J=272.5 Hz), 117.12, 117.03, 115.42 (q, J=30.0 Hz), 53.33, 51.36, 35.73, 26.32. ¹⁹F NMR (470 MHz, DMSO-d₆) δ −62.22, −74.95. HRMS (ESI⁺): for C₁₆H₁₈O₄N₄F₃[M+H]⁺ requires 387.1275 found 387.1278. [α]_{D}²⁵=+28±1° (c 0.073, H₂O).

Major Trifluoromethylated Side Products of Tyr-His Reactions

### 4-((S)-2-((S)-2-ammonio-3-(4-hydroxy-2-(trifluoromethyl)phenyl)propanamido)-2-carboxyethyl)-1H-imidazol-3-ium

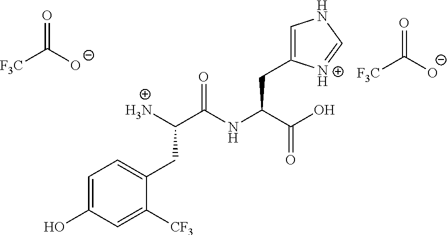 [SMILES: O=C([O-])C(F)(F)F ; [NH3+][C@@H](Cc1ccc(O)cc1C(F)(F)F)C(=O)N[C@@H](Cc1c[nH]c[nH+]1)C(=O)O ; O=C([O-])C(F)(F)F]

Synthesized following the general trifluoromethylation procedure 2: with 0.08 mmol of 70% aqueous TBHP (30 μL of a 2.67M stock solution in 25 mM NH₄HCO₂), reaction time was 50 mins. The crude reaction mixture was diluted with about 2 mL of 5% MeCN (aq) with 0.1% TFA and each time 0.5 mL was injected into the HPLC loop (1 mL) for purification. The yield is not determined as the amount of product is not enough to be measured accurately on an analytical balance (≤0.1 g). Reverse phase HPLC details: Phenomenex Synergi™ 4 μm Hydro RP 80 Å, 250×10 mm. Flow rate=4 mL/min. Column temperature=40° C. Eluent=5% MCCN (0.1% TFA) and 95% H₂O (0.1% TFA) for 3 mins then increase linearly to 25% MCCN at 25 mins. Retention time=16.1 mins. ¹H NMR (500 MHz, DMSO-d₆) δ 10.07 (s, 1H), 8.75 (d, J=7.9 Hz, 1H), 8.28 (s, 3H), 7.25 (d, J=8.5 Hz, 1H), 7.05 (d, J=2.6 Hz, 1H), 6.96 (dd, J=8.4, 2.6 Hz, 1H), 5.32 (t, J=5.0 Hz, 1H), 4.60 (q, J=7.3 Hz, 1H), 3.17-2.92 (m, 41H). Imidazole's proton (3H) and CO₂H (1H) were not observed. ¹⁹F NMR (471 MHz, DMSO-d₆) δ −59.68, −74.95. Insufficient quantity for ¹³C NMR. HRMS (ESI⁺): for C₁₆H₁₈O₄N₄F₃ [M+H]⁺ requires 387.1275 found 387.1270.

HisTrp[W-2-CF₃]

### 5-((S)-2-ammonio-3-(((S)-1-carboxy-2-(2-(trifluoromethyl)-1H-indol-3-yl)ethyl)amino)-3-oxopropyl)-1H-imidazol-3-ium trifluoroacetate

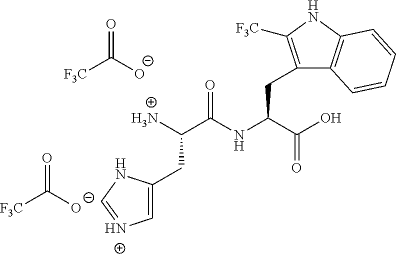 [SMILES: O=C([O-])C(F)(F)F ; O=C([O-])C(F)(F)F ; [NH3+][C@@H](Cc1c[nH+]c[nH]1)C(=O)N[C@@H](Cc1c(C(F)(F)F)[nH]c2ccccc12)C(=O)O]

Synthesised following the general trifluoromethylation procedure to obtain a white solid (7.4 mg, 0.0116 mmol, 12%). Reverse phase HPLC details: Phenomenex Synergi™ 4 μm Hydro RP 80 Å, 250×10 mm. Flow rate=4 mL/min. Column temperature=40° C. Eluent=11% MCCN (0.1% TFA) and 89% H₂O (0.1% TFA) for 3 mins then increase linearly to 25% MeCN at 25 mins, then another linear increase to 40% at 30 mins. Retention time=20.6 mins. ¹H NMR (500 MHz, DMSO-d₆) δ 14.39 (s, 2H), 12.07 (s, 1H), 9.02-8.97 (m, 2H), 8.38-8.33 (m, 3H), 7.77 (d, J=8.1 Hz, 1H), 7.45 (d, J=8.3 Hz, 1H), 7.42 (s, 1H), 7.32-7.24 (m, 1H), 7.14 (t, J=7.5 Hz, 1H), 4.56 (td, J=8.1, 6.7 Hz, 1H), 4.14 (t, J=6.7 Hz, 1H), 3.31 (dd, J=14.6, 8.3 Hz, 1H), 3.27-3.20 (m, 2H), 3.16 (dd, J=15.6, 7.3 Hz, 1H).

Carboxylic acid OH is not observed. ¹³C NMR (126 MHz, DMSO-d₆) δ 172.10, 167.29, 135.69, 134.45, 126.71, 124.39, 122.17 (q, J=268.9 Hz), 121.70 (q, J=36.1 Hz), 120.14, 119.94, 118.00, 112.43, 112.01 (q, J=2.9 Hz), 53.59, 51.22, 26.45, 26.37. ¹⁹F NMR (470 MHz, DMSO-d₆) δ −57.37, −74.95. HRMS (ESI⁺): for C₁₈H₁₉F₃N₅O₃[M+H]^{˜} requires 410.1434 m/z found 410.1429 m/z. [α]_{D}²⁵=+10±2° (c 0.119, H₂O).

Major Trifluoromethylated Side Products of his-Trp Reactions

### 5-((S)-2-ammonio-3-(((S)-1-carboxy-2-(4-(trifluoromethyl)-1H-indol-3-yl)ethyl)amino)-3-oxopropyl)-1H-imdazol-3-ium trifluoroacetae and 5-((S)-2-ammonio-3-(((S)-1-carboxy-2-(7-(trifluoromethyl)-1H-indol-3-yl)ethyl)amino)-3-oxopropyl)-1H-imidazol-3-ium trifluoroacetate (Not Separated)

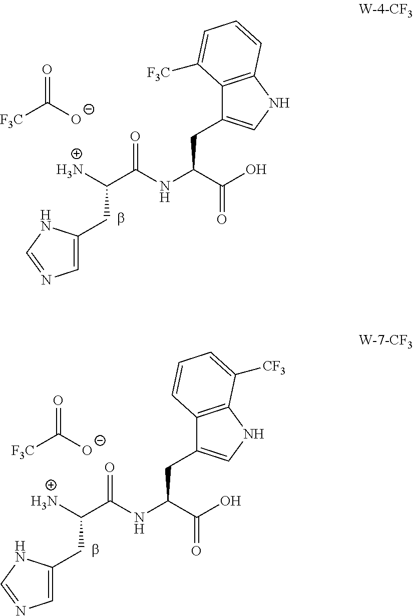 [SMILES: O=C([O-])C(F)(F)F ; [NH3+][C@@H](Cc1cnc[nH]1)C(=O)N[C@@H](Cc1c[nH]c2cccc(C(F)(F)F)c12)C(=O)O ; [NH3+][C@@H](Cc1cnc[nH]1)C(=O)N[C@@H](Cc1c[nH]c2c(C(F)(F)F)cccc12)C(=O)O ; O=C([O-])C(F)(F)F]

Synthesized following the general trifluoromethylation procedure 2: with 0.08 mmol of 70% aqueous TBHP (30 μL of a 2.67M stock solution in 25 mM NH₄HCO₂), reaction time was 50 mins. The crude reaction mixture was diluted with about 2 mL of 5% MCCN (aq) with 0.1% TFA and each time 0.5 mL was injected into the HPLC loop (1 mL) for purification. The yield is not determined as the amount of product is not enough to be measured accurately on an analytical balance (≤0.1 g). Reverse phase HPLC details: Phenomenex Synergi™ 4 μm Hydro RP 80 Å, 250×10 mm. Flow rate=4 mL/min. Column temperature=40° C. Eluent=11% MCCN (0.1% TFA) and 89% H₂O (0.1% TFA) for 3 mins then increase linearly to 25% MeCN at 25 mins, then another linear increase to 40% at 30 mins. Retention time=28.1 mins.

W-4-CF₃

¹H NMR (500 MHz, DMSO-d₆) δ 11.58 (d, J=2.7 Hz, 1H), 9.01 (s, 1H), 8.18 (s, 3H), 7.71 (d, J=8.2 Hz, 1H), 7.43 (d, J=7.3 Hz, 1H), 7.40 (d, J=2.6 Hz, 1H), 7.24 (t, J=7.8 Hz, 1H), 4.60 (d, J=8.4 Hz, 1H), 4.07 (s, 1H), 3.28-2.94 (m, 2H). Imidazole's protons are not observed. 8 protons of Histidine are not observed. Carboxylic acid proton is not observed. Insufficient quantity for ¹³C NMR. ¹⁹F NMR (470 MHz, DMSO-d₆) δ −57.59, −74.95. HRMS (ESI⁺): for C₁₅H₁₉F₃N₅O₃[M+H]⁺ requires 410.1434 m/z found 410.1452 m/z.

W-7-CF₃

¹H NMR (500 MHz, DMSO-d₆) δ 11.37-11.26 (m, 1H), 9.01 (s, 2H), 8.18 (s, 3H), 7.87 (d, J=8.0 Hz, 1H), 7.44 (d, J=6.9 Hz, 1H), 7.31 (d, J=2.5 Hz, 1H), 7.18 (t, J=7.7 Hz, 1H), 4.65-4.50 (m, 1H), 4.07 (s, 1H), 3.27-2.96 (m, 2H). Imidazole's protons are not observed. β protons of Histidine are not observed. Carboxylic acid proton is not observed. Insufficient quantity for ¹³C NMR. ¹⁹F NMR (470 MHz, DMSO-d₆) δ −61.18, −74.95. HRMS (ESI⁺): for C₁₈H₁₉F₃N₅O₃ [M+H]⁺ requires 410.1434 m/z found 410.1452 m/z.

GluTrp[W-2-CF₃]

### (S)-4-carboxy-1-(((S)-1-carboxy-2-(2-(trifluoromethyl)-1H-indol-3-yl)ethyl)amino)-1-oxobutan-2-aminium trifluoroacetate

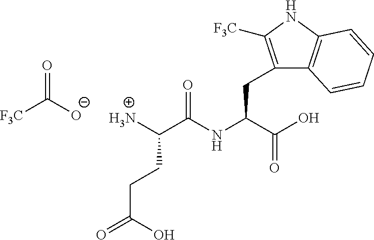 [SMILES: O=C([O-])C(F)(F)F ; [NH3+][C@@H](CCC(=O)O)C(=O)N[C@@H](Cc1c(C(F)(F)F)[nH]c2ccccc12)C(=O)O]

To GluTrp (10.4 mg, 31 μmol) in a 3 mL screw top V-Vials® with open-top cap (Sigma-Aldrich Z115142) was added 90 μL of DMSO, Iron (III) Chloride (5.5 mg, 34 μmol), NaSO₂CF₃ (45 μmol, 45 μL of a 1M stock solution in 25 mM aqueous NH₄HCO₂), and 70% TBHP in water (84 μmol, 45 μL of a 1.87M stock solution in 25 mM aqueous NH₄HCO₂). The vial was placed in a water bath at 40° C. and stirred at 760 rpm for 30 mins. The crude reaction mixture was diluted with about 2 mL of 5% MeCN (aq) with 0.1% TFA and each time 0.5 mL was injected into the HPLC loop (1 mL) for purification. A white solid which slowly turns brown at −20° C. was obtained after lyophilization (4.6 mg, 3.4 μmol, 11%). Reverse phase HPLC details: Phenomenex Synergi™ 4 μm Hydro RP 80 Å, 250×10 mm. Flow rate=4 mL/min. Column temperature=40° C. Eluent=12% MeCN (0.1% TFA) and 88% H₂O (0.1% TFA) for 3 mins then increase linearly to 40% MeCN at 25 mins. Retention time=17.7 mins. ¹H NMR (500 MHz, DMSO-d₆) δ 12.90 (s, 1H), 12.05 (s, 1H), 8.94 (d, J=8.0 Hz, 1H), 8.12 (d, J=5.3 Hz, 3H), 7.76 (d, J=8.1 Hz, 1H), 7.44 (d, J=8.3 Hz, 1H), 7.29 (t, J=7.6 Hz, 1H), 7.14 (t, J=7.5 Hz, 1H), 4.54 (q, J=7.6 Hz, 1H), 3.78 (q, J=5.7 Hz, 1H), 3.34 (dd, J=14.5, 7.9 Hz, 1H), 3.22 (dd, J=14.8, 7.1 Hz, 1H), 2.38-2.31 (m, 2H), 1.98 (dt, J=9.4, 6.7 Hz, 2H). ¹³C NMR (126 MHz, DMSO-d₆) δ 173.38, 172.18, 168.29, 158.85-156.88 (m), 135.63, 126.73, 124.35, 122.15 (q, J=268.7 Hz), 121.62 (q, J=36.1 Hz), 120.12, 119.97, 116.73 (q, J=297.1 Hz), 112.36, 112.15 (q, J=2.7 Hz), 53.50, 51.45, 28.84, 26.33, 26.15. ¹⁹F NMR (471 MHz, DMSO-d₆) δ −57.32, −74.95. HRMS (ESI⁺): for C₁₇H₁₉O₅N₃F₃[M+H]⁺ requires 402.1271 m/z found 402.1268 m/z. [α]_{D}²⁵=+14±2° (c 0.040, H₂O).

Major Trifluoromethylated Side Products of Glu-Trp Reactions

### (S)-4-carboxy-1-(((S)-1-carboxy-2-(7-(trifluoromethyl)-1H-indol-3-yl)ethyl)amino)-1-oxobutan-2-aminium and (S)-4-carboxy-1-(((S)-1-carboxy-2-(4-(trifluoromethyl)-1H-indol-3-yl)ethyl)amino)-1-oxobutan-2-aminium (not separated)

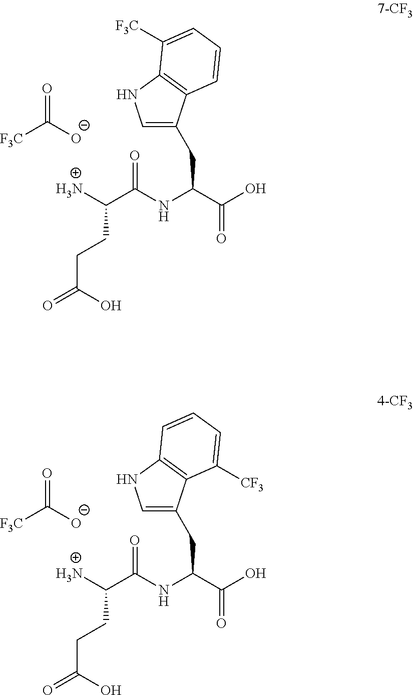 [SMILES: O=C([O-])C(F)(F)F ; [NH3+][C@@H](CCC(=O)O)C(=O)N[C@@H](Cc1c[nH]c2c(C(F)(F)F)cccc12)C(=O)O ; O=C([O-])C(F)(F)F ; [NH3+][C@@H](CCC(=O)O)C(=O)N[C@@H](Cc1c[nH]c2cccc(C(F)(F)F)c12)C(=O)O]

Reverse phase HPLC details: Phenomenex Synergi™ 4 μm Hydro RP 80 Å, 250×10 mm. Flow rate=4 mL/min. Column temperature=40° C. Eluent=12% MeCN (0.1% TFA) and 88% H₂O (0.1% TFA) for 3 mins then increase linearly to 40% MCCN at 25 mins. Retention time 21.4 mins. (4-CF₃ and 7-CF₃ are not separated).

4-CF₃

¹H NMR (500 MHz, DMSO-d₆) δ 12.33 (s, 1H), 11.64-11.51 (m, 1H), 8.87 (d, J=7.7 Hz, 1H), 8.09 (s, 3H), 7.71 (d, J=8.1 Hz, 1H), 7.43 (d, J=7.3 Hz, 1H), 7.41 (d, J=2.6 Hz, 2H), 7.24 (t, J=7.8 Hz, 1H), 4.69-4.46 (m, 1H), 3.82 (s, 1H), 3.48-2.99 (m, 2H), 2.07-1.91 (m, 4H). ¹⁹F NMR (471 MHz, DMSO-d₆) δ −57.66, −61.23, −74.95. Insufficient quantity for ¹³C NMR. HRMS (ESI⁺): for C₁₇H₁₉O₅N₃F₃[M+H]⁺ requires 402.1271 m/z found 402.1279 m/z.

7-CF₃

¹H NMR (500 MHz, DMSO-d₆) δ 12.88 (s, 2H), 11.32 (d, J=2.1 Hz, 1H), 8.76 (d, J=7.7 Hz, 1H), 8.09 (s, 3H), 7.87 (d, J=7.9 Hz, 1H), 7.45 (d, J=6.8 Hz, 1H), 7.32 (d, J=2.1 Hz, 1H), 7.18 (t, J=7.7 Hz, 1H), 4.69-4.46 (m, 1H), 3.82 (s, 1H), 3.36-2.99 (m, 2H), 2.07-1.91 (m, 4H). ¹⁹F NMR (471 MHz, DMSO−d₆) δ −57.66, −61.23, −74.95. Insufficient quantity for ¹³C NMR. HRMS (ESI⁺): for C₁₇H₁₉O₅N₃F₃[M+H]⁺ requires 402.1271 m/z found 402.1279 m/z.

Angiotensin(1-7)[Y-ortho-CF₃]

### 4-((6S,9S,12S,15S,18S)-1-amino-6-((S)-2-ammonio-3-carboxypropanamido)-15-((S)-sec-butyl)-18-((S)-2-carboxypyrrolidine-1-carbonyl)-12-(4-hydroxy-3-(trifluoromethyl)benzyl)-1-iminio-9-isopropyl-7,10,13,16-tetraoxo-2,8,11,14,17-pentaazanonadecan-19-yl)-1H-imidazol-3-ium trifluoroacetate

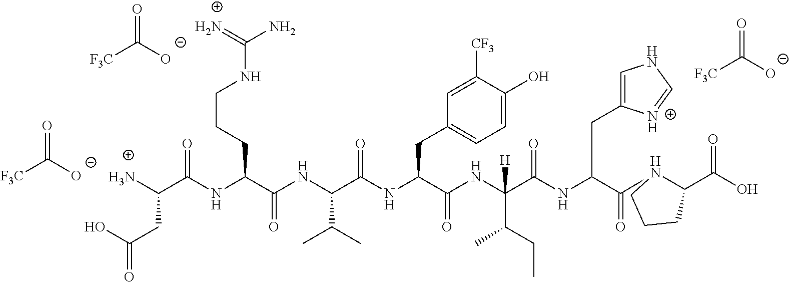

To Angiotensin (1-7) (6.3 mg, 5.1 μmol) in a 3 mL screw top V-Vials® with open-top cap (Sigma-Aldrich Z115142) was added 20 μL of DMSO, Iron (III) Chloride (4.4 mg, 27 μmol), NaSO₂CF₃ (10 μmol, 10 μL of a 1M stock solution in 25 mM aqueous NH₄HCO₂), and 70% TBHP in water (19 μmol, 10 μL of a 1.87M stock solution in 25 mM aqueous NH₄HCO₂). The vial was placed in a water bath at 40° C. and stirred at 760 rpm for 50 mins. The crude reaction mixture was diluted with about 2 mL of 10% MeCN (aq) with 0.1% TFA and each time 0.5 mL was injected into the HPLC loop (1 mL) for purification. A white solid was obtained after lyophilization (2.4 mg, 1.8 μmol, 36%). Reverse phase HPLC details: Phenomenex Synergi™ 4 μm Hydro RP 80 Å, 250×10 mm. Flow rate=4 mL/min. Column temperature=40° C. Eluent=8% MeCN (0.1% TFA) and 92% H₂O (0.1% TFA) for 3 mins then increase linearly to 30% MeCN at 30 mins. Retention time 25.9 mins. ¹H NMR (500 MHz, DMSO-d₆) Only Tyrosine residue protons are listed. 7.41 (d, J=2.1 Hz, 1H), 7.31 (dd, J=8.6, 2.1 Hz, 1H), 7.23 (s, 2H), ¹⁹F NMR (471 MHz, DMSO-d₆) δ −62.17, −74.95. HRMS (ESI⁺): for C₄₂H₆₂O₁₁N₁₂F₃[M+H]⁺ requires 967.4608 found 967.4606.

Major Trifluoromethylated Side Product of Angiotensin(1-7) Reaction

### Angiotensin(1-7)[meta-CF₃] 4-((6S,9S,12S,15S,18S)-1-amino-6-((S)-2-ammonio-3-carboxypropanamido)-15-((S)-sec-butyl)-18-((S)-2-carboxypyrrolidine-1-carbonyl)-12-(4-hydroxy-2-(trifluoromethyl)benzyl)-1-iminio-9-isopropyl-7,10,13,16-tetraoxo-2,8,11,14,17-pentaazanonadecan-19-yl)-1H-imidazol-3-ium trifluoroacetate

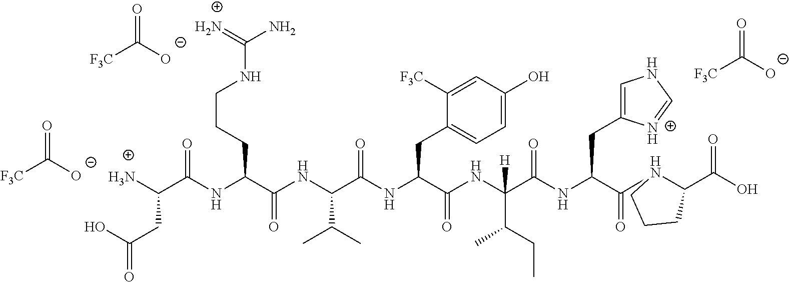

Reverse phase HPLC details: Phenomenex Synergi™ 4 μm Hydro RP 80 Å, 250×10 mm. Flow rate=4 mL/min. Column temperature=40° C. Eluent=8% MeCN (0.1% TFA) and 92% H₂O (0.1% TFA) for 3 mins then increase linearly to 30% MeCN at 30 mins. Retention time 25.9 mins. ¹⁹F NMR (471 MHz, DMSO-d₆) δ −60.03, −74.95. HRMS (ESI⁺): for C₄₂H₆₂O₁₁N₁₂F₃[M+H]⁺ requires 967.4608 found 967.4641.

Melittin[W-2-CF₃]

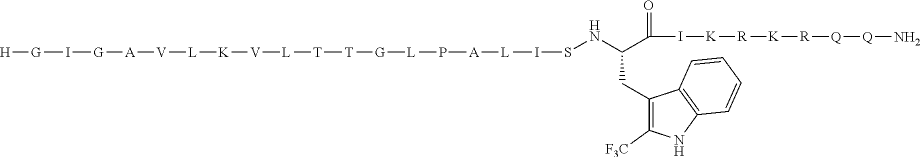

To Melittin (0.7 mg, 0.25 μmol) in a 3 mL screw top V-Vials® with open-top cap (Sigma-Aldrich Z115142) was added 25 μL of DMSO, Iron (III) Chloride (3.8 μmol, 5 μL of a 0.76M stock solution in 25 mM aqueous NH₄HCO₂), NaSO₂CF₃ (1.9 μmol, 5 μL of a 0.38M stock solution in 25 mM aqueous NH₄HCO₂), and 70% TBHP in water (3.5 μmol, 5 μL of a 0.70M stock solution in 25 mM aqueous NH₄HCO₂). Additional 10 μL of 25 mM NH₄HCO₂ (aq) was added. The vial was placed in a water bath at 40° C. and stirred at 760 rpm for 20 mins. About 1 mL of 15% MeCN (aq) with 0.1% TFA was added to quench the reaction for semi-preparative HPLC. The yield is not determined as the amount of product is not enough to be measured accurately on an analytical balance (≤0.1 g). Reverse phase HPLC details: Zorbax 300SB-C18, 9.4×250 mm 5p 300 Å. Flow rate=4 mL/min. Column temperature=40° C. Eluent=17.5% MeCN (0.1% TFA) and 82.5 water (0.1% TFA) for 6 mins and increase linearly to 55% at 25 mins. Retention time=23.3 mins. ¹H NMR (500 MHz, D₂O with 0.1% CF₃CO₂D) Only Trpytophan residue ¹H is listed, absence of singlet supported the position of CF₃. δ7.74 (d, J=8.0 Hz, 1H), 7.53 (d, J=8.6 Hz, 1H), 7.38 (t, J=8.1 Hz, 1H), 7.22 (t, J=7.6 Hz, 1H). ¹⁹F NMR (470 MHz, D₂O with 0.1% CF₃CO₂D) δ −57.87, −75.50. HRMS (ESI⁺): for C₁₃₂H₂₃₂O₃₁N₃₉F₃ [M+4H]⁴⁺ requires 729.1927 found 729.1926.

Somatostatin-14[W-2-CF₃]

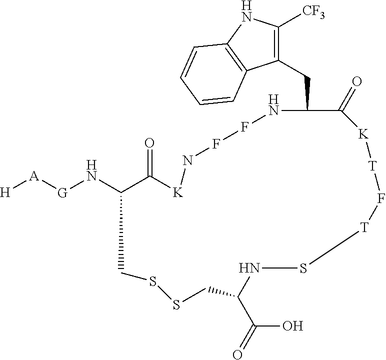

To Somatostatin-14 (2.4 mg, 1.5 μmol) in a 3 mL screw top V-Vials® with open-top cap (Sigma-Aldrich Z115142) was added 40 μL of DMSO, Iron (III) Chloride (7.6 μmol, 10 μL of a 0.76M stock solution in 25 mM aqueous NH₄HCO₂), NaSO₂CF₃ (5.7 μmol, 15 μL of a 0.38M stock solution in 25 mM aqueous NH₄HCO₂), and 70% TBHP in water (7.0 μmol, 10 μL of a 0.70M stock solution in 25 mM aqueous NH₄HCO₂). The vial was placed in a water bath at 40° C. and stirred at 760 rpm for 25 mins. About 1 mL of 15% MeCN (aq) with 0.1% TFA was added to quench the reaction for semi-preparative HPLC. The yield is not measured as the amount of product is not enough to be measured accurately on an analytical balance (≤0.1 g). Reverse phase HPLC details: Zorbax 300SB-C18, 9.4×250 mm 5p 300 Å. Flow rate=4 mL/min. Column temperature=40° C. Eluent=17.5% MeCN (0.1% TFA) in water (0.1% TFA) for 6 mins and increase linearly to 47% at 25 mins. Retention time=19.4 mins. ¹⁹F NMR (470 MHz, DMSO-d₆) δ −57.58, −74.95. HRMS (ESI⁺): for C₇₇H₁₀O₁₉N₁₉F₃³²S₂[M+H]⁺ requires 1705.7113 found 1705.7086.

Endomorphin 1[W-2-CF₃]

### (S)-1-((S)-2-(((S)-1-(((S)-1-carboxy-2-phenylethyl)amino)-1-oxo-3-(2-(trifluoromethyl)-1H-indol-3-yl)propan-2-yl)carbamoyl)pyrrolidin-1-yl)-3-(4-hydroxyphenyl)-1-oxopropan-2-aminium trifluoroacetate

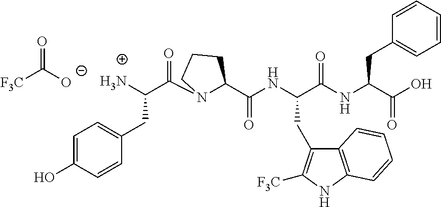 [SMILES: O=C([O-])C(F)(F)F ; [NH3+][C@@H](Cc1ccc(O)cc1)C(=O)N1CCC[C@H]1C(=O)N[C@@H](Cc1c(C(F)(F)F)[nH]c2ccccc12)C(=O)N[C@@H](Cc1ccccc1)C(=O)O]

To Endomorphin 1 (5.9 mg, 9.7 μmol) in a 3 mL screw top V-Vials® with open-top cap (Sigma-Aldrich Z115142) was added 40 μL of DMSO, Iron (III) Chloride (20 μmol, 20 μL of a 1M stock solution in 25 mM aqueous NH₄HCO₂), NaSO₂CF₃ (15 μmol, 15 μL of a 1M stock solution in 25 mM aqueous NH₄HCO₂), and 70% TBHP in water (15 μmol, 15 μL of a 1M stock solution in 25 mM aqueous NH₄HCO₂). The vial was placed in a water bath at 40° C. and stirred at 760 rpm for 40 mins. About 1 mL of 15% MCCN (aq) with 0.1% TFA was added to quench the reaction for semi-preparative HPLC. 0.59 mg (7% yield) of white solid was obtained after lyophilization. Reverse phase HPLC details: Synergi RP 4, 10.0×250 mm 5p 80 Å. Column temperature=40° C. Flow rate=4 mL/min. Eluent=17% MeCN (0.1% TFA) in water (0.1% TFA) for 3 mins and increase linearly to 48% at 25 mins. Retention time=19.8 mins. ¹H NMR (500 MHz, DMSO-d₆) δ 11.91 (s, 1H), 9.33 (s, 1H), 8.08 (d, J=8.1 Hz, 1H), 7.99 (s, 3H), 7.83 (d, J=8.1 Hz, 1H), 7.74 (d, J=8.1 Hz, 1H), 7.40 (d, J=8.2 Hz, 1H), 7.33-7.18 (m, 3H), 7.18-7.07 (m, 7H), 6.99 (s, 1H), 6.68 (d, J=8.5 Hz, 2H), 4.61-4.46 (m, 1H), 4.45-4.32 (m, 1H), 4.22 (s, 1H), 3.70-3.50 (m, 2H), 3.29-3.19 (m, 1H), 3.17-3.03 (m, 2H), 2.95 (td, J=14.7, 14.2, 5.8 Hz, 2H), 2.89-2.73 (m, 2H), 2.04-1.91 (m, 1H), 1.84-1.58 (m, 3H). Minor conformers peaks are not included. ¹⁹F NMR (470 MHz, DMSO-d₆) δ −57.51, −74.95. HRMS (ESI⁺): for C₃₅H₃₈O₅N₆F₃[M+H]⁺ requires 679.2850 found 679.2847.

NMR Assignment

Assignment of ¹H NMR is complicated by the presence of cis- and trans-conformer (28%/72%, determined from ¹H NMR by the integration of phenol OH's protons). This is also observed for Endomorphin 1 (cis- and trans-ratio of 25%/75%) as reported by Podlogar et al. (Podlogar, B. L.; Paterlini, M. G.; Ferguson, D. M.; Leo, G. C.; Demeter, D. A.; Brown, F. K.; Reitz, A. B. FEBS Lett. 1998, 439 (1-2), 13-20).

Major Trifluoromethylated Side Products of Endomorphin 1 Reaction

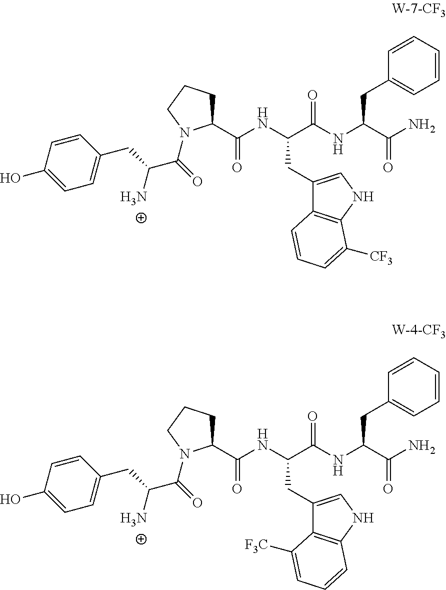 [SMILES: NC(=O)[C@H](Cc1ccccc1)NC(=O)[C@H](Cc1c[nH]c2c(C(F)(F)F)cccc12)NC(=O)[C@@H]1CCCN1C(=O)[C@H]([NH3+])Cc1ccc(O)cc1 ; NC(=O)[C@H](Cc1ccccc1)NC(=O)[C@H](Cc1c[nH]c2cccc(C(F)(F)F)c12)NC(=O)[C@@H]1CCCN1C(=O)[C@H]([NH3+])Cc1ccc(O)cc1]

Assignment of structure is based on ¹⁹F NMR chemical shift comparison with previous Trp-containing dipeptides and L-Tryptophan. TOCSY revealed coupling between indole NH and C2-H thus supporting that CF₃ is not on C2. MS/MS analysis. 4 indole's NHs are due to cis- and trans-isomer. ¹⁹F NMR (470 MHz, DMSO-d₆) δ −57.22, −57.24, −61.08, −61.10, −74.95. MS (ESI⁺): for C₃₅H₃₈O₅N₆F₃[M+H]⁺ requires 679.29 found 679.31.

Insulin

Synthesized according to the procedure of Krska et al(Ichiishi, N.; Caldwell, J. P.; Lin, M.; Zhong, W.; Zhu, X.; Streckfuss, E. C.; Kim, H.-Y. Y.; Parish, C. A.; Krska, S. W. Chem. Sci. 2018, 9 (17), 4168-4175) except that no precaution was taken to exclude oxygen and water. The reaction was done on a smaller scale of 20 mg of recombinant insulin. Purification was performed by reversed phase HPLC. Reverse phase HPLC details: Zorbax 300SB-C18, 9.4×250 mm 5μ 300 Å. Flow rate=4 mL/min. Column temperature=40° C. Eluent=24% MeCN (0.1% TFA) and 76% H₂O (0.1% TFA) for 3 mins and increase linearly to 42% MeCN at 25 mins.

Insulin Chain A Y14-CF₃

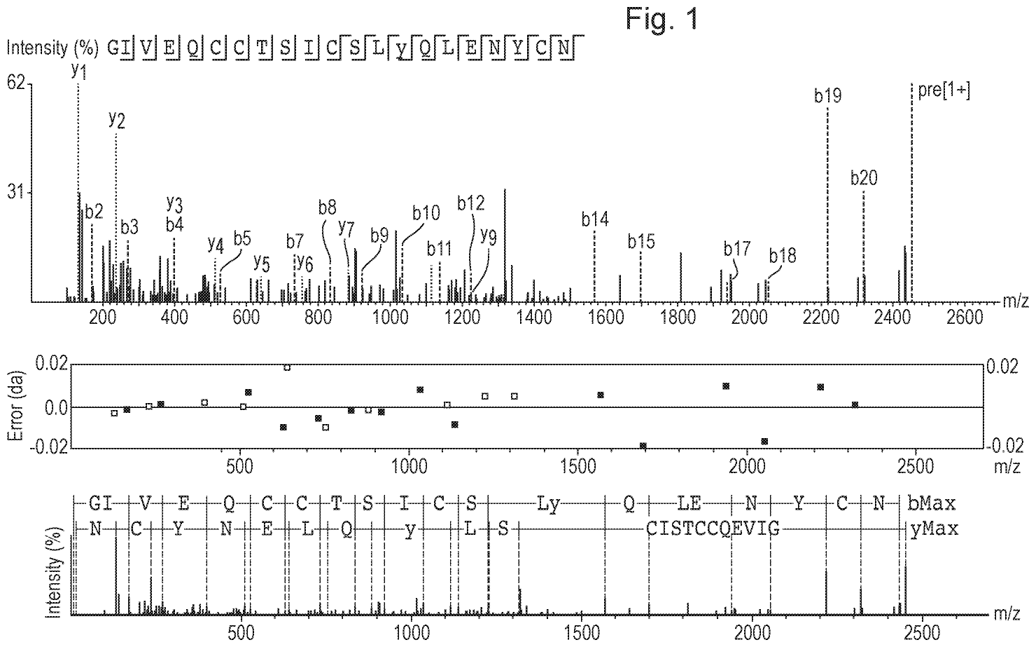

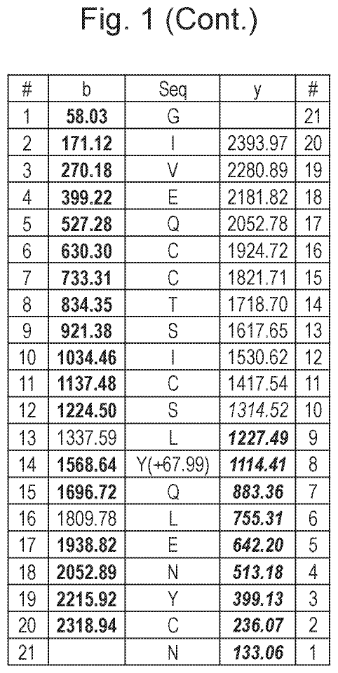

2.9 mg of white solid was obtained after lyophilization. Retention time=19.1 mins. ¹⁹F NMR (471 MHz, Deuterium Oxide+0.1% (v/v) Trifluoroacetic Acid-d₁) δ −62.06, −75.51. MS data is shown in FIG. 1. Y14: −10lgP=69.54, AScore 100.21

Insulin Chain A Y19-CF₃

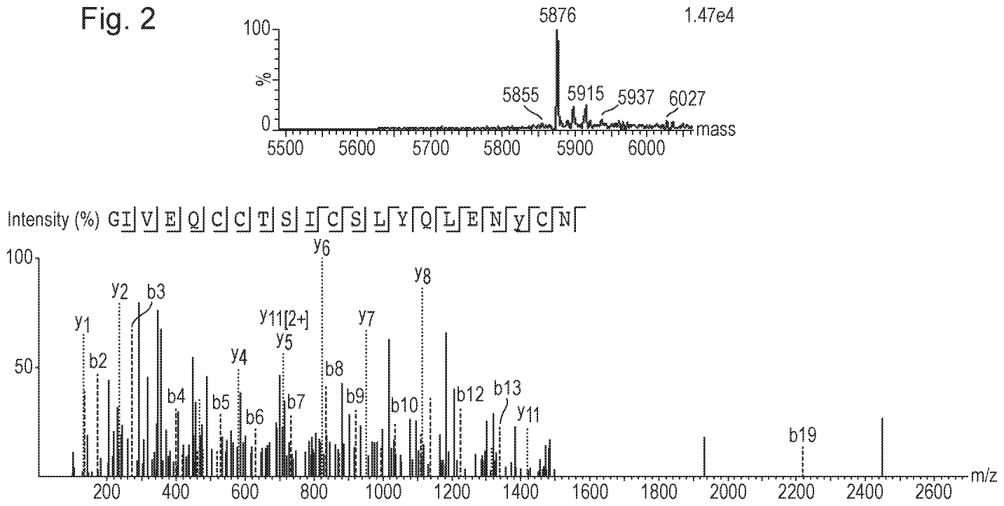

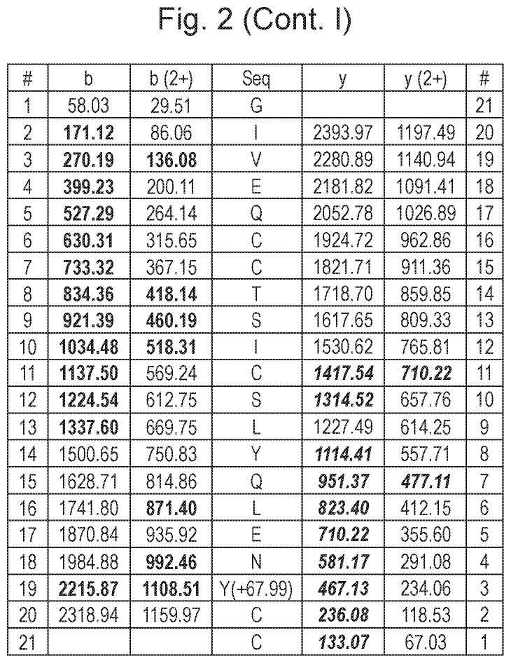

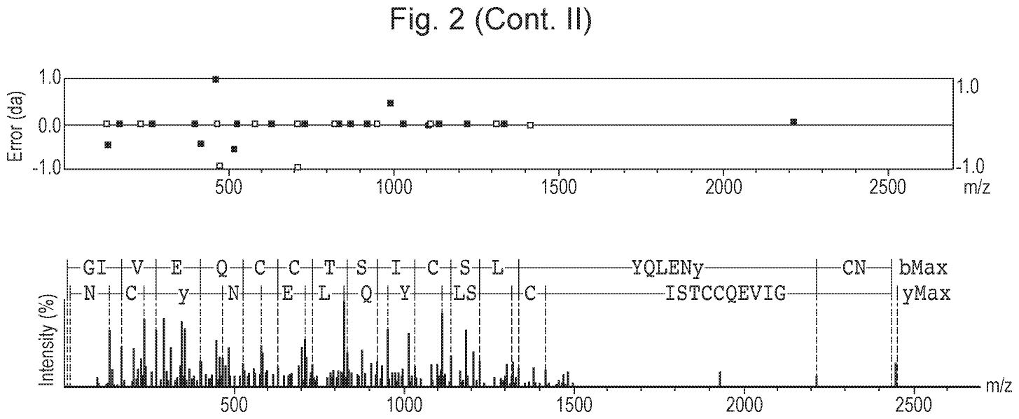

2.9 mg of white solid was obtained after lyophilization. Retention time=18.0 mins. ¹⁹F NMR (471 MHz, Deuterium Oxide+0.1% (v/v) Trifluoroacetic Acid-d₁) δ −60.91, −75.51. MS data is shown in FIG. 2. Modification found on Chain A: Y19: −10lgP=112.31, AScore=27.96

Insulin Chain B Y16-CF₃ and Insulin Chain B Y26-CF₃

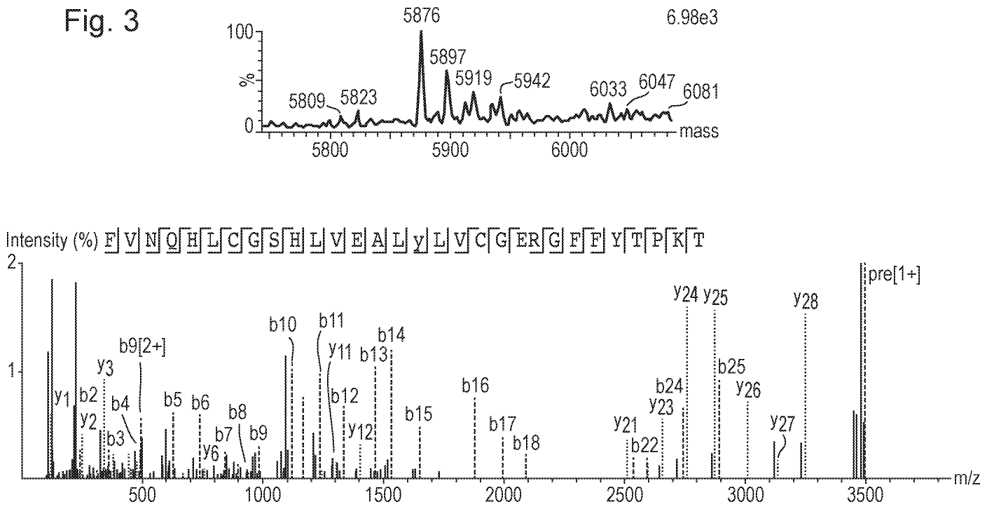

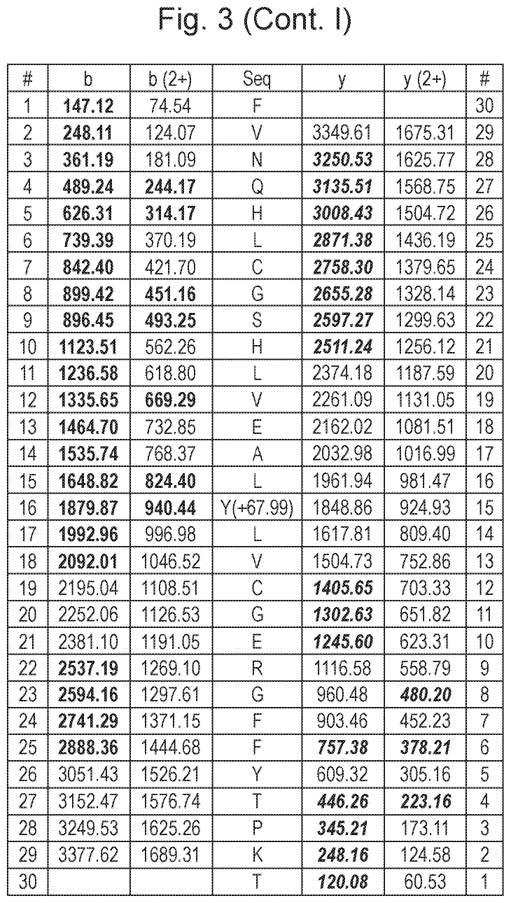

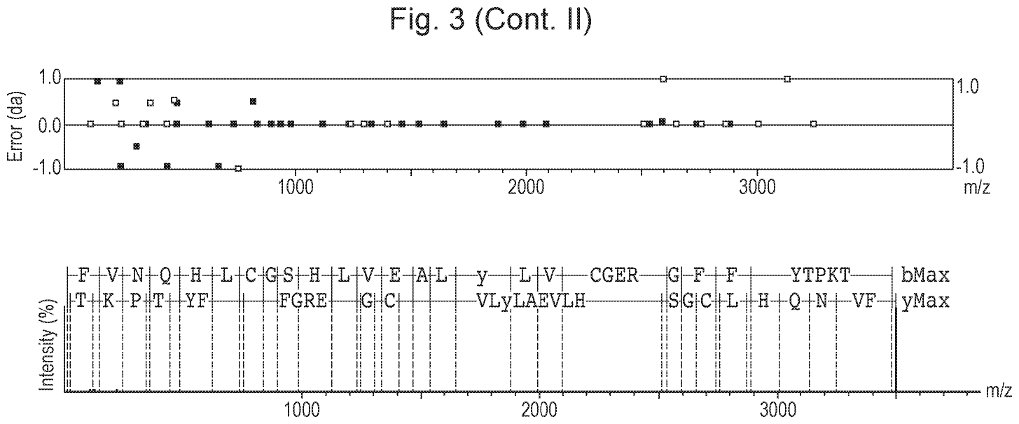

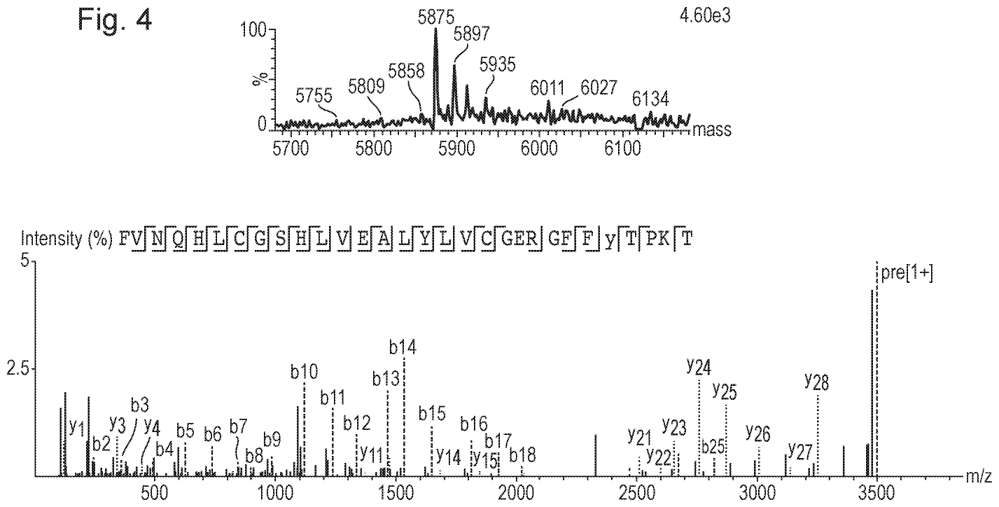

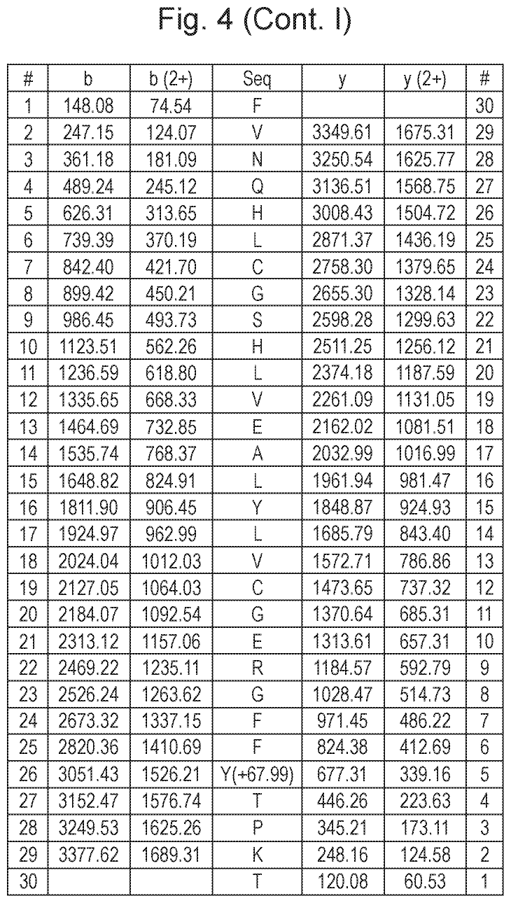

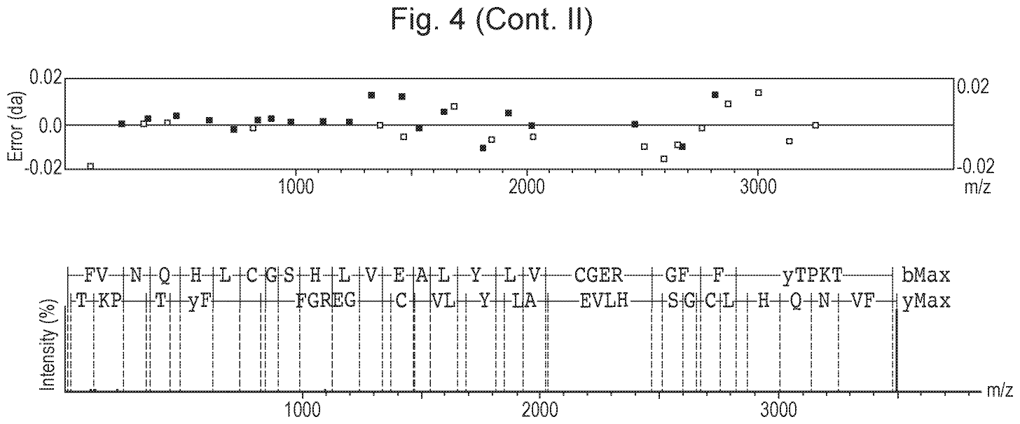

1.7 mg of white solid was obtained after lyophilization. Y16-CF₃ and Y26-CF₃ are separable to a certain extent, however, for convenience, they were collected together for NMR measurement. As LC-MS/MS requires a lower amount of analyte, a small amount of these were separately purified for LC-MS/MS. ¹⁹F NMR (471 MHz, Deuterium Oxide+0.1%(v/v) Trifluoroacetic Acid-d₁) δ −61.92, −61.95, −75.51. MS data is shown in FIGS. 3 and 4. Y16: −10 lgP=200.00, Ascore 40.63. Y26: −10 lgP=200.00, AScore 14.02

Radiochemistry

General Experimental Details

¹⁸F-Fluoride was produced by Alliance Medical (UK) via the ¹⁸O(p,n) ¹⁸F reaction and delivered as ¹⁸F-fluoride in ¹⁸O-water. Radiosynthesis and azeotropic drying was performed on a NanoTek® automated microfluidic device (Advion). HPLC analysis was performed with a Dionex Ultimate 3000 dual channel HPLC system equipped with shared autosampler, parallel UV-detectors and LabLogic NaI/PMT-radiodetectors with Flowram analog output. Radio-TLC was performed on Merck Kiesegel 60 F254 plates. Analysis was performed using a plastic scintillator/PMT detector. Mass-spec analysis (ESI) of crude reaction mixtures was performed by diverting part of the HPLC flow after passing through the radiodetector to the inlet of an Advion CMS. This enabled the detection of non-labelled ¹⁹F-compounds formed during the reaction (the amount of ¹⁸F-labelled compounds formed is below the limit of detection). Due to the separation of the modules the radio-signal is offset by 0.1-0.3 min from the UV signal.

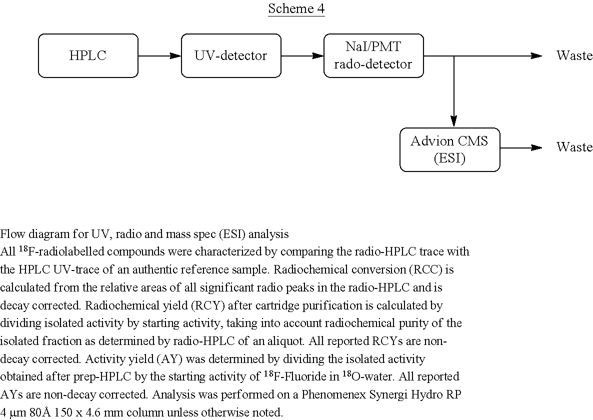

HPLC Eluents—Eluent A

HPLC gradient: MeCN/H₂O 25 mM NH₄HCO₂, 1 mL/min, Synergi 4 μm Hydro-RP 80 Å column, 150×4.6 mm

0-1 min (1% MeCN) isocratic

1-10 min (1% MeCN to 95% MeCN) linear increase

10-14 min (95% MeCN) isocratic

14-16 min (95% MeCN to 1% MeCN) linear decrease

16-18 min (5% MeCN) isocratic

Sep-Pak Cartridges Used for Purification

Waters Sep-Pak Al₂O₃ N light cartridge (part #WAT023561), Waters Sep-Pak SiO₂ light cartridge (part #WAT023537), Waters Sep-Pak SiO₂ plus cartridge (part #WAT020520), Waters Sep-Pak Dry Sodium Sulfate cartridge (part #WAT054265), Oasis MCX Plus cartridge (Waters, part #186003516), Oasis HLB Plus cartridge (Waters, part #186000132), Oasis HLB Light cartridge (Waters, part #186005125), Oasis MAX Plus cartridge (Waters, part #186003517). All cartridges were preconditioned with MeOH (2 mL) followed by H₂O (10 mL) unless otherwise indicated.

General Procedure for the Small Scale ¹⁸F-Labelling of [¹⁸F]CF₃SO₂NH₄

[¹⁸F]Fluoride (3.0-4.0 GBq) was separated from ¹⁸O-enriched-water using a Chromafix PSHCO₃ ¹⁸F separation cartridge (45 mg, activated by slowly passing through 1 mL of H₂O) and subsequently released with 900 μL (in 6×150 μL portions) of the K₂₂₂/K₂CO₃ solution into a 5 mL V-vial containing a magnetic stir bar in the concentrator. The solution was dried with five cycles of azeotropic drying with anhydrous MeCN (5×200 μL) under a flow of N₂ at 105° C. The dried [¹⁸F]KF/K₂₂₂ residue was re-dissolved in anhydrous DMF (1000 μL). A solution of [¹⁸F]KF/K₂₂₂ in DMF (20-30 MBq, 10-50 μL) was dispensed into a V-vial containing difluorocarbene reagent, amine-SO2 adduct and a magnetic stirrer bar. Anhydrous solvent (300 μL) was added via syringe before stirring at the specified temperature and time (See Table 1 to Table 4). The reaction was quenched with 10% EtOH in H₂O (200 μL). An aliquot was removed for analysis by radioTLC and radioHPLC. Analysis was performed using the gradient given below with an analytical Synergi 4 μm Hydro-RP 80 A column, 150×4.6 mm at a flow rate 1 mL/min. Radio-TLC was performed on Merck Kiesegel 60 F254 plates, using MeCN:MeOH:H₂O:AcOH 20:5:5:1 as eluent. Analysis was performed using a plastic scintillator/PMT detector. Radiochemical conversions are calculated from radioTLC and radioHPLC:

### Radiochemical Conversion (%)=RadioTLC Yield (%)×RadioHPLC Yield (%)

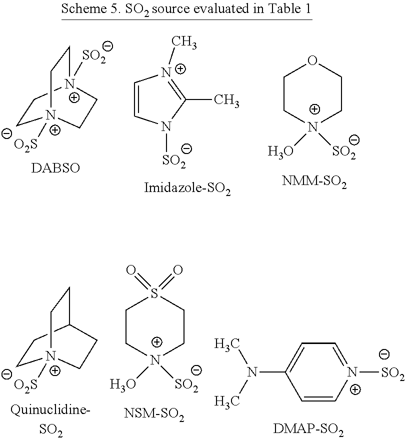

TABLE 1
Results for small-scale screening.

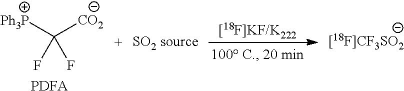

| Entry | SO₂ source^{a} | Solvent | RCC(%)^{b} |
|---|---|---|---|
| 1 | SO₂ | DMF | 0 |
| 2 | DABSO-SO₂ | DMF | 0 |
| 3 | NMM-SO₂ | DMF | 20 ± 10^{c} |
| 5 | NMM-SO₂ | NMP | 12 ± 1 |
| 6 | Quinuclidine-SO₂ | NMP | 4 ± 2 |
| 7 | DMAP-SO₂ | NMP | 2 ± 1 |
| 8 | Imidazole-SO₂ | NMP | 4 ± 0 |
| 9 | NSM-SO₂ | NMP | 4 ± 2 |

General conditions: 0.080 mmol of PDFA, 0.021 mmol of SO₂ source and 300 μL of solvent.
^{a}Refer to Scheme 4.
^{b}Based-on n = 2.
^{c}Based-on n = 4.

TABLE 2
Variation of stoichiometry, reaction time and temperature with NMM-SO₂
for small-scale screening

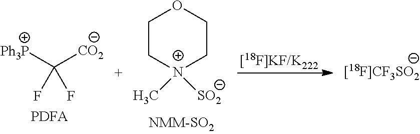

| Entry | NMM-SO₂(mmol) | PDFA(mmol) | Time(min) | T(□) | RCC(%)^{a} |
|---|---|---|---|---|---|
| 1 | 0.085 | 0.080 | 20 | 100 | 1 ± 1 |
| 2 | 0.042 | 0.080 | 20 | 120 | 20 ± 12 |
| 3 | 0.042 | 0.080 | 20 | 100 | 18 ± 8^{b} |
| 4 | 0.042 | 0.080 | 10 | 100 | 16 ± 7 |
| 5 | 0.021 | 0.080 | 20 | 120 | 20 ± 4 |
| 6 | 0.021 | 0.080 | 20 | 100 | 20 ± 10^{b} |
| 7 | 0.021 | 0.080 | 20 | 80 | 5 ± 2 |
| 8 | 0.021 | 0.10 | 20 | 100 | 8 ± 1 |
| 9 | 0.021 | 0.039 | 20 | 100 | 5 ± 4 |
| 10 | 0.021 | 0.059 | 20 | 100 | 19 ± 1 |
| 11 | 0.010 | 0.080 | 20 | 100 | 0 ± 0 |

General conditions: 300 μL of solvent.
^{a}Based-on n = 2 unless otherwise stated.
^{b}Based-on n = 4.

TABLE 3
Survery of solvents

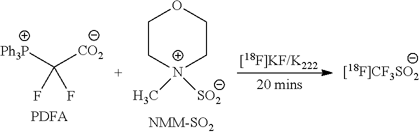

| | NMM-SO₂ | PDFA | | | |
|---|---|---|---|---|---|
| Entry | (mmol) | (mmol) | Solvent^{a} | T(□) | RCC(%)^{b} |
| 1 | 0.042 | 0.080 | DMA | 100 | 17 ± 5 |
| 2 | 0.021 | 0.080 | DMA | 100 | 20 ± 6 |
| 3 | 0.042 | 0.080 | DMI | 100 | 13 ± 3 |
| 4 | 0.021 | 0.080 | DMI | 100 | 20 ± 4 |
| 5 | 0.021 | 0.080 | Diglyme | 100 | 6 ± 3, |
| | | | | | 9 ± 2^{c} |
| 6 | 0.021 | 0.080 | 1,4-dioxane | 100 | 10 ± 5 |
| 7 | 0.021 | 0.080 | NMP | 100 | 12 ± 1 |
| 8 | 0.021 | 0.080 | Propylene | 100 | 25 ± 5 |
| | | | carbonate | | |
| 9 | 0.030 | 0.080 | Propylene | 100 | 22 ± 8 |
| | | | carbonate | | |
| 10^{d} | 0.030 | 0.080 | Propylene | 100 | 36 ± 2 |
| | | | carbonate | | |
| 11 | 0.030 | 0.080 | DMPU | 100 | 12 ± 2 |
| 12 | 0.021 | 0.080 | MeCN | 80 | 4 ± 1 |

General conditions: 300 μL of solvent.
^{a}Contained about 20-70 μL of DMF as [¹⁸F]KF/K₂₂₂ was dissolved in DMF and dispensed into reaction vials.
^{b}Based-on n = 2 except for entry 5.
^{c}Based-on n = 4.
^{d}PDFA is suspened in a solution of NMM-SO₂ in propylene carbonate and added to [¹⁸F]KF/K₂₂₂ in about 50 μL of DMF.

TABLE 4
Survery of phosphines and difluorocarbene source.

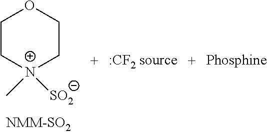

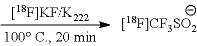

| Entry | Phosphine | :CF₂ source | Solvent | RCC(%)^{a} |
|---|---|---|---|---|
| 1 | PPh₃ | BrCF₂CO₂K | Propylene Carbonate | 1 ± 0 |
| 2 | PPh₃ | BrCF₂CO₂K | DMF | 3 ± 0 |
| 3^{b} | PPh₃ | BrCF₂CO₂K | DMA | 4 ± 1 |
| 4 | JohnPhos | BrCF₂CO₂K | DMA | 15 ± 3 |
| 5 | JohnPhos | ClCF₂CO₂CH₃ | DMF | 4 ± 0 |
| 6^{c} | JohnPhos | ClCF₂CO₂CH₃ | DMF | 10 ± 1 |
| 7 | JohnPhos | BrCF₂CO₂CH₃ | DMF | 18 ± 0 |
| 8^{c} | JohnPhos | BrCF₂CO₂CH₃ | DMF | 24 ± 1 |

General conditions: 0.03 mmol of NMM-SO₂, 0.08 mmol of :CF₂ source, 0.08 mmol of phosphine and 0.3 mL of solvent.
^{a}Based on n = 2.
^{b}0.03 mmol of phosphine
^{c}0.16 mmol of JohnPhos, 0.016 mmol of ClCF₂CO₂CH₃, and 0.06 mmol of NMM-SO₂ instead.

Mini Isolation Mode Screening

[¹⁸F]Fluoride (200-900 MBq) was separated from ¹⁸O-enriched-water using a Waters Sep-Pak light Accell Plus QMA cartridge (46 mg, activated with 2 mL water) and was subsequently released using a solution of Kryptofix-2.2.2 (15 mg) and K₂CO₃ (3 mg) in 1000 μL of MeCN/H2O, 4:1 into a 5 mL V-vial containing a magnetic stirrer bar in a concentrator.

The solution was dried with five cycles of azeotropic drying with anhydrous MeCN (5×200 μL) under a flow of N₂ at 105° C.

For the transfer of reagents to [¹⁸F]fluoride (See Table 5 entry 4 to 6, Table 6 entry 2 to 5): anhydrous solvent (300 μL) was added to a 1.5 mL vial containing amine-SO₂ adduct and difluorocarbene reagent before shaking to a fine suspension using a vortex mixer (˜10 seconds). The mixture was added to the dried [¹⁸F]KF/K₂₂₂ residue in a V-vial via syringe.

For the transfer of [¹⁸F]fluoride to reagents (See Table S5 entry 1 to 3, Table S6 entry 1 and Table S9): anhydrous solvent (300 μL) was added to the dried [¹⁸F]KF/K₂₂₂ residue and the mixture was heated at 100° C. for 2 min. The mixture was added to a V-vial containing amine-SO₂ adduct and difluorocarbene reagent.

After stirring at the specified temperature and time, the reaction mixture was cooled, taken up in 2% formic acid in water (6 mL) and rinsed over a WAX cartridge (activated with 3 mL water and 3 mL 2% formic acid in water). The vial was then rinsed with 2 mL of EtOH before eluting over the WAX cartridge. The cartridge was then rinsed with EtOH (4 mL) before eluting [¹⁸F]CF₃SO₂NH₄ into a 5 mL vial with 1% NH₃ in EtOH (3 mL) and a stream of N₂. An aliquot was removed to determine radiochemical purity by radioHPLC with an analytical Synergi 4 μm Hydro-RP 80 A column, 150×4.6 mm at a flow rate 1 mL/min. Activity was measured using a plastic scintillator/PMT detector. Radiochemical yields are calculated from activity and radiochemical purity:

Radioc [symbol] emical Yield (%)=Radioc [symbol] emical Purity (%)×Final Activity (MBq)/Initial Activity (MBq)

TABLE 5
Batch scale screening in DMF with variations in temperature
and concentration

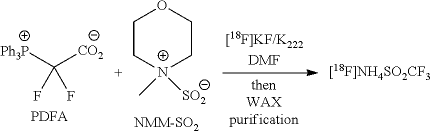

| | Initial | | Activity | | |
|---|---|---|---|---|---|
| | actitivty | Transfer | isolated | Radiochemical | |
| Entry | (MBq) | Efficiency(%)^{a} | from WAX | Purity (%) | RCY(%) |
| l^{b} | 902 | 49 | 32.5 | 72 | 4 |
| 2^{b} | 276 | 55 | 22.8 | 57 | 5 |
| 3^{c} | 489 | 28 | 35 | 61 | 4 |
| 4^{c} | 554 | N/A | 67 | 45 | 5 |
| 5^{c,d} | 445 | N/A | 105 | 54 | 13 |
| 6^{c,d,e} | 522 | N/A | 102 | 30 | 6 |

General conditions: 0.08 mmol of PDFA and 0.3 mL of DMF.
^{a}Transfer efficiency refers to the amount of [¹⁸F]KF/K₂₂₂ that was transferred to the reaction vial by dissolving in DMF after azeotropic drying.
^{b}0.2 mmol of NMM-SO₂, 10 min and 100 [symbol] .
^{c}0.3 mmol of NMM-SO₂, 20 min and 110 [symbol]
^{d}0.16 mmol of PDFA and 0.06 mmol of NMM-SO₂ were used.
^{e}fluoride was eluted with K₂₂₂ only. QMA was preconditioned with Na₂SO₄ according to Mossine et al.⁸

TABLE 6
Batch scale screening with propylene carbonate as the solvent

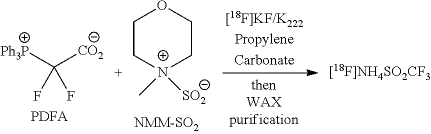

| | Initial | Transfer | Activity isolated from | Radiochemical | RCY |
|---|---|---|---|---|---|
| Entry | actitivty(MBq) | Efficiency (%)^{a} | WAX (MBq) | Purity (%) | (%) |
| 1^{b} | 231 | 70 | 10 | 0 | 0 |
| 2^{b} | 7700 | N/A | 850 | 0 | 0 |
| 3^{c,d,e} | 775 | N/A | 74 | 22 | 2 |
| 4^{c,d,f} | 894 | N/A | N/A | 0 | 0 |
| 5^{g} | 567 | N/A | 128 | 70 | 16 |

General conditions: 0.08 mmol of PDFA and 0.3 mL of DMF.
^{a}Transfer efficiency refers to the amount of [¹⁸F]KF/K₂₂₂ that was transferred to the reaction vial by dissolving in propylene carbonate after azeotropic drying.
^{b}0.2 mmol of NMM-SO₂, 10 min and 100 [symbol] .
^{c}0.3 mmol of NMM-SO₂, 20 min and 110 [symbol]
^{d}PDFA was suspended in a solution of NMM-SO₂.
^{e}50 μL of DMF was added.
^{f}Solvent comprised of 150 μL of DMF and 150 μL of propylene carbonate.
^{g}0.16 mmol of PDFA and 0.06 mmol of NMM-SO₂.

TABLE 7
Batch scale evaluation of difluorocarbene sources

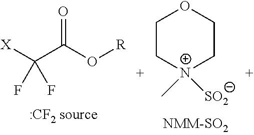

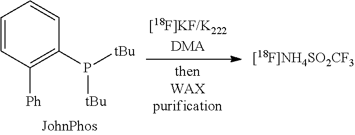

| | Initial actitivty | | Activity isolated from | Radiochemical | |
|---|---|---|---|---|---|
| Entry | (MBq) | :CF₂ source | WAX(MBq) | Purity (%) | RCY (%) |
| 1 | 427 | X = Br, R = K | 44 | 44 | 4 |
| 2 | 650 | X = Cl, R = CH₃ | 152 | 69 ± 1^{a} | 17 ± 1^{a} |
| 3^{b} | 520 | X = Cl, R = CH₃ | 113 | 63 | 14 |
| 4 | 793 | X = Br, R = CH₃ | 91 | 23 | 3 |

General conditions: 0.16 mmol of JohnPhos, 0.16 mmol of :CF₂ source, 0.06 mmol of NMM-SO₂, 300 μL of DMA, 110 [symbol] and 20 min of reaction time.
^{a}Based-on n = 2.
^{b}JohnPhos was added to the v-vial and the fluoride was dispensed into it, they were then azeotropically dried.
were then azeotropically dried.

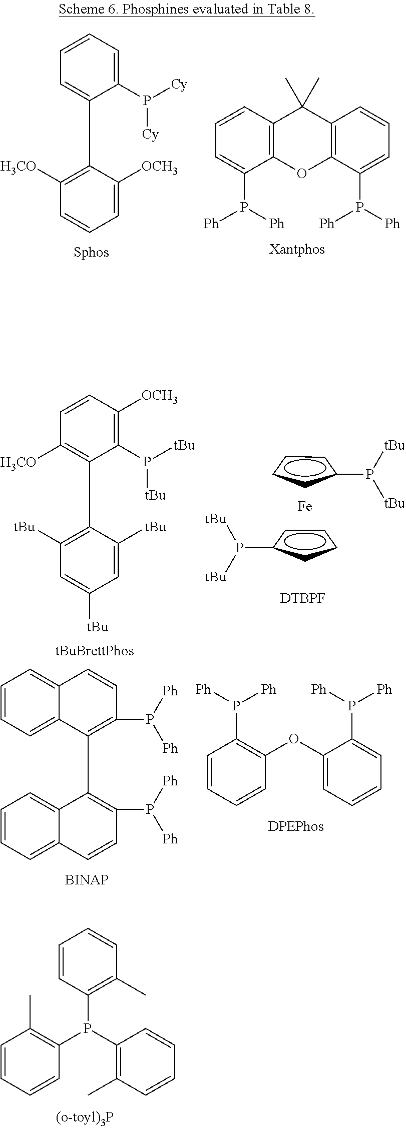

TABLE 8
Batch scale evaluation of phosphine

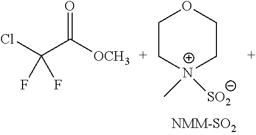

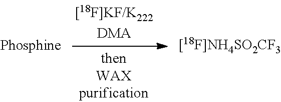

| | Initial | | Activity | | |
|---|---|---|---|---|---|
| | actitivty | | isolated from | Radiochemical | |
| Entry | (MBq) | Phosphine^{a,b} | WAX(MBq) | Purity (%) | RCY |
| 1 | 512 | SPhos | 99 | 67 | 13 |
| 2 | 368 | Xantphos | 45 | 56 | 7 |
| 3 | 259 | tBuBrettPhos | 54 | 68 | 14 |
| 4 | 324 | DTBPF | 23 | 65 | 5 |
| 5 | 479 | BINAP | 20 | 56 | 11 |
| 6 | 465 | DPEPhos | 72 | 60 | 9 |
| 7 | 376,407 | (o-tol)₃P | 121 | 60 ± 4 | 18 ± 3 |

General conditions: 0.16 mmol of Phosphine, 0.16 mmol of ClCF₂CO₂CH₃, 0.06 mmol of NMM-SO₂, 300 μL of DMA, 110 [symbol] and 20 min of reaction time.
^{a}Phosphine was added to the v-vial and the fluoride was dispensed into it, they were then azeotropically dried.
^{b}Refer to scheme 6 for structure of phosphine.

TABLE 9
Further variation with (o-toyl)₃P on batch scale

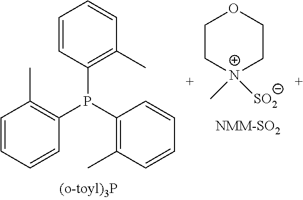

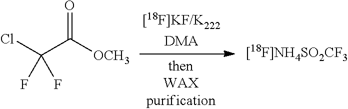

| | | | Activity | | |
|---|---|---|---|---|---|
| | Initial actitivty | Deviation from | isolated from | Radiochemical | |
| Entry | (MBq) | standard conditions^{a} | WAX(MBq) | Purity (%) | RCY (%) |
| 1 | 341 | N/A | 88 | 63 | 16 |
| 2 | 405 | [¹⁸F]CsF^{b} | 48 | 48 | 6 |
| 3 | 363 | 0.08 mmol of | 74 | 73 | 15 |
| | | phosphine | | | |
| 4 | 212 | Add 30 mL air to | 56 | 66 | 17 |
| | | reaction vial | | | |
| 5 | 399 | 10 min reaction | 116 | 72 | 21 |
| | | time | | | |

^{a}Standard conditions: 0.16 mmol of (o-toy1)₃P, 0.16 mmol of ClCF₂CO₂CH₃, 0.06 mmol of NMM-SO₂, 300 μL of DMA, 110 [symbol] and 20 min of reaction time. Phosphine was dissolved in DMA together with NMM-SO₂. Gentle heating with a hair dryer was used.
^{b}[¹⁸F] fluoride was eluted with a solution of dibenzo-24-crown-8 (8 mg), Cs₂C₂O₄ (2.1 mg) and Cs₂CO₃ (100 μL of 2.5 mg/mL solution), 100 μL of water and 800 μL of MeCN. 900 μL of this solution was used in the elution.

Isolation Procedure of [¹⁸F] Ammonium Trifluoromethanesulflnate

[¹⁸F]Fluoride (5-10 GBq) was separated from ¹⁸O-enriched-water using a Waters Sep-Pak light Accell Plus QMA cartridge (46 mg, activated with 2 mL H₂O) and was subsequently released using a solution of Kryptofix-2.2.2 (6.3 mg) and κ₂CO₃ (1 mg) in 1000 μL of MeCN/water, 4:1 into a 5 mL V-vial containing a magnetic stirrer bar in a concentrator. The solution was dried with five cycles of azeotropic drying with anhydrous MeCN (5×200 μL) under a flow of N₂ at 105° C. A suspension of PDFA (57 mg, 0.16 mmol), and N-methylmorpholine-SO₂ in Propylene Carbonate (300 μL) and DMF (50 μL) was then added and the reaction stirred at 125° C. (actual temperature is about 110° C.) for 20 minutes. After stirring at the specified temperature and time, the reaction mixture was cooled for 3 minutes, taken up in 2% formic acid in H₂O (6 mL) and added to a WAX cartridge (activated with 3 mL H₂O and 3 mL 2% formic acid in water). The vial was then rinsed with 2 mL of EtOH before eluting over the WAX cartridge. The WAX cartridge was then washed with EtOH (4 mL) before eluting [¹⁸F]CF₃SO₂NH₄ into a 5 mL vial with 1% NH₃ in EtOH (3 mL) and a stream of N₂. The ethanol was then removed under a stream of N₂ at 120° C. The vial was then cooled for 3 minutes before NH₄CO₂H (25 mM) was added, the vial shaken vigorously, and the solution loaded directly onto a 2 mL HPLC loop and injected onto a semi-Prep HPLC column (Synergi 4 μm Hydro-RP 250×10 mm) and eluted into a collection vial with 25 mM NH₄HCO₂ in water monitoring with UV (254 nm) and radioactive traces. The Molar Activity of [¹⁸F]NH₄SO₂CF₃ was assessed by radio-HPLC, using an analytical Synergi 4 μm Hydro-RP 80 Å column, 150×4.6 mm eluted with 1% MCCN/99% 25 mM NH₄HCO₂ in H₂O (isocratic 1 mL/min), monitoring with UV (220 nm) and radioactive traces.

Additional Notes for this Procedure:
Note that all EtOH must be evaporated from the 5 mL vial before loading onto HPLC.

It was found that even with minimal quantities of EtOH that the retention time of the product could drastically change and as such make purification challenging and less reliable.
It was found that [¹⁸F]CF₃SO₂NH₄ decomposes on WAX cartridges. To minimise decomposition, it is advised that trapping and elution be carried out as efficiently as possible (3 mL per minute).
To minimise the band width on HPLC, it is recommended that loading in no more than 1 mL of 25 mM NH₄CO₂H. Larger volumes of NH₄CO₂H can lead to peak broadening and therefore make purification challenging and less reliable.

Analysis of 5-[¹⁸F] Ammonium Trifluoromethanesulfonate ([¹⁸F]1)

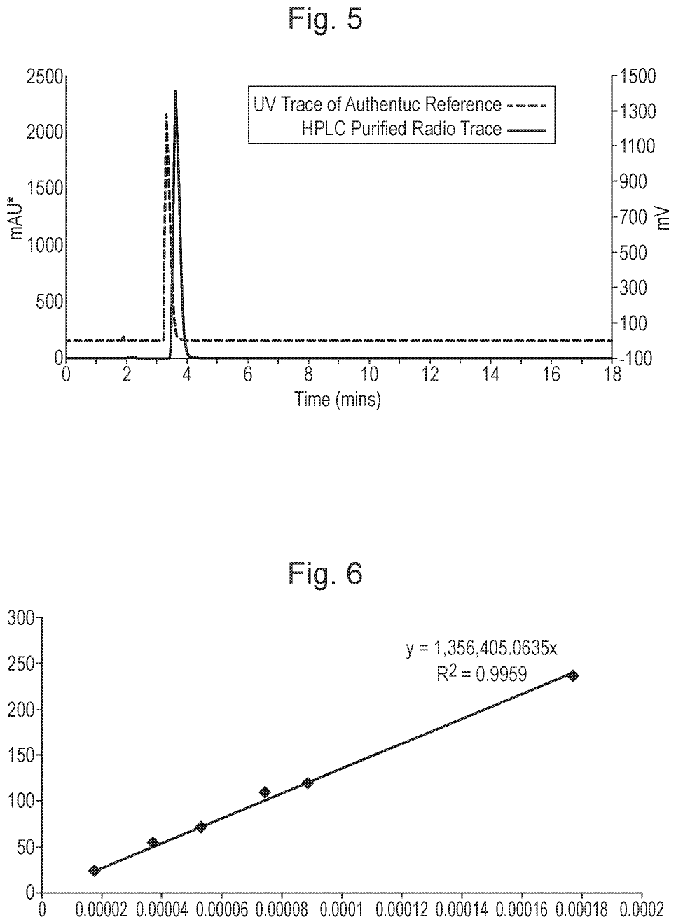

FIG. 5 shows the overlay of radiotrace for [¹⁸F]NH₄SO₂CF₃ and UV trace of authentic reference (220 nm). FIG. 6 shows the calibration curve for [¹⁸F]NH₄SO₂CF₃ molar activity determination.

TABLE 10
Determination of Molar Activity.

| | | Amount | | |
|---|---|---|---|---|
| | Area | [¹⁹F]SO₂CF₃ | | Molar Activity |
| Entry | (mAU × min)^{a} | (mmol) | Activity (MBq) | (GBq/μmol) |
| 1 | 59.49 | 4.01 × 10⁻⁵ | 0.6 | 0.01 |

General Procedure for the ¹⁸F-Trifluoromethylation of Peptides Using [¹⁸F]NH₄SO₂CF₃

An aliquot of a solution of [¹⁸F]NH₄SO₂CF₃ in 25 mM NH₄HCO₂H in water (20-300 μl, 10-100 MBq) was added to a v-vial which contained the substrate and the iron salt. A stock solution of 70% TBHP (aq) of the required concentration was prepared in the reaction solvent and an appropriate amount was taken up into a 1 mL syringe and added to the v-vial. The sealed vial was stirred at room temperature for 20 minutes.

Determination of Radiochemical Conversion: The reaction mixture was diluted with DMSO or 5% MeCN in water with 0.05% TFA and an aliquot was removed for analysis by and radio-HPLC to determine radiochemical yield. Radio-HPLC was performed on a Phenomenex Synergi Hydro RP 4 μm 80 Å 150×4.6 mm column, Agilent Zorbax 300 Extended C-18 4 μm 300 Å 150×4.6 mm column or Agilent Zorbax 300SB CN 4 μm 300 Å 150×4.6 mm column. Within the samples tested, all radioactive by-products are sufficiently soluble such that the radiochemical yield determined directly from HPLC and from isolation has no significant difference.

HPLC Eluent System for Peptides' Reactions

TABLE 11
Eluent A. Flow rate = 1.0 mL/min. Temp = 40° C.

| Time/min | MeCN^{a}(%) | Water^{a} (%) |
|---|---|---|
| 0.0 | 5 | 95 |
| 2.0 | 5 | 95 |
| 10.0 | 35 | 65 |
| 13.0 | 95 | 5 |
| 17.0 | 95 | 5 |
| 19.0 | 5 | 95 |
| 21.0 | 5 | 95 |

^{a}With 0.1% trifluoroacetic acid

TABLE 12
Eluent B. Flow rate = 1.0 mL/min. Temp = 40° C.

| Time/min | MeCN^{a}(%) | Water^{a} (%) |
|---|---|---|
| 0.0 | 5 | 95 |
| 2.0 | 5 | 95 |
| 15.0 | 45 | 55 |
| 17.5 | 95 | 5 |
| 20.0 | 95 | 5 |
| 22.0 | 5 | 95 |
| 24.0 | 5 | 95 |

^{a}With 0.1% trifluoroacetic acid

TABLE 13
Eluent C. Flow rate = 1.0 mL/min. Temp = 40° C.

| Time/min | MeCN^{a}(%) | Water^{a} (%) |
|---|---|---|
| 0.0 | 5 | 95 |
| 2.0 | 5 | 95 |
| 10.0 | 50 | 50 |
| 13.0 | 95 | 5 |
| 17.0 | 95 | 5 |
| 19.0 | 5 | 95 |
| 21.0 | 5 | 95 |

^{a}With 0.1% trifluoroacetic acid

TABLE 14
Eluent D. Flow rate = 1.0 mL/min. Temp = 40° C.

| Time/min | MeCN^{a}(%) | Water^{a} (%) |
|---|---|---|
| 0.0 | 15 | 85 |
| 2.0 | 15 | 85 |
| 17.5 | 45 | 55 |
| 20.0 | 95 | 5 |
| 22.0 | 15 | 85 |
| 24.0 | 15 | 85 |

^{a}With 0.1% trifluoroacetic acid

TABLE 15
Eluent E. Flow rate = 1.0 mL/min. Temp = 40° C.

| Time/min | MeCN^{a}(%) | Water^{a} (%) |
|---|---|---|
| 0.0 | 15 | 85 |
| 2.0 | 15 | 85 |
| 15.0 | 60 | 40 |
| 17.5 | 95 | 5 |
| 20.0 | 95 | 5 |
| 22.0 | 15 | 85 |
| 24.0 | 15 | 85 |

^{a}With 0.1% trifluoroacetic acid

TABLE 16
Eluent F. Flow rate = 1.0 mL/min. Temp = 40° C.

| Time/min | MeCN^{a}(%) | Water^{a} (%) |
|---|---|---|
| 0.0 | 15 | 85 |
| 2.0 | 15 | 85 |
| 15.0 | 60 | 40 |
| 17.5 | 95 | 5 |
| 20.0 | 95 | 5 |
| 22.0 | 15 | 85 |
| 24.0 | 15 | 85 |

^{a}With 0.1% trifluoroacetic acid

TABLE 17
Eluent G. Flow rate = 1.0 mL/min. Temp = 40° C.

| Time/min | MeCN^{a}(%) | Water^{a} (%) |
|---|---|---|
| 0.0 | 25 | 75 |
| 2.0 | 25 | 75 |
| 15.0 | 50 | 50 |
| 17.0 | 95 | 5 |
| 19.0 | 95 | 5 |
| 21.0 | 25 | 75 |
| 24.0 | 25 | 75 |

^{a}With 0.1% trifluoroacetic acid

TABLE 18
Eluent H. Flow rate = 1.0 mL/min. Temp = 40° C.

| Time/min | MeCN^{a}(%) | Water^{a} (%) |
|---|---|---|
| 0.0 | 5 | 95 |
| 2.0 | 5 | 95 |
| 15.0 | 27.5 | 72.5 |
| 17.0 | 95 | 5 |
| 19.0 | 5 | 95 |
| 21.0 | 5 | 95 |

^{a}With 0.1% trifluoroacetic acid

TABLE 19
Eluent I. Flow rate = 1.0 mL/min. Temp = 40° C.

| Time/min | MeCN^{a}(%) | Water^{a} (%) |
|---|---|---|
| 0.0 | 20 | 80 |
| 1.8 | 20 | 80 |
| 15.0 | 28 | 62 |
| 17.0 | 95 | 5 |
| 19.0 | 95 | 5 |
| 21.0 | 20 | 80 |
| 24.0 | 20 | 80 |

^{a}With 0.1% trifluoroacetic acid

TABLE 20
Eluent J. Flow rate =1.0 mL/min. Temp = 40° C.

| Time/min | MeCN^{a}(%) | Water^{a} (%) |
|---|---|---|
| 0.0 | 8 | 92 |
| 1.8 | 8 | 92 |
| 18.0 | 30 | 70 |
| 21.0 | 95 | 5 |
| 24.0 | 95 | 5 |
| 25.0 | 8 | 92 |
| 27.0 | 8 | 92 |

^{a}With 0.1% trifluoroacetic acid

Isolation of Radiochemially Pure Products

Semi prep HPLC of crude reactions were performed with Phenomenex Onyx Monolithic Semi-PREP C18 100×10 mm.

TABLE S21
Semi-Prep HPLC program. Temp = 40° C.

| Time/min | MeCN^{a} (%) | Water^{a} (%) | Flow rate (mL/min) |
|---|---|---|---|
| 0 | 5 | 95 | 4.0 |
| 1 | 5 | 95 | 10 |
| 2 | 5 | 95 | 10 |
| 15 | 35 | 65 | 10 |
| 16 | 80 | 20 | 10 |
| 18 | 5 | 95 | 6.0 |
| 20 | 5 | 95 | 4.0 |

^{a}With 0.1% trifluoroacetic acid

L-Tyr[ortho-CF₂¹⁸F]

### (S)-2-amino-3-(4-hydroxy-3-([¹⁸F]trifluoromethyl)phenyl)propanoic acid

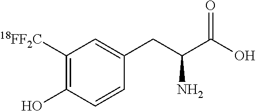

Prepared following the general procedure using L-tyrosine, Fe(NO₃)₃.9H₂O and 70% TBHP (aq). The amount and condition used are specified in Table 22. Radiochemical conversion was determined by HPLC (Eluent H, Table 18, at 40° C. with the Phenomenex Synergi Hydro RP 4 μm 80 Å 150×4.6 mm column). The HPLC overlay of crude radio-trace of L-tyrosine reaction with [¹⁸F]NH₄SO₂CF₃ and UV trace of authentic reference for both the ortho and meta-CF₃ products are provided in FIGS. 7 and 8.

TABLE 22
Details for optimization of L-Tyrosine.

| | | | | Amount of | Amount of | |
|---|---|---|---|---|---|---|
| | | | | [¹⁸F]NH₄SO₂CF₃ in | 10% AcOH in | |
| | Substrate | Fe(NO₃)₃^{a} | TBHP | 25 mM AF^{b} dispensed | 25 mM AF^{b} | T |
| Entry | (mmol) | (mmol) | (mmol) | (mL) | added (mF) | (° C.) |
| 1 | 0.12 | 0 | 0.12 | 0.125 | 0.125 | 60 |
| 2 | 0.12 | 0.06 | 0.00 | 0.050 | 0.25 | 60 |
| 3 | 0.12 | 0.06 | 0.12 | 0.100 | 0.20 | 60 |
| 4^{c} | 0.12 | 0.06 | 0.12 | 0.050 | 0.25 | 60 |
| 5 | 0.12 | 0.06 | 0.06 | 0.050 | 0.25 | 60 |
| 6 | 0.12 | 0.06 | 0.06 | 0.065 | 0.235 | 60 |
| 7 | 0.06 | 0.06 | 0.12 | 0.070 | 0.23 | 60 |
| 8 | 0.03 | 0.06 | 0.12 | 0.080 | 0.22 | 60 |
| 9 | 0.03 | 0.06 | 0.12 | 0.150 | 0.15 | 40 |

^{a}nonahydrate was used.
^{b}AF = Ammonium Formate.

TABLE 23
Results for Table 22 ortho-CF₃

| | | | | | | Sample |
|---|---|---|---|---|---|---|
| | RCC^{a} | | | | | Standard |
| Entry | 1 | 2 | 3 | 4 | Average | Deviation |
| 1 | 0 | 0 | — | — | 0 | 0 |
| 2 | 0 | 0 | — | — | 0 | 0 |
| 3 | 53.72 | 56.03 | — | — | 54.9 | 1.6 |
| 4 | 38.01 | 42.12 | — | — | 40.1 | 2.9 |
| 5 | 50.18 | 51.92 | — | — | 51.1 | 1.2 |
| 6 | 55.86 | 5507 | — | — | 55.5 | 0.6 |
| 7 | 56.45 | 61.81 | — | — | 59.1 | 3.8 |
| 8 | 50.64 | 48.70 | — | — | 49.7 | 1.4 |
| 9 | 19.79 | 20.94 | 18.78^{b} | 28.99 | 22.1 | 4.7 |

^{a}Determined from relative area of all radiopeaks in HPLC.
^{b}HPLC was performed with Zorbax 300 Extended C-18 4 μm 300 Å 150 × 4.6 mm column instead.

TABLE 24
Results for Table 22 meta-CF₃

| | | | | | | Sample |
|---|---|---|---|---|---|---|
| | RCC^{a} | | | | | Standard |
| Entry | 1 | 2 | 3 | 4 | Average | Deviation |
| 1 | 0 | 0 | — | — | 0.0 | 0.0 |
| 2 | 0 | 0 | — | — | 0.0 | 0.0 |
| 3 | 9.00 | 11.57 | — | — | 10.3 | 1.8 |
| 4 | 7.73 | 6.58 | — | — | 7.2 | 0.8 |
| 5 | 8.88 | 11.50 | — | — | 10.2 | 1.9 |
| 6 | 12.54 | 11.85 | — | — | 12.2 | 0.5 |
| 7 | 12.84 | 14.53 | — | — | 13.7 | 1.2 |
| 8 | 10.93 | 10.93 | — | — | 10.9 | 0.0 |
| 9 | 3.71 | 4.72 | 2.85^{b} | 5.10 | 4.1 | 1.0 |

L-Trp[W-2-CF₂¹⁸F]

### (S)-1-carboxy-2-(2-(trifluoromethyl)-1H-indol-3-yl)ethan-1-aminium trifluoroacetate

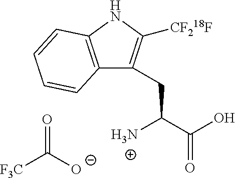

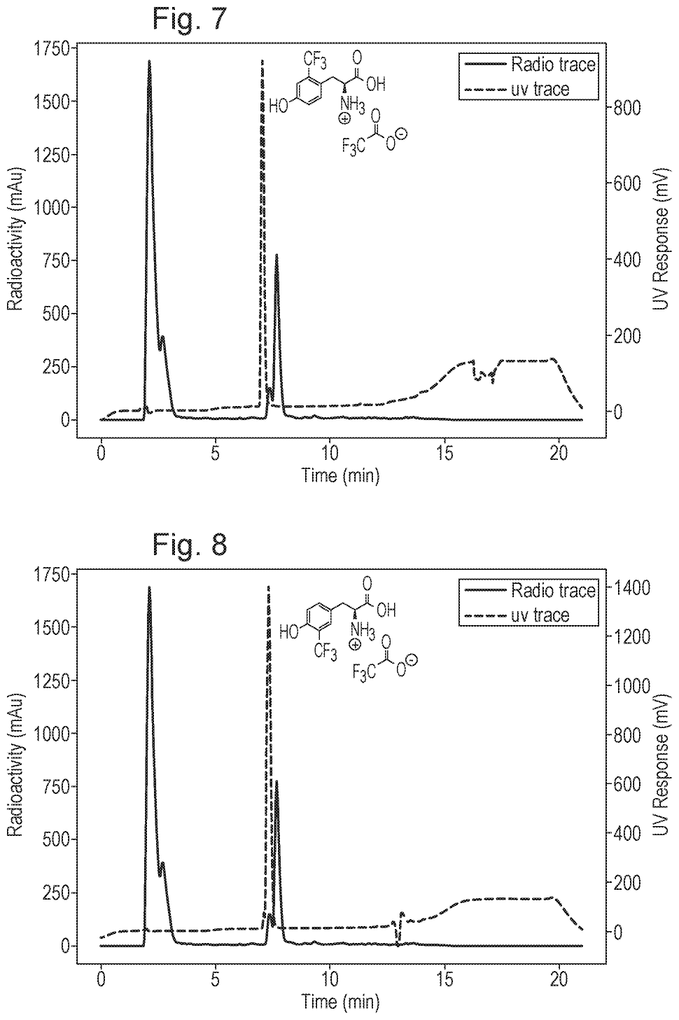

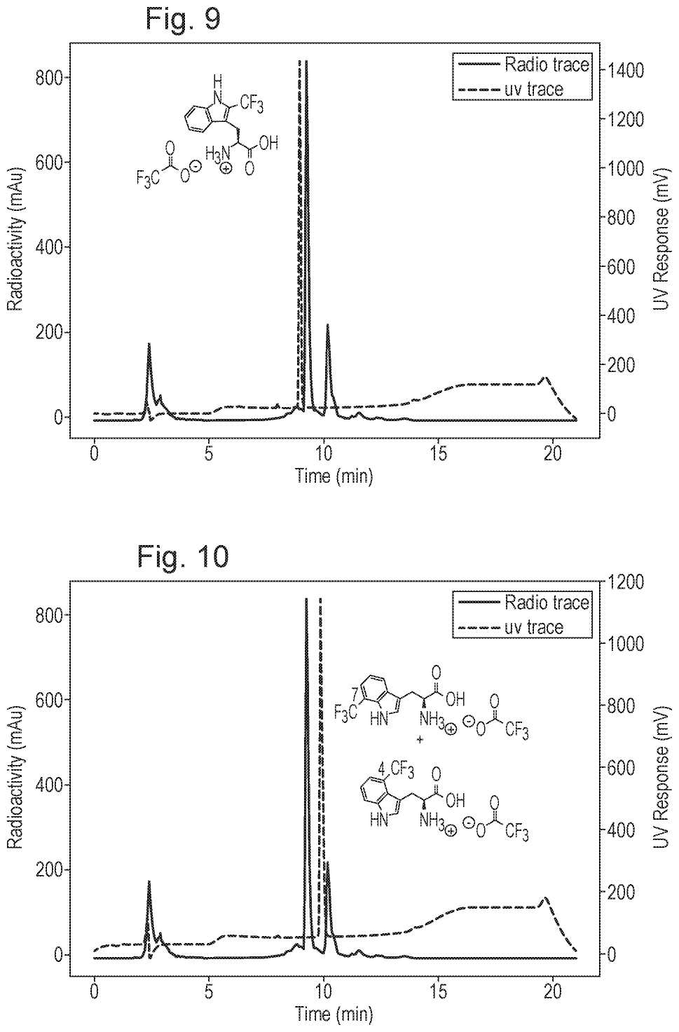

Prepared following the general procedure using L-tryptophan (0.03 mmol, 5.4 mg), FeCl₃ (0.06 mmol, 6.1 mg) and 70% TBHP in water (0.12 mmol, 16.7 μL), the amount of solvent is specified in Table 25. Radiochemical conversion was determined by radio-HPLC (Refer to Table 26 and Table 27). HPLC overlay spectra for the products are provided in FIGS. 9 and 10. A small amount of di-trifluoromethylation on Trp is assumed to be present in FIG. 10.

TABLE 25
Amount of solvent used for each reaction.

| | Amount of | |
|---|---|---|
| | [¹⁸F]NH₄SO₂CF₃ in | |
| | 25 mM | Amount of |
| Entry | NH₄HCO₂ dispensed/mL | DMSO added/mL |
| 1 | 0.100 | 0.20 |
| 2 | 0.080 | 0.22 |
| 3 | 0.125 | 0.125 |
| 4 | 0.100 | 0.150 |
| 5 | 0.150 | 0.150 |
| 6 | 0.075 | 0.225 |

TABLE 26
HPLC details and RCC for L-Trp[2-CF₃]

| Reaction | RCC^{a} (%) | HPLC column | Eluent |
|---|---|---|---|
| 1 | 48.22 | Synergi | C^{b} |
| 2 | 36.45 | Zorbax 300 Extended C-18 | B^{c} |
| 3 | 12.47 | Synergi | C^{b} |
| 4 | 22.28 | Synergi | C^{b} |
| 5 | 16.68 | Zorbax 300 Extended C-18 | B^{c} |
| 6 | 24.52 | Zorbax 300 Extended C-18 | B^{c} |
| Average | 26.77 | | |
| Sample Standard | 13.31 | | |
| Deviation | | | |

^{a}Determined from relative area of all radiopeaks in HPLC.
^{b}Refer to Table 13.
^{c}Refer to Table 12.

TABLE 27
HPLC details and RCC for L-Trp[4-CF₃] and L-Trp[7-CF₃].

| Reaction | RCC^{a} (%) | HPLC column | Eluent |
|---|---|---|---|
| 1 | 15.22 | Synergi | C^{b} |
| 2 | 10.72 | Zorbax 300 Extended C-18 | B^{c} |
| 3 | 5.37 | Synergi | C^{b} |
| 4 | 9.59 | Synergi | C^{b} |
| 5 | 7.04 | Zorbax 300 Extended C-18 | B^{c} |
| 6 | 10.09 | Zorbax 300 Extended C-18 | B^{c} |
| Average | 9.67 | | |
| Sample Standard | 3.39 | | |
| Deviation | | | |

^{a}Determined from relative area of all radiopeaks in HPLC.
^{b}Refer to Table 13.
^{c}Refer to Table 12.

Isolation with an OASIS HLB plus: The HLB plus cartridge is activated with 6 mL of EtOH, blown dry then washed with 6 mL of H₂O. The crude reaction mixture (4.96 MBq) was diluted with 4 mL of H₂O and taken up into a syringe. The crude mixture was eluted slowy through the HLB plus cartridge. The vial with the crude reaction mixture was washed with 1 mL of H₂O and the washing was eluted through the same HLB plus. 4 mL of H₂O was eluted through the HLB plus cartridge. 2 mL of EtOH was used to elute the product from the HLB cartridge. The eluted activity was measured to be 2.12 MBq. HPLC analysis was performed on the eluted mixture, the radiochemical purity of the product is 73.67%. The decay uncorrected radiochemical yield is 31.5%.

TyrTrp[W-2-CF₂¹⁸F]

### (S)-1-(((S)-1-carboxy-2-(2-([¹⁸F]trifluoromethyl)-1H-indol-3-yl)ethyl)amino)-3-(4-hydroxyphenyl)-1-oxopropan-2-aminium trifluoroacetate

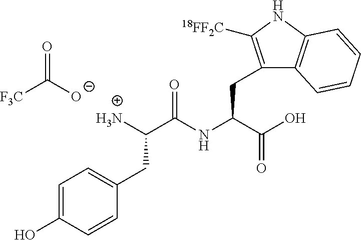

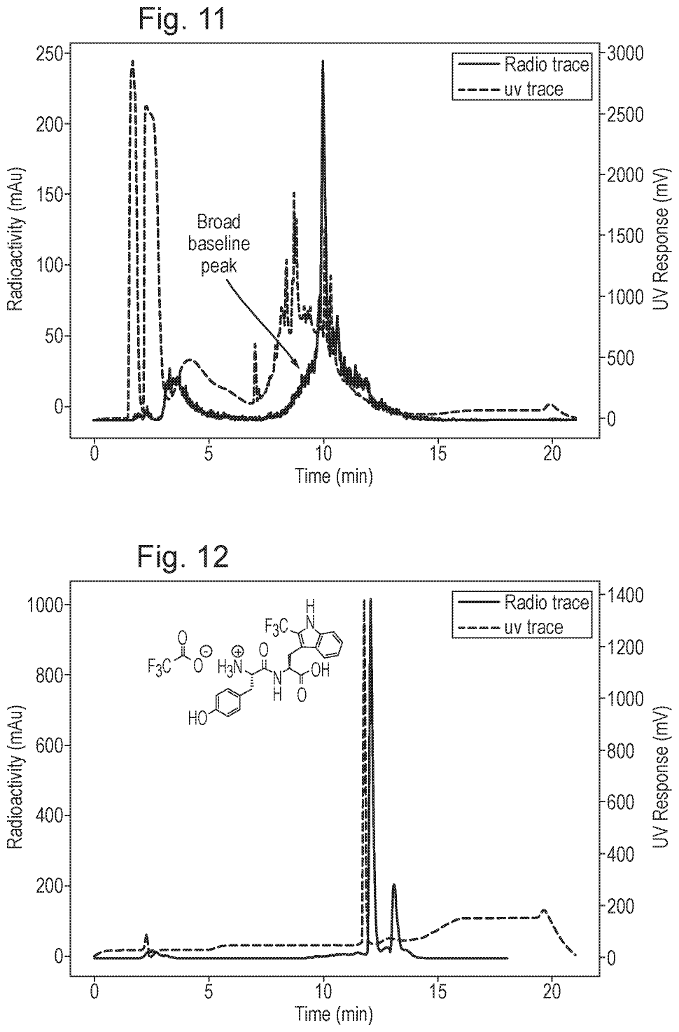

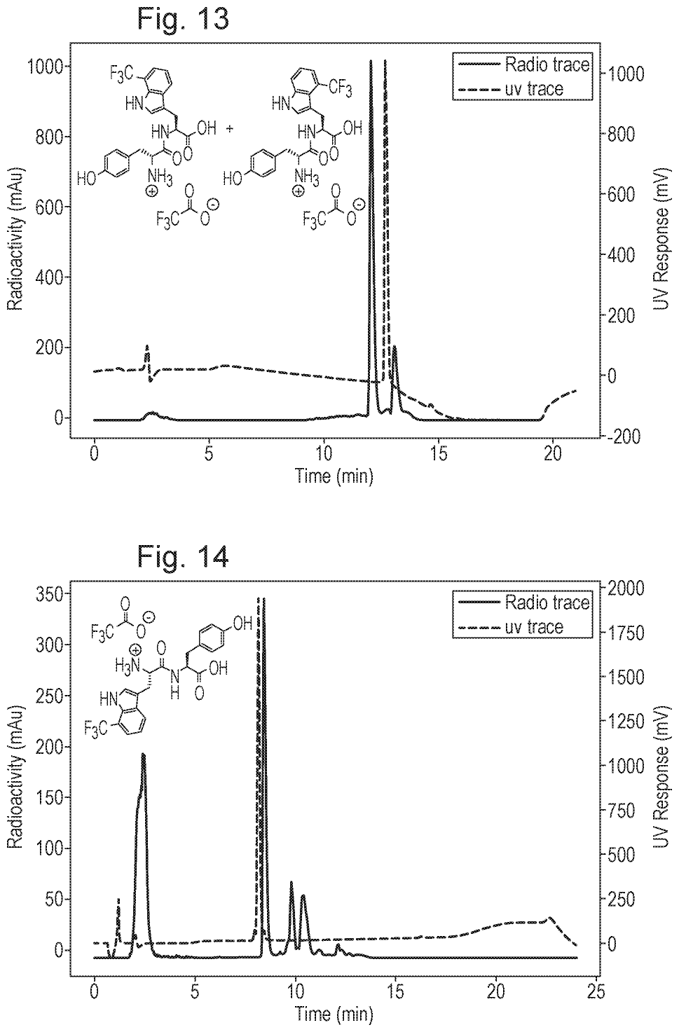

Prepared following the general procedure using H-Tyr-Trp-OH (0.015 mmol), Fe(NO₃)₃.9H₂O (0.03 mmol) and 70% TBHP (0.06 mmol), except in the case of Table S28 Entry 1 where substrates and reagents are double and Entry 6 where FeCl₃ was used instead of Fe(NO₃)₃□9H₂O. The reaction mixture was stirred at 40° C. for 20 min. Radiochemical conversion was determined by radio-HPLC (Eluent A, Table S11, at 40° C. with Phenomenex Synergi Hydro RP 4 μm 80 Å 150×4.6 mm column unless otherwise indicated). HPLC overlay spectra for the crude reaction for Table 38, entry 2, and for the separated reaction products are provided in FIGS. 11, 12 and 13.

TABLE 28
Experimental details for condition screening of H-Tyr-Trp-OH.

| | | | Amount of | | |
|---|---|---|---|---|---|
| | Solvent added in addition to | | [¹⁸F]NH₄SO₂CF₃ in | | |
| | the one dispensed | | 25 mM | | |
| | | Amount | AF dispensed | | |
| Entry | Type | (mL) | (mL) | Total Vol./mL | Conc/[M] |
| 1 | 10% AcOH in | 0.15 | 0.100 | 0.15 | 0.10 |
| | 25 mM AF(aq) | | | | |
| 2 | 25 mM AF(aq) | 0.050 | 0.100 | 0.15 | 0.10 |
| 3 | 1M pH 6 | 0.050 | 0.10 | 0.15 | 0.10 |
| | NaOAc/NH₃ | | | | |
| | Buffer | | | | |
| 4^{c} | pH 10 | 0.10 | 0.050 | 0.15 | 0.10 |
| 5 | DMSO | 0.10 | 0.050 | 0.15 | 0.10 |
| 6 | DMSO(FeCl₃) | 0.050 | 0075 | 0.125 | 0.12 |
| 7 | DMSO | 0.30 | 0.050 | 0.35 | 0.043 |
| 8 | DMSO | 0.60 | 0.100 | 0.7 | 0.021 |

TABLE 29
Results for Table 28 - H-Tyr-Trp(2-CF₃)-OH.

| | | | | | | Sample |
|---|---|---|---|---|---|---|
| | RCC^{a} | | | | | Standard |
| Entry | 1 | 2 | 3 | 4 | Average | Deviation |
| 1 | 39.80 | 46.35 | — | — | 43.1 | 4.6 |
| 2 | 27.06 | 21.58 | — | — | 24.3 | 3.9 |
| 3 | 34.2 | 38.8 | — | — | 36.5 | 3.3 |
| 4 | 27.14 | 17.19 | — | — | 22.2 | 7.0 |
| 5 | 43.59 | 39.63 | 43.00 | 52.94 | 44.8 | 5.7 |
| 6 | 68.76 | 58.50 | 58.02 | — | 61.8 | 6.1 |
| 7 | 39.90 | 41.95 | — | — | 40.9 | 1.4 |
| 8 | 24.67 | 13.49^{b} | — | — | 19.1 | 7.9 |

^{a}Determined from relative area of all radiopeaks in HPLC.
^{b}HPLC was performed with Zorbax 300 Extended C-18 4 μm 300 Å 150 × 4.6 mm column instead.
c broad and extended radio-peak observed see Figure.

TABLE 30
Results for Table 28 - H-Tyr-Trp(4-
CF₃)-OH + H-Tyr-Trp(7-CF₃)-OH

| | | | | | | Sample |
|---|---|---|---|---|---|---|
| | RCC^{a} | | | | | Standard |
| Entry | 1 | 2 | 3 | 4 | Average | Deviation |
| 5 | 14.69 | 15.36 | 13.53 | 16.44 | 15.0 | 1.2 |
| 6 | 19.14 | 19.80 | 20.11 | — | 19.7 | 0.5 |

^{a}Determined from relative area of all radiopeaks in HPLC.

Isolation with an OASIS HLB plus: The HLB plus cartridge is activated with 6 mL of MeOH, blown dry then washed with 6 mL of H₂O. The crude reaction mixture (5.86 MBq) was diluted with 4 mL of H₂O and taken up into a syringe. The crude mixture was eluted slowly through the HLB plus cartridge. The vial with the crude reaction mixture was washed with 1 mL of H₂O and the washing was eluted through the same HLB plus. 4 mL of H₂O was eluted through the HLB plus cartridge. 2 mL of EtOH was used to elute the product from the HLB cartridge. The eluted activity was measured to be 4.4 MBq. HPLC analysis was performed on the eluted mixture, the radiochemical purity of the product is 53.95%. The decay uncorrected radiochemical yield (RCY) is 27.9%. The RCY determined directly from HPLC without isolation of the crude reaction mixture is 28.3%.

Trp[W-2-CF₂¹⁸F]Tyr

### (S)-1-(((S)-1-carboxy-2-(4-hydroxyphenyl)ethyl)amino)-1-oxo-3-(2-([¹⁸F]trifluoromethyl)-1H-indol-3-yl)propan-2-aminium 2,2,2-trifluoroacetate

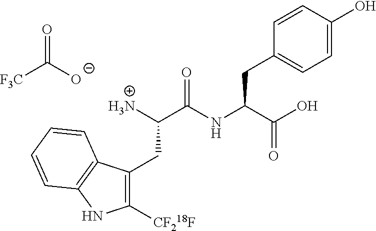

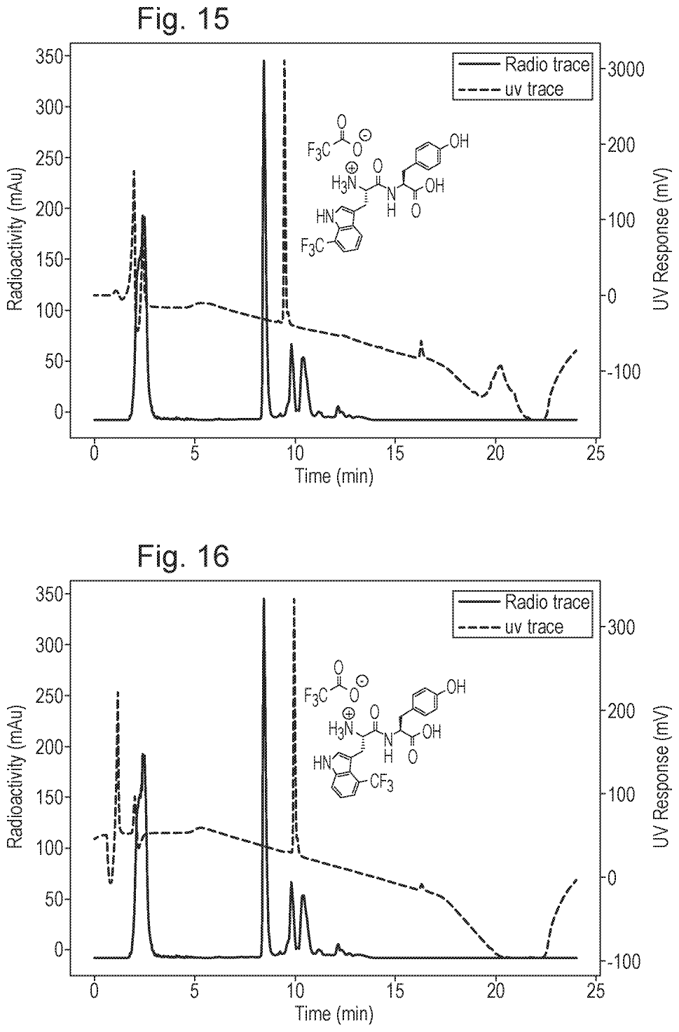

Prepared following the general procedure using H-Trp-Tyr-OH (0.015 mmol, 5.5 mg), Fe(NO₃)₃.9H₂O (0.03 mmol, 12.1 mg) and 70% TBHP in water (0.06 mmol, 8.2 μL), the amount of solvent is specified in Table 31. Radiochemical conversion was determined by radio-HPLC (Refer to Table 32, Table 33 and Table 34). HPLC overlay spectra for the products are provided in FIGS. 14, 15 and 16.

TABLE 31
Amount of solvent used for each reaction.

| | Amount of [¹⁸F] | Amount of |
|---|---|---|
| | NH₄SO₂CF₃ in 25 mM | DMSO |
| | NH₄HCO₂ dispensed/mL | added/mL |
| 1 | 0.150 | 0 |
| 2 | 0.150 | 0 |
| 3 | 0.075 | 0.075 |
| 4 | 0.075 | 0.075 |

TABLE 32
HPLC details and RCC for H-Trp[2-CF₃]-Tyr-OH

| Reaction | RCC^{a} (%) | HPLC column | Eluent |
|---|---|---|---|
| 1 | 19.43 | Synergi | A^{b} |
| 2 | 19.98 | Zorbax 300 Extended C-18 | A^{b} |
| 3 | 27.61 | Synergi | F^{c} |
| 4 | 22.24 | Zorbax 300 Extended C-18 | A^{b} |
| Average | 22.3 | | |
| Sample Standard | 3.7 | | |
| Deviation | | | |

^{a}Determined from relative area of all radiopeaks in HPLC.
^{b}Table 11.
^{c}Refer to Table 16.

TABLE 33
HPLC details and RCC for H-Trp[7-CF₃]-Tyr-OH

| Reaction | RCC^{a} (%) | HPLC column | Eluent |
|---|---|---|---|
| 1 | 4.57 | Synergi | A^{b} |
| 2 | 4.93 | Zorbax 300 Extended C-18 | A^{b} |
| 3 | 8.02 | Synergi | F^{c} |
| 4 | 7.03 | Zorbax 300 Extended C-18 | A^{b} |
| Average | 6.1 | | |
| Sample Standard | 1.7 | | |
| Deviation | | | |

^{a}Determined from relative area of all radiopeaks in HPLC.
^{b}Table 11.
^{c}Refer to Table 16.

TABLE 34
HPLC details and RCC for H-Trp[4-CF₃]-Tyr-OH

| Reaction | RCC^{a} (%) | HPLC column | Eluent |
|---|---|---|---|
| 1 | 6.68 | Synergi | A^{b} |
| 2 | 7.04 | Zorbax 300 Extended C-18 | A^{b} |
| 3 | 10.44 | Synergi | F^{c} |
| 4 | 10.54 | Zorbax 300 Extended C-18 | A^{b} |
| Average | 8.7 | | |
| Sample Standard | 2.1 | | |
| Deviation | | | |

^{a}Determined from relative area of all radiopeaks in HPLC.
^{b}Table 11.
^{c}Refer to Table 16.

PheTyr[ortho-CF₂¹⁸F]

### (S)-1-(((S)-1-carboxy-2-(4-hydroxy-3-([¹⁸F]trifluoromethyl)phenyl)ethyl)amino)-1-oxo-3-phenylpropan-2-aminium trifluoroacetate

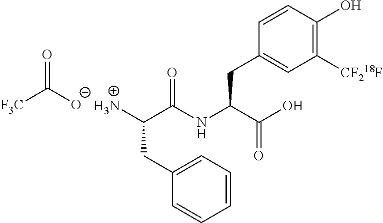

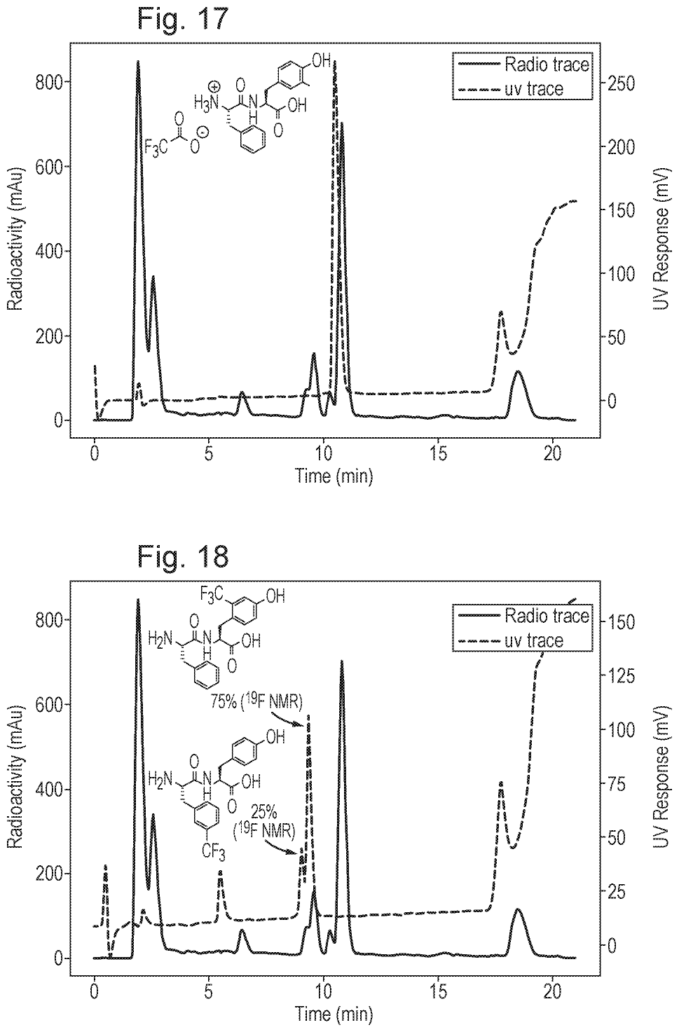

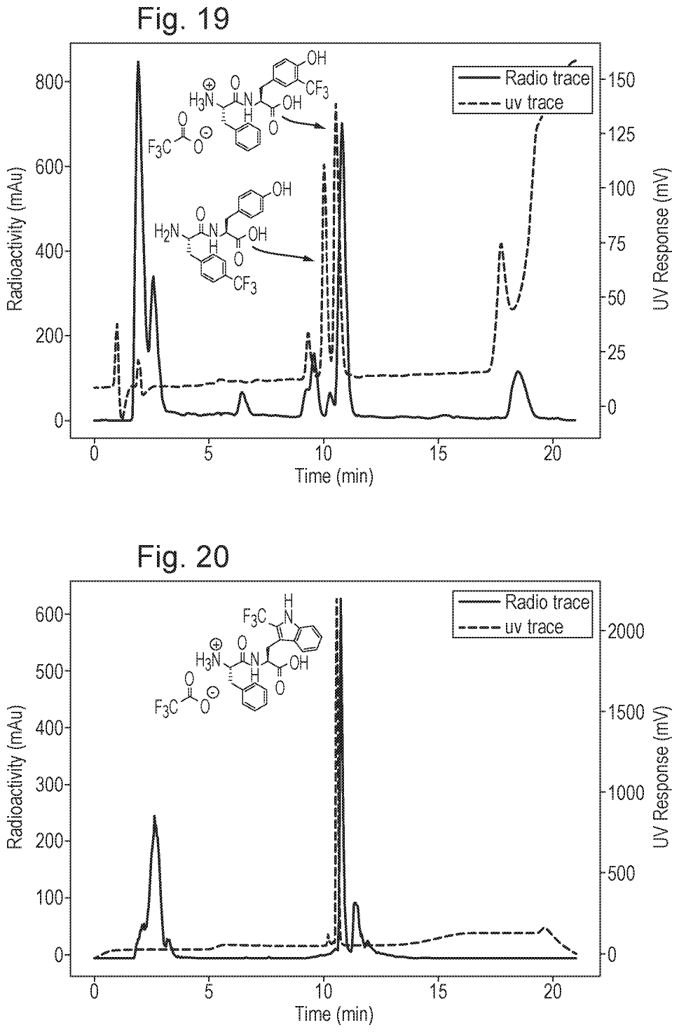

Prepared following the general procedure using H-Phe-Try-OH (0.03 mmol, 9.9 mg), Fe(NO₃)₃□9H₂O (0.06 mmol, 24.2 mg) and 70% TBHP in water (0.12 mmol, 16.7 μL), the amount of solvent is specified in Table 35. Radiochemical conversion was determined by radio-HPLC (Refer to Table 36 and Table 37). HPLC overlay spectra for the products are provided in FIGS. 17, 18 and 19.

TABLE 35
Amount of solvent used for each reaction.

| | Amount of | |
|---|---|---|
| | [¹⁸F]NH₄SO₂CF₃ in | Amount of |
| | 25 mM | 10% AcOH in |
| | NH₄HCO₂ | 25 mM AF added |
| | dispensed/mL | (mL) |
| 1 | 0.150 | 0.15 |
| 2 | 0.150 | 0.15 |
| 3 | 0.130 | 0.20 |
| 4 | 0.130 | 0.20 |
| 5 | 0.100 | 0.20 |
| 6 | 0.050 | 0.25 |

TABLE 36
HPLC details and RCC for H-Phe-Tyr[ortho-CF₃]-OH

| Reaction | RCC^{a} (%) | HPLC column | Eluent |
|---|---|---|---|
| 1 | 30.69 | Synergi | A^{b} |
| 2 | 29.30 | Synergi | A^{b} |
| 3 | 36.58 | Synergi | C^{c} |
| 4 | 38.60 | Synergi | C^{c} |
| 5 | 47.41 | Synergi | E^{d} |
| 6 | 30.52 | Synergi | I^{e} |
| Average | 36.5 | | |
| Sample Standard | 7.2 | | |
| Deviation | | | |

^{a}Determined from relative area of all radiopeaks in HPLC.
^{b}Refer to Table 11
^{c}Refer to Table 13.
^{d}Refer to Table 14.^{e}Refer to Table 19.

TABLE 37
HPLC details and RCC for H-Phe-Tyr[meta-CF₃]-0H + H-Phe[ m-
+ p- + CF₃] + unknown

| Reaction | RCC^{a} (%) | HPLC column | Eluent |
|---|---|---|---|
| 1 | 7.50 | Synergi | A^{b} |
| 2 | 7.63 | Synergi | A^{b} |
| 3 | 8.02 | Synergi | C^{c} |
| 4 | 7.23 | Synergi | C^{c} |
| 5 | 11.62 | Synergi | E^{d} |
| 6 | 10.12 | Synergi | I^{e} |
| Average | 8.47 | | |
| Sample Standard | 1.8 | | |
| Deviation | | | |

^{a}Determined from relative area of all radiopeaks in HPLC.
^{b}Refer to Table 11.
^{c}Refer to Table 13.
^{d}Refer to Table 14.
^{e}Refer to Table 19.

PheTrp[W-2-CF₂¹⁸F]

### (S)-1-(((S)-1-carboxy-2-(2-([¹⁸F]trifluoromethyl)-1H-indol-3-yl)ethyl)amino)-1-oxo-3-phenylpropan-2-aminium 2,2,2-trifluoroacetate

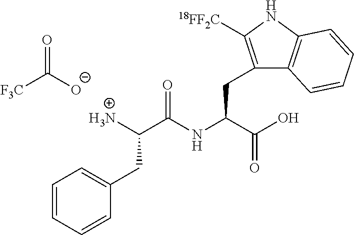

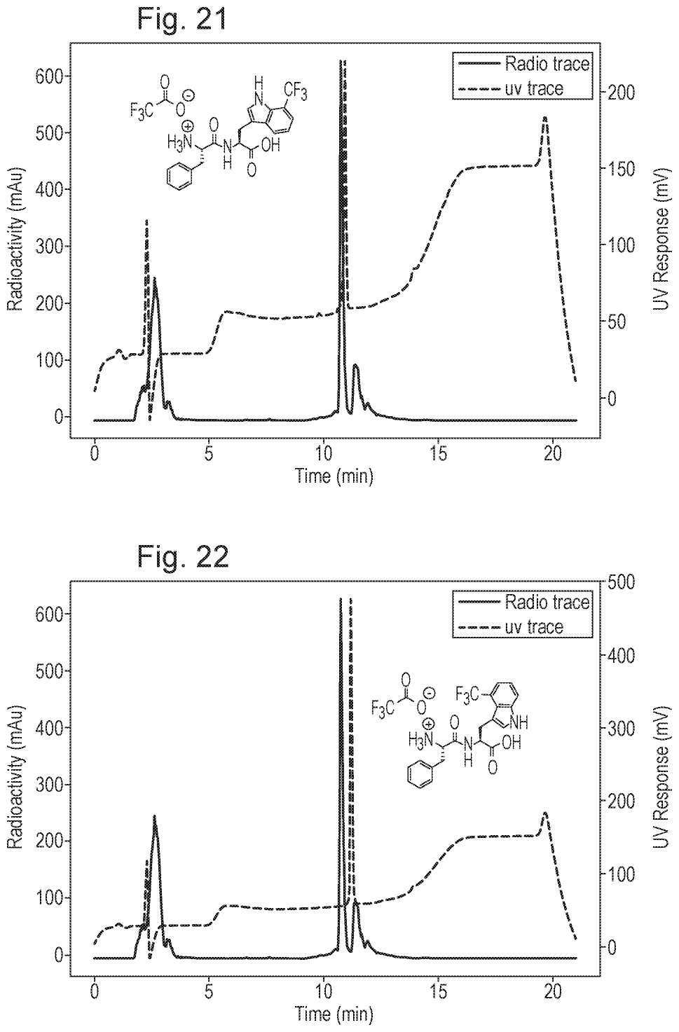

Prepared following the general procedure using H-Phe-Trp-OH (0.015 mmol, 5.3 mg), Fe(NO₃)₃.9H₂O (0.03 mmol, 12.1 mg) and 70% TBHP in water (0.06 mmol, 8.2 μL), the amount of solvent is specified in Table 38. Radiochemical conversion was determined by radio-HPLC (Refer to Table 39 and Table 40). HPLC overlay spectra for the products are provided in FIGS. 20, 21 and 22.

TABLE 38
Amount of solvent used for each reaction.

| | Amount of | |
|---|---|---|
| | [¹⁸F]NH₄SO₂CF₃ in | |
| | 25 mM | |
| | NH₄HCO₂ | |
| | dispensed/mL | Amount of DMSO added/mL |
| 1 | 0.100 | 0.050 |
| 2 | 0.100 | 0.050 |
| 3 | 0.030 | 0.120 |

TABLE 39
HPLC details and RCC for H-Phe-Trp[2-CF₃]-OH

| Reaction | RCC^{a} (%) | HPLC column | Eluent |
|---|---|---|---|
| 1 | 50.89 | Synergi | A^{b} |
| 2 | 53.86 | Zorbax 300 Extended C-18 | A^{b} |
| 3 | 33.60 | Synergi | C^{c} |
| Average | 46.1 | | |
| Sample Standard | 10.9 | | |
| Deviation | | | |

^{a}Determined from relative area of all radiopeaks in HPLC.
^{b}Refer to Table S11.
^{c}Refer to Table S13.

TABLE 40
HPLC details and RCC for H-Phe-Trp[
4-CF₃]-OH and H-Phe-Trp[7-CF₃]-OH

| Reaction | RCC^{a} (%) | HPLC column | Eluent |
|---|---|---|---|
| 1 | 29.38 | Synergi | A^{b} |
| 2 | 17.00 | Zorbax 300 Extended C-18 | A^{b} |
| 3 | 12.07 | Synergi | C^{c} |
| Average | 19.5 | | |
| Sample Standard | 8.9 | | |
| Deviation | | | |

^{a}Determined from relative area of all radiopeaks in HPLC.
^{b}Refer to Table S11.
^{c}Refer to Table S13.

MetTrp[W-2-CF₂¹⁸F]

### (S)-1-(((S)-1-carboxy-2-(2-([¹⁸F]trifluoromethyl)-1H-indol-3-yl)ethyl)amino)-4-(methylthio)-1-oxobutan-2-aminium trifluoroacetate

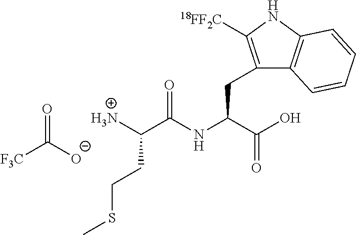

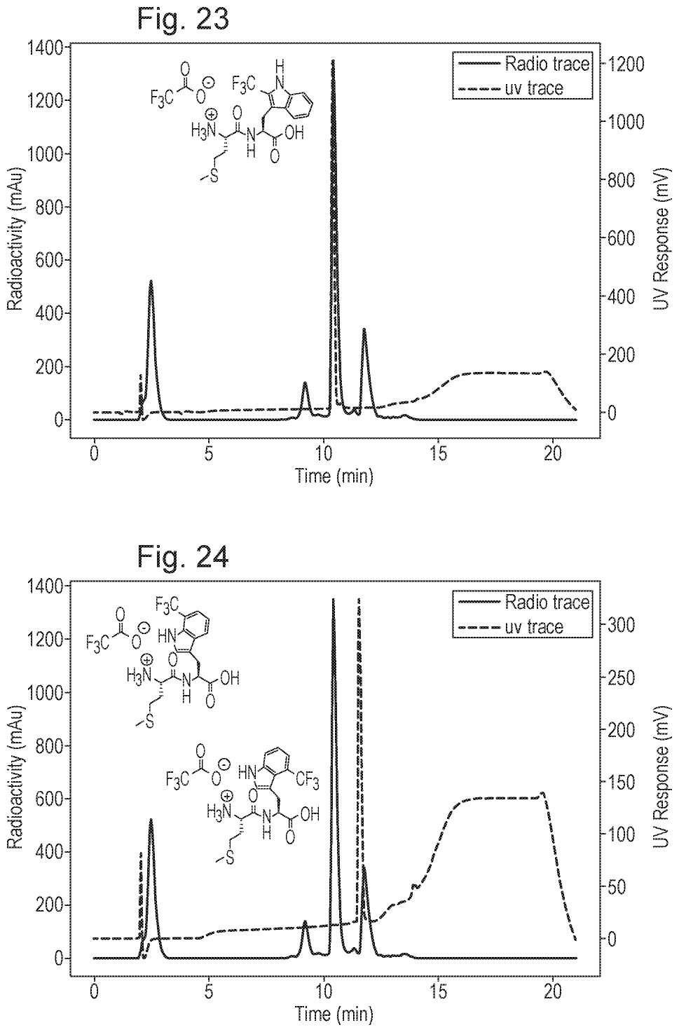

Prepared following the general procedure using H-Met-Trp-OH (0.03 mmol, 9.4 mg), Fe(NO₃)₃□9H₂O (0.06 mmol, 24.2 mg) and 70% TBHP in water (0.06 mmol, 8.3 μL), the amount of solvent is specified in Table 41. Radiochemical conversion was determined by radio-HPLC (Refer to Table 42 and Table 43). HPLC overlay spectra for the products are provided in FIGS. 23 and 24.

TABLE 41
Amount of solvent used for each reaction.

| | Amount of | |
|---|---|---|
| | [¹⁸F]NH₄SO₂CF₃ in | |
| | 25 mM | |
| | NH₄HCO₂ | Amount of |
| | dispensed/mL | DMSO added/mL |
| 1 | 0.10 | 0.15 |
| 2 | 0.10 | 0.15 |
| 3 | | |

TABLE 42
HPLC details and RCC for H-Met-Trp[2-CF₃]-OH

| Reaction | RCC^{a} (%) | HPLC column | Eluent |
|---|---|---|---|
| 1 | 35.36 | Synergi | A^{b} |
| 2 | 26.67 | Zorbax 300 Extended C-18 | A^{b} |
| 3 | 48.59 | Zorbax 300 Extended C-18 | A^{b} |
| Average | 36.9 | | |
| Sample Standard | 110 | | |
| Deviation | | | |

^{a}Determined from relative area of all radiopeaks in HPLC.
^{b}Refer to Table 11

TABLE 43
HPLC details and RCC for H-Met-Trp[
4-CF₃]-OH + H-Met-Trp[7-CF₃]-OH

| Reaction | RCC^{a} (%) | HPLC column | Eluent |
|---|---|---|---|
| 1 | 13.10 | Synergi | A^{b} |
| 2 | 9.58 | Zorbax 300 Extended C-18 | A^{b} |
| 3 | 16.04 | Zorbax 300 Extended C-18 | A^{b} |
| Average | 12.9 | | |
| Sample Standard | 3.2 | | |
| Deviation | | | |

^{a}Determined from relative area of all radiopeaks in HPLC.
^{b}Refer to Table 11

MetTyr[ortho-CF₂¹⁸F]

### (S)-2-((S)-2-amino-4-(methylthio)butanamido)-3-(4-hydroxy-3-([¹⁸F]trifluoromethyl)phenyl)propanoic acid

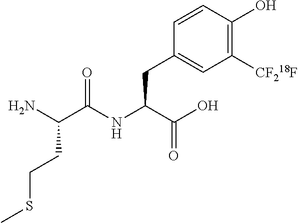

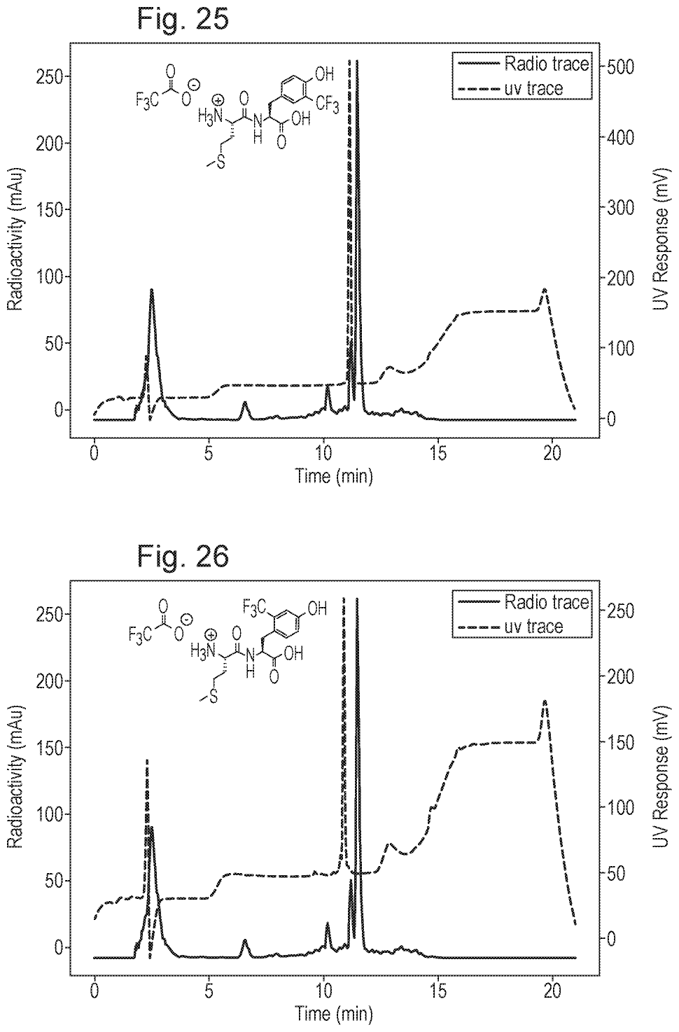

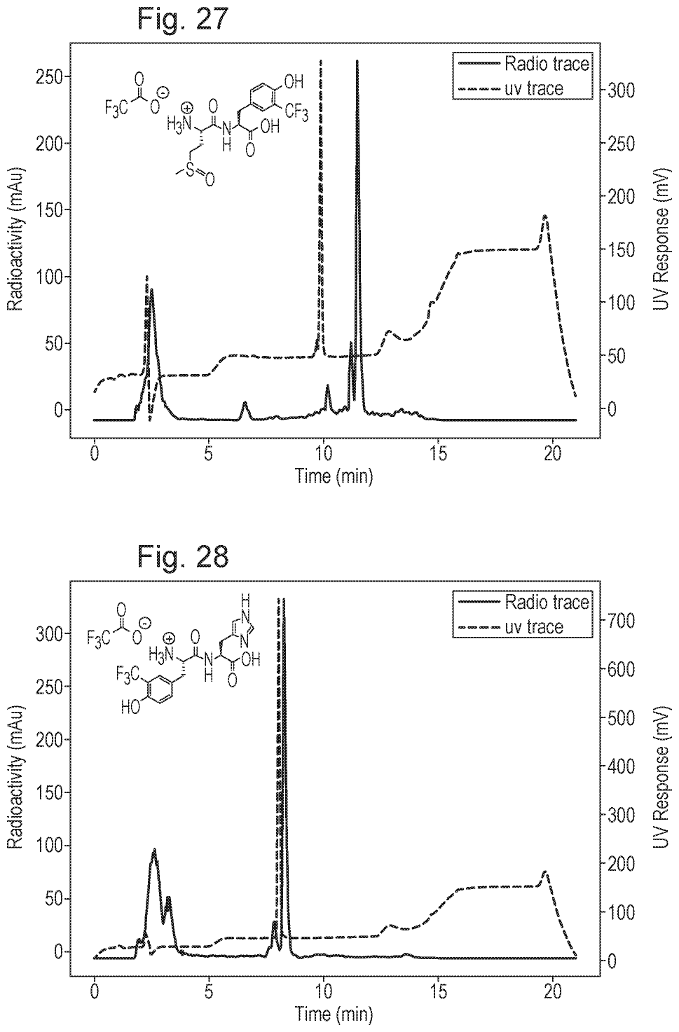

Prepared following the general procedure using H-Met-Tyr-OH (0.03 mmol, 9.4 mg), Fe(NO₃)₃□9H₂O (0.06 mmol, 24.2 mg) and 70% TBHP in water (0.06 mmol, 8.3 μL), the amount of solvent is specified in Table 44. Radiochemical conversion was determined by radio-HPLC (Refer to Table 45, Table 46 and Table 47). HPLC overlay spectra for the products are provided in FIGS. 25, 26 and 27.

TABLE 44
Amount of solvent used for each reaction.

| | Amount of | |
|---|---|---|
| | [¹⁸F]NH₄SO₂CF₃ in | |
| | 25 mM | Amount of |
| | NH₄HCO₂ | DMSO |
| | dispensed/mL | added/mL |
| 1 | 0.13 | 0.12 |
| 2 | 0.13 | 0.12 |
| 3 | 0.050 + 0.10 | 0.15 |

TABLE 45
HPLC details and RCC for H-Met-Tyr[ortho-CF3]-OH

| Reaction | RCC^{a} (%) | HPLC column | Eluent |
|---|---|---|---|
| 1 | 37.87 | Synergi | A^{b} |
| 2 | 34.61 | Zorbax 300 Extended C-18 | A^{b} |
| 3 | 32.34 | Synergi | A^{b} |
| Average | 34.9 | | |
| Sample Standard | 2.8 | | |
| Deviation | | | |

^{a}Determined from relative area of all radiopeaks in HPLC.
^{b}Refer to Table S11

TABLE 46
HPLC details and RCC for H-Met-Tyr[meta-CF₃]-OH

| Reaction | RCC^{a} (%) | HPLC column | Eluent |
|---|---|---|---|
| 1 | 7.47 | Synergi | A^{b} |
| 2 | 5.40 | Zorbax 300 Extended C-18 | A^{b} |
| 3 | 6.87 | Synergi | A^{b} |
| Average | 6.6 | | |
| Sample Standard | 1.1 | | |
| Deviation | | | |

^{a}Determined from relative area of all radiopeaks in HPLC.
^{b}Refer to Table S11

TABLE 47
HPLC details and RCC for H-Met(oxidized)-Tyr[CF₃]-OH

| Reaction | RCC^{a} (%) | HPLC column | Eluent |
|---|---|---|---|
| 1 | 8.05 | Synergi | A^{b} |
| 2 | 9.17 | Zorbax 300 Extended C-18 | A^{b} |
| 3 | 4.47 | Synergi | A^{b} |
| Average | 7.2 | | |
| Sample Standard | 2.5 | | |
| Deviation | | | |

^{a}Determined from relative area of all radiopeaks in HPLC.
^{b}Refer to Table S11

Tyr[ortho-CF₂¹⁸F]His

### 4-((S)-2-((S)-2-ammonio-3-(4-hydroxy-3-([¹⁸F]trifluoromethyl)phenyl)propanamido)-2-carboxyethyl)-1H-imidazol-3-Ium trifluoroacetate

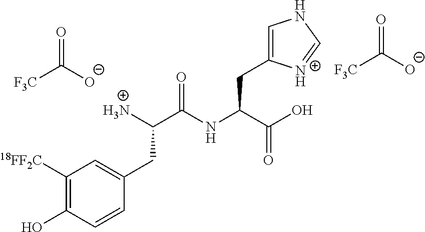

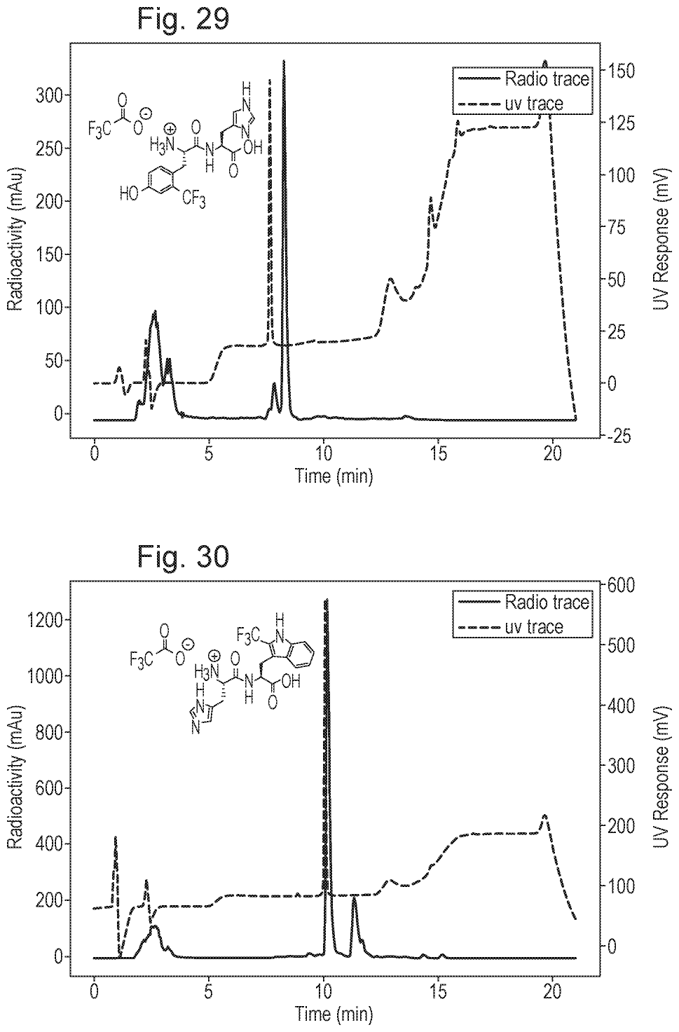

Prepared following the general procedure using H-Tyr-His-OH (0.03 mmol, 9.6 mg), Fe(NO₃)₃.9H₂O (0.06 mmol, 24.2 mg) and 70% TBHP in water (0.12 mmol, 16.6 μL), the amount of solvent is specified in Table 48. Radiochemical conversion was determined by radio-HPLC (Refer to Table 49 and Table 50). HPLC overlay spectra for the products are provided in FIGS. 28 and 29.

TABLE 48
Amount of solvent used for each reaction.

| | Amount of | | |
|---|---|---|---|
| | [¹⁸F]NH₄SO₂CF₃ | | |
| | in 25 mM | | |
| | NH₄HCO₂ | Amount of | |
| | dispensed/mL | added/mL | DMSO Total Volume/mL |
| 1 | 0.150 | 0.100 | 0.250 |
| 2 | 0.125 | 0.125 | 0.250 |
| 3 | 0.125 | 0.125 | 0.250 |

TABLE 49
HPLC details and RCC for H-Tyr[o-CF₃]-His-OH.

| Reaction | RCC^{a} (%) | HPLC column | Eluent |
|---|---|---|---|
| 1 | 34.49 | Synergi | A^{b} |
| 2 | 35.80 | Synergi | A^{b} |
| 3 | 32.78 | Synergi | A^{b} |
| Average | 34.4 | | |
| Sample Standard | 1.5 | | |
| Deviation | | | |

^{a}Determined from relative area of all radiopeaks in HPLC.
^{b}Refer to Table 11

TABLE 50
HPLC details and RCC for H-Tyr[m-CF₃]-His-OH.

| Reaction | RCC^{a} (%) | HPLC column | Eluent |
|---|---|---|---|
| 1 | 4.76 | Synergi | A^{b} |
| 2 | 5.42 | Synergi | A^{b} |
| 3 | 5.60 | Synergi | A^{b} |
| Average | 5.3 | | |
| Sample Standard | 0.4 | | |
| Deviation | | | |

^{a}Determined from relative area of all radiopeaks in HPLC.
^{b}Refer to Table 11

HisTrp[W-2-CF₂¹⁸F]

### 5-((S)-2-ammonio-3-(((S)-1-carboxy-2-(2-([¹⁸F]trifluoromethyl)-1H-indol-3-yl)ethyl)amino)-3-oxopropyl)-1H-imidazol-3-Ium trifluoroacetate

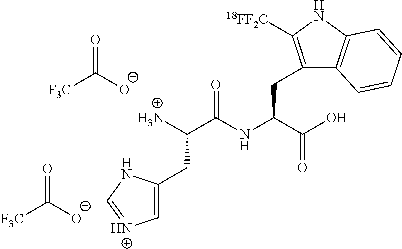

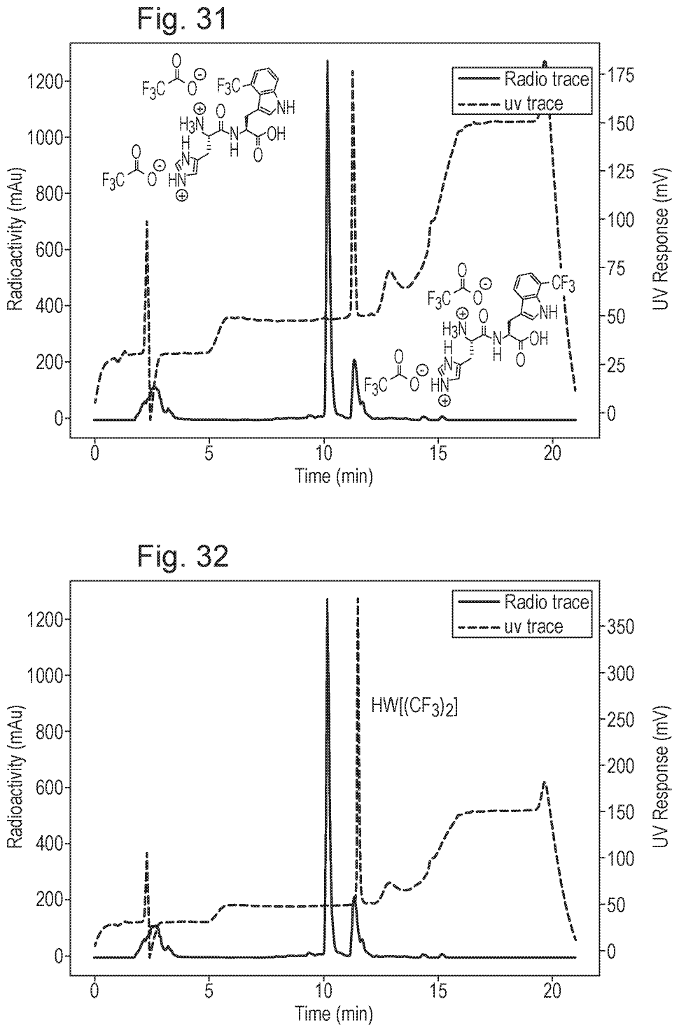

Prepared following the general procedure using H-His-Trp-OH (0.015 mmol, 5.1 mg), Fe(NO₃)₃.9H₂O (0.03 mmol, 12.1 mg) and 70% TBHP in water (0.06 mmol, 8.2 μL), the amount of solvent is specified in Table 51. Radiochemical conversion was determined by radio-HPLC (Refer to Table 52, Table 53 and Table 54). HPLC overlay spectra for the products are provided in FIGS. 30, 31 and 32.

TABLE 51
Amount of solvent used for each reaction.

| | Amount of [¹⁸F]NH₄SO₂CF₃ | | |
|---|---|---|---|
| | in 25 mM | | |
| | NH₄HCO₂ | Amount of | |
| | dispensed/mL | added/mL | DMSO Total Volume/mL |
| 1 | 0.06 | 0.1 | 0.16 |
| 2 | 0.06 | 0.1 | 0.16 |
| 3 | 0.03 | 0.1 | 0.13 |

TABLE 52
HPLC details and RCC for H-His-Trp[2-CF₃]-OH.

| Reaction | RCC^{a} (%) | HPLC column | Eluent |
|---|---|---|---|
| 1 | 47.65 | Synergi | A^{b} |
| 2 | 55.06 | Zorbax 300 Extended C-18 | A^{b} |
| 3 | 57.07 | Synergi | A^{b} |
| Average | 53.3 | | |
| Sample Standard | 5.0 | | |
| Deviation | | | |

^{a}Determined from relative area of all radiopeaks in HPLC.
^{b}Refer to Table S11

TABLE 53
HPLC details and RCC for H-His-Trp[4-CF₃]-OH
+ H-His-Trp[7-CF₃]-OH.

| Reaction | RCC^{a} (%) | HPLC column | Eluent |
|---|---|---|---|
| 1 | 12.42 | Synergi | A^{b} |
| 2 | 12.18 | Zorbax 300 Extended C-18 | A^{b} |
| 3 | 13.19 | Synergi | A^{b} |
| Average | 12.6 | | |
| Sample Standard | 0.5 | | |
| Deviation | | | |

^{a}Determined from relative area of all radiopeaks in HPLC.
^{b}Refer to Table S11

TABLE 54
HPLC details and RCC for H-His-Trp[diCF₃]-OH

| Reaction | RCC^{a} (%) | HPLC column | Eluent |
|---|---|---|---|
| 1 | 3.98 | Synergi | A^{b} |
| 2 | 3.83 | Zorbax 300 Extended C-18 | A^{b} |
| 3 | 4.05 | Synergi | A^{b} |
| Average | 4.0 | | |
| Sample Standard | 0.1 | | |
| Deviation | | | |

Glu-Trp[W-2-CF₂¹⁸F]

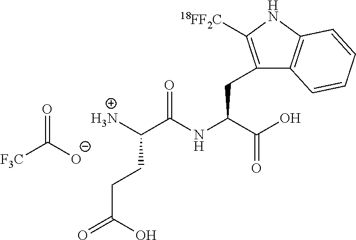

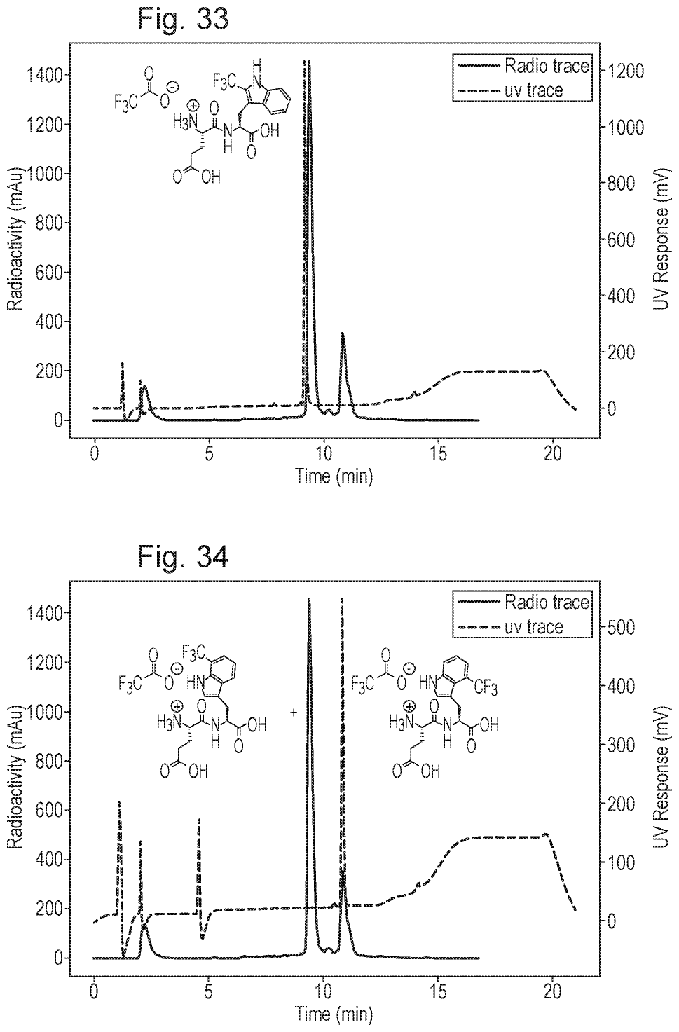

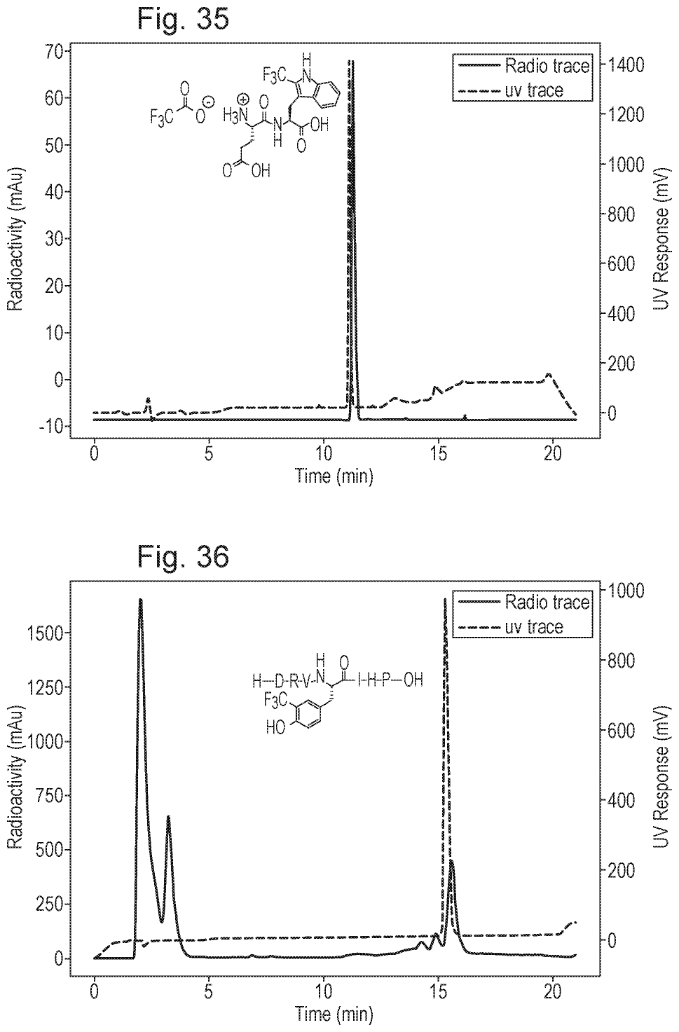

Prepared following the general procedure using H-Glu-Trp-OH (0.03 mmol, 10.0 mg), FeCl₃ (0.06 mmol, 9.7 mg) and 70% TBHP in water (0.06 mmol, 8.2 μL), the amount of solvent is specified in Table 55. Radiochemical conversion was determined by radio-HPLC (Refer to Table 56 and Table 57). HPLC overlay spectra for the products are provided in FIGS. 33, 34 and 35.

TABLE 55
Amount of solvent used for each reaction.

| | Amount of | | |
|---|---|---|---|
| | [¹⁸F]NH₄SO₂CF₃ | | |
| | in 25 mM | | |
| | NH₄HCO₂ | Amount of | |
| | dispensed/mL | added/mL | DMSO Total Volume/mL |
| 1 | 0.15 | 0.10 | 0.25 |
| 2 | 0.125 | 0.125 | 0.25 |
| 3 | 0.08 + 0.045 top-up^{a} | 0.125 | 0.25 |

^{a}Additonal 25 mM NH₄HCO₂ added beside those dispensed with the [¹⁸F]NH₄HCO₂.

TABLE 56
HPLC details and RCC for H-Glu-Trp[2-CF₃]-OH.

| Reaction | RCC^{a} (%) | HPLC column | Eluent |
|---|---|---|---|
| 1 | 70.08 | Zorbax 300 Extended C-18 | A^{b} |
| 2 | 66.34 | Zorbax 300 Extended C-18 | A^{b} |
| 3 | 69.86 | Zorbax 300 Extended C-18 | A^{b} |
| Average | 68.8 | | |
| Sample Standard | 2.1 | | |
| Deviation | | | |

^{a}Determined from relative area of all radiopeaks in HPLC.
^{b}Refer to Table S11

TABLE 57
HPLC details and RCC for H-Glu-Trp[4-CF₃]-OH
+ H-Glu-Trp[7-CF₃]-OH.

| Reaction | RCC^{a} (%) | HPLC column | Eluent |
|---|---|---|---|
| 1 | 19.31 | Zorbax 300 Extended C-18 | A^{b} |
| 2 | 21.95 | Zorbax 300 Extended C-18 | A^{b} |
| 3 | 23.63 | Zorbax 300 Extended C-18 | A^{b} |
| Average | 21.6 | | |
| Sample Standard | 2.2 | | |
| Deviation | | | |

^{a}Determined from relative area of all radiopeaks in HPLC.
^{b}Refer to Table S11

Isolation Results

TABLE 58
Isolation by HPLC results for Glu-Trp.

| | | Activity left | | | |
|---|---|---|---|---|---|
| | | in HPLC vial | | | |
| | Activity in | after | Injected | Isolated | |
| | HPLC vial | injection | Activity | activity | Radiochemical |
| Reaction | (MBq) | (MBq) | (MBq)^{a} | (MBq) | Yield (%)^{b} |
| 1 | 1.4 | 0.1 | 1.3 | 0.5 | 38.5 |
| 2 | 8.0 | 6.8 | 6.8 | 2.52 | 37.1 |
| 3 | 7.2 | 6.2 | 6.2 | 2.60 | 41.9 |
| Average | | | | | 39.2 |
| Sample | | | | | 2.5 |
| Standard | | | | | |
| Deviation | | | | | |

^{a}Due to the limitation of the HPLC loop only 100 μL can be injected each time. This limitation is operational and can be circumvented if required. ^{b}Radiochemical Yield = Isolated Activity/Injected Activity × 100 and no decay correction was applied. The Radiochemical purity of the isolated product is >99%. See Figure S41.

Angiotensin (1-7)[Y-ortho-CF₂¹⁸F]

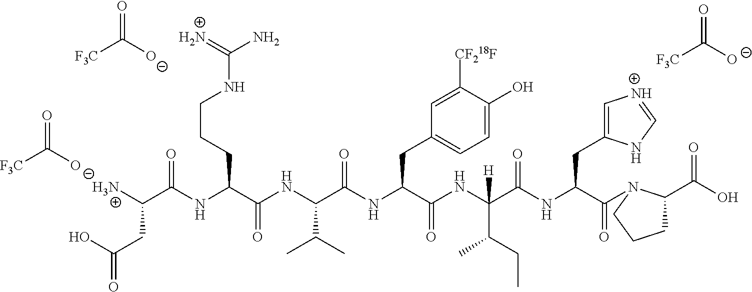

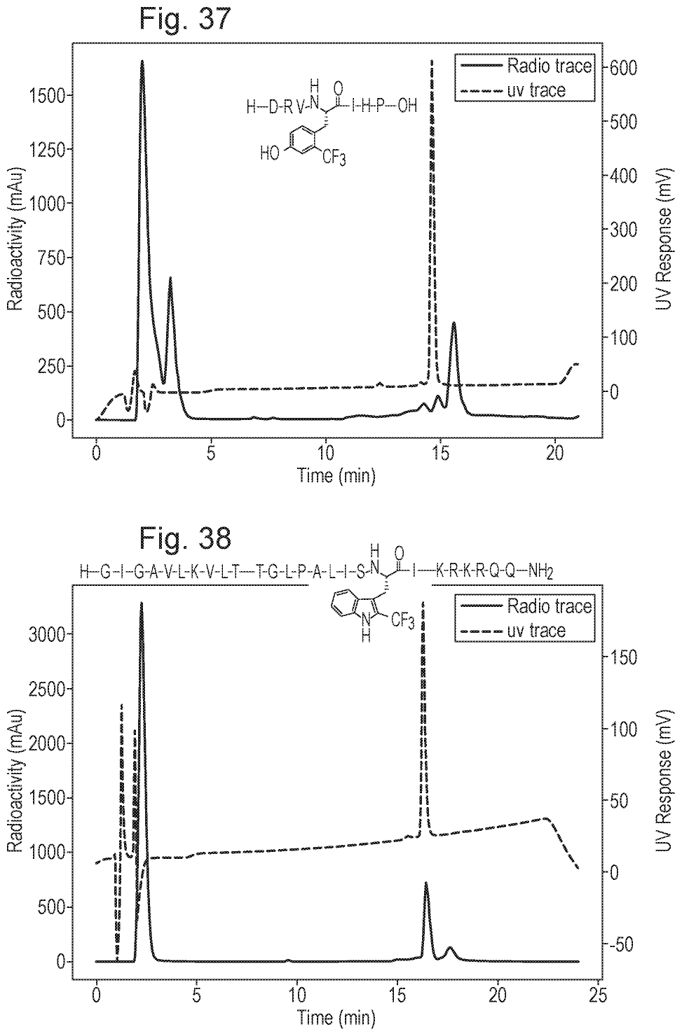

Prepared following the general procedure using angiotensin(1-7) (0.0092 mmol, 12 mg), Fe(NO₃)₃.9H₂O (0.06 mmol, 24.2 mg) and 70% TBHP in water (0.06 mmol, 8.2 μL), the amount of solvent is specified in Table 59. Radiochemical conversion was determined by radio-HPLC (Refer to Table 60 and Table 61). HPLC overlay spectra for the products are provided in FIGS. 36 and 37.

TABLE 59
Amount of solvent used for each reaction.

| | Amount of | | |
|---|---|---|---|
| | [¹⁸F]NH₄SO₂CF₃ | Amount of | |
| | in 25 mM NH₄HCO₂ | DMSO | |
| | dispensed/mL | added/mL | Total Volume/mL |
| 1 | 0.050 | 0.050 | 0.10 |
| 2 | 0.050 | 0.050 | 0.10 |
| 3 | 0.050 | 0.050 | 0.10 |

^{a} Additonal 25mM NH4HCO2 added beside those dispensed with the [¹⁸F]NH₄HCO2.

TABLE 60
HPLC details andRCC for Angiotensin (1-7) [Tyr-o-CF3].

| Reaction | RCC^{a} (%) | HPLC column | Eluent |
|---|---|---|---|
| 1 | 12.89 | Synergi | J^{b} |
| 2 | 19.94 | Synergi | J^{b} |
| 3 | 16.16 | Synergi | J^{b} |
| Average | 16.3 | | |
| Sample Standard | | | |
| Deviation | 3.5 | | |

^{a}Determined from relative area of all radiopeaks in HPLC.
^{b}Refer to Table 20.

TABLE 61
HPLC details and RCC for Angiotensin (1-7) [Tyr-m-CF3].

| Reaction | RCC^{a}(%) | HPLC column | Eluent |
|---|---|---|---|
| 1 | 2.40 | Synergi | J^{b} |
| 2 | 3.41 | Synergi | J^{b} |
| 3 | 2.81 | Synergi | J^{b} |
| Average | 2.9 | | |
| Sample Standard | | | |
| Deviation | 0.5 | | |

^{a}Determined from relative area of all radiopeaks in HPLC.
^{b}Refer to Table 20.

Melittin[W-2-CF₂¹⁸F]

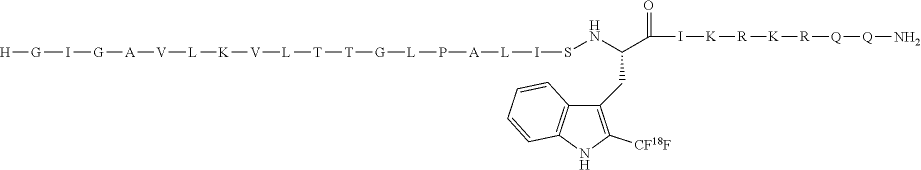

Prepared following the general procedure using Melittin (0.00074 mmol, 2.0 mg), FeCl₃ (0.02 mmol, 3.2 mg) and 70% TBHP in water (0.02 mmol, 2.8 μL), the amount of solvent is specified in Table 59. Radiochemical conversion was determined by radio-HPLC (Refer to Table 63). HPLC overlay spectra for the product is provided in FIG. 38.

TABLE 62
Amount of solvent used for each reaction.

| | Amount of | | |
|---|---|---|---|
| | [¹⁸F]NH₄SO₂CF₃ | Amount of | |
| | in 25 mM NH₄HCO₂ | DMSO | |
| | dispensed/mL | added/mL | Total Volume/mL |
| 1 | 0.015 + 0.010 top-up^{a} | 0.025 | 0.050 |
| 2 | 0.025 | 0.025 | 0.050 |
| 3 | 0.030 | 0.025 | 0.055 |

^{a}Additonal 25 mM NH₄HCO₂ added beside those dispensed with the [¹⁸F]NH₄HC0₂.

TABLE 63
HPLC details and RCC for Melittin [Trp-2-CF₃].

| Reaction | RCC? (%) | HPLC column | Eluent |
|---|---|---|---|
| 1 | 17.57 | Zorbax 300 Extended C-18 | F^{b} |
| 2 | 14.85 | Zorbax 300 Extended C-18 | F^{b} |
| 3 | 15.31 | Zorbax 300 Extended C-18 | F^{b} |
| Average | 15.91 | | |
| Sample Standard | 1.5 | | |
| Deviation | | | |

^{a}Determined from relative area of all radiopeaks in HPLC.
^{b}Refer to Table S16.

Somatostatin-14[W-2-CF₂¹⁸F]

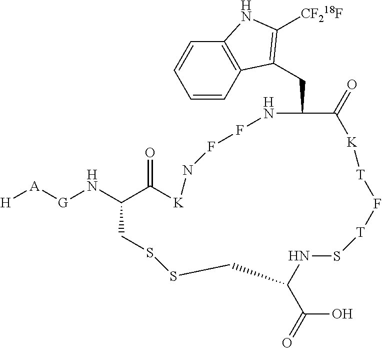

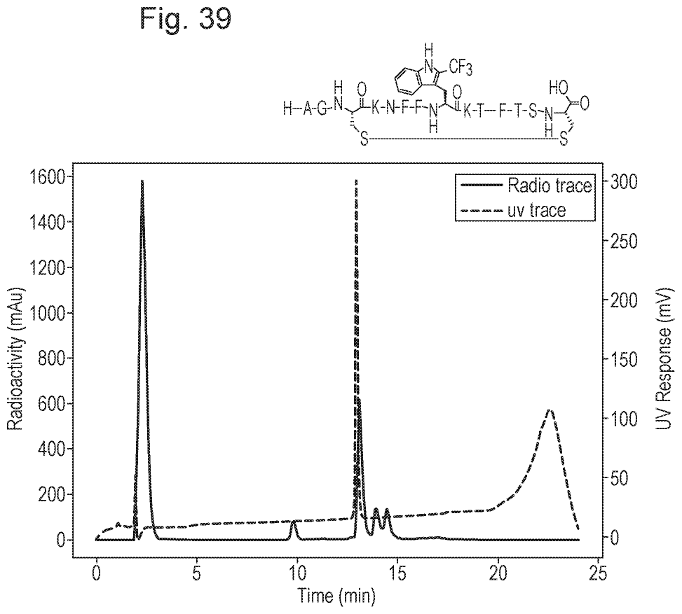

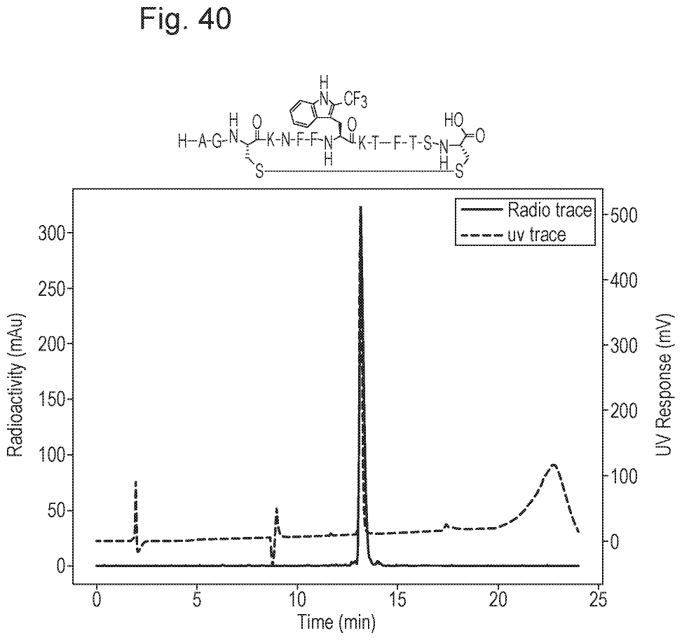

Prepared following the general procedure using Somatostatin-14 (0.0061 mmol, 10 mg), FeCl₃ (0.08 mmol, 13.0 mg) and 70% TBHP in water (0.08 mmol, 11 μL), the amount of solvent is specified in Table 64. Radiochemical conversion was determined by radio-HPLC (Refer to Table 65). HPLC overlay spectra for the products are provided in FIGS. 39 and 40.

TABLE 64
Amount of solvent used for each reaction.

| | Amount of | | |
|---|---|---|---|
| | [¹⁸F]NH₄SO₂CF₃ | Amount of | |
| | in 25 mM NH₄HCO₂ | DMSO | |
| | dispensed/mL | added/mL | Total Volume/mL |
| 1 | 0.075 | 0.075 | 0.15 |
| 2 | 0.050 | 0.100 | 0.15 |
| 3 | 0.050 | 0.100 | 0.15 |
| 4 | | | |

TABLE 65
HPLC details and RCC for Somatostatin-14 [W-2CF₃].

| Reaction | RCC^{a} (%) | HPLC column | Eluent |
|---|---|---|---|
| 1 | 19.06 | Zorbax 300 Extended C-18 | D^{b} |
| 2 | 8.23 | Zorbax 300 Extended C-18 | D^{b} |
| 3 | 8.30 | Zorbax 300 Extended C-18 | D^{b} |
| Average | | | |
| Sample Standard | | | |
| Deviation | | | |

^{a}Determined from relative area of all radiopeaks in HPLC.
^{b}Refer to Table 14.

Isolation Results

TABLE 66
Isolation by HPLC results for Somatostatin-14.

| | | Activity left | | | |
|---|---|---|---|---|---|
| | | in HPLC vial | | | |
| | Activity in | after | Injected | | |
| | HPLC vial | injection | activity | Isolated | Radiochemical |
| Reaction | (MBq) | (MBq) | (MBq)^{a} | Activity (MBq) | Yield (%)^{b} |
| 1 | 10.4 | 1.0 | 9.4 | 2.64 | 28.1 |
| 2 | 18.9 | 5.52 | 13.38 | 2.19 | 16.4 |
| Average | | | | | 22.2 |
| Sample | | | | | |
| Standard | | | | | 8.3 |
| Deviation | | | | | |

^{a}Due to the limitation of the HPLC loop only 100 μL can be injected each time. This limitation is operational and can be circumvented if required.
^{b}Radiochemical Yield = Isolated Activity/Injected Activity × 100 and no decay correction was applied. The Radiochemical purity of the isolated product is >99%. See FIG. 36.

Endomorphin 1[W-2-CF₂¹F]

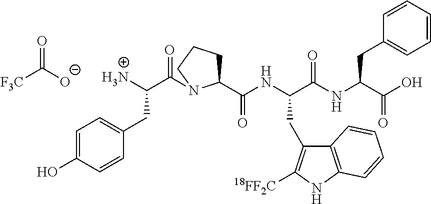

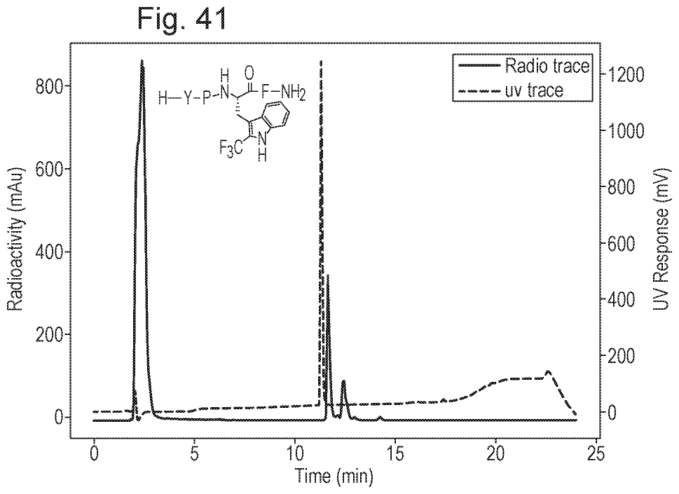

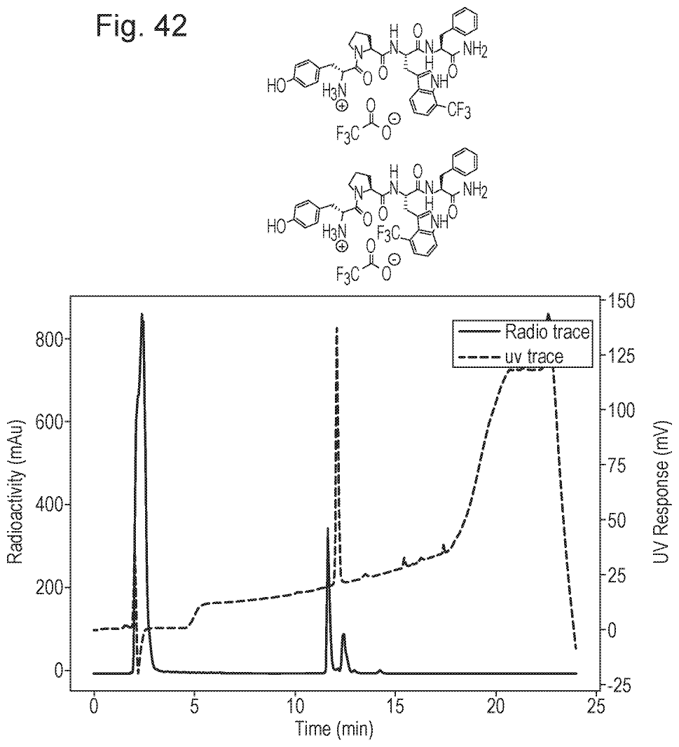

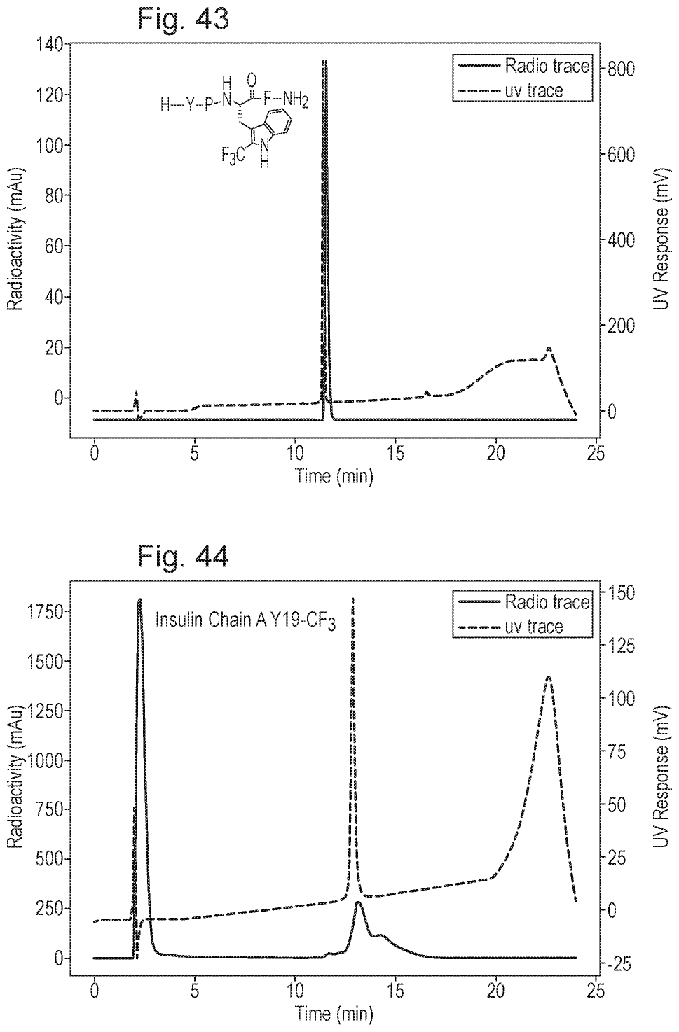

Prepared following the general procedure using Endomorphin 1 (0.004 mmol, 3 mg), FeCl₃ (0.02 mmol, 3.2 mg) and 70% TBHP in water (0.02 mmol, 2.8 μL), the amount of solvent is specified in Table 67. Radiochemical conversion was determined by radio-HPLC (Refer to Table 68 and Table 69). HPLC overlay spectra for the products are provided in FIGS. 41, 42 and 43.

TABLE 67
Amount of solvent used for each reaction.

| | Amount of | | |
|---|---|---|---|
| | [¹⁸F]NH₄SO₂CF₃ | Amount of | |
| | in 25 mM NH₄HCO₂ | DMSO | |
| | dispensed/mL | added/mL | Total Volume/mL |
| 1 | 0.015 + 0.035 topup^{a} | 0.050 | 0.1 |
| 2 | 0.050 | 0.050 | 0.1 |
| 3 | 0.060 | 0.040 | 0.1 |

^{a}Additonal 25 mM NH₄HCO₂ added beside those dispensed with the [¹⁸F]NH₄HCO₂.

TABLE 68
HPLC details and RCC for Endomorphin 1 [Trp-2-CF3].

| Reaction | RCC^{a}(%) | HPLC column | Eluent |
|---|---|---|---|
| 1 | 11.90 | Synergi | F^{b} |
| 2 | 15.52 | Synergi | F^{b} |
| 3 | 10.37 | Synergi | F^{b} |
| Average | 12.6 | | |
| Sample Standard | 2 6 | | |
| Deviation | | | |

^{a}Determined from relative area of all radiopeaks in HPLC.
^{b}Refer to Table 16.

TABLE 69
HPLC details and RCC for Endomorphin
1 [Trp-4-CF₃ and Trp-7-CF₃].

| Reaction | RCC^{a} (%) | HPLC column | Eluent |
|---|---|---|---|
| 1 | 5.20 | Synergi | F^{b} |
| 2 | 6.75 | Synergi | F^{b} |
| 3 | 4.39 | Synergi | F^{b} |
| Average | 5.4 | | |
| Sample Standard | | | |
| Deviation | 1.2 | | |

^{a}Determined from relative area of all radiopeaks in HPLC.
^{b}Refer to Table 16.

Isolation Results

TABLE 70
Isolation by HPLC results for Endomorphin 1.

| | | Activity left | | | |
|---|---|---|---|---|---|
| | | in HPLC vial | | | |
| | Activity in | after | Injected | | |
| | HPLC vial | injection | activity | Isolated | Radiochemical |
| Reaction | (MBq) | (MBq) | (MBq)^{a} | Activity (MBq) | Yield (%)^{b} |
| 1 | 6.12 | 0.3 | 5.82 | 0.7 | 12.0 |
| 2 | 14.6 | 1.6 | 13 | 2.92 | 22.5 |
| Average | | | | | 17.2 |
| Sample | | | | | |
| Standard | | | | | 7.4 |
| Deviation | | | | | |

^{a}Due to the limitation of the HPLC loop only 100 μL can be injected each time. This limitation is operational and can be circumvented if required.
^{b}Radiochemical Yield = Isolated Activity/Injected Activity × 100 and no decay correction was applied. The Radiochemical purity of the isolated product is >99%. See FIG. 39.

Insulin

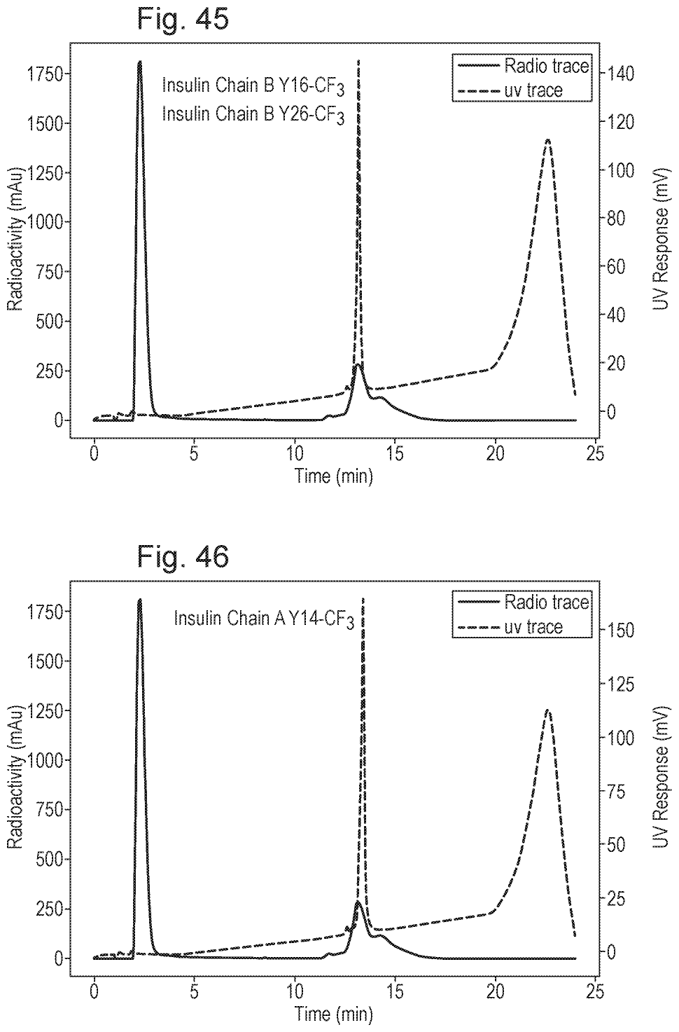

Prepared following the general procedure using recombinant human insulin (0.0052 mmol, 30 mg), Fe(NO₃)₃.9H₂O (0.03 mmol, 12.1 mg) and 70% TBHP in water (0.06 mmol, 8.2 μL), the amount of solvent is specified in Table 71. Radiochemical conversion was determined by radio-HPLC (Refer to Table 72). HPLC overlay spectra for the products are provided in FIGS. 44, 45 and 46.

TABLE 71
Amount of solvent used for each reaction.

| | Amount of | | |
|---|---|---|---|
| | [¹⁸F]NH₄SO₂CF₃ | | |
| | in 25 mM NH₄HCO₂ | Amount of | DMSO Total |
| | dispensed / mL | added/mL | Volume/mL |
| 1 | 0.035 | 0.10 | 0.135 |
| 2 | 0.050 | 0.10 | 0.15 |
| 3 | 0.030 | 0.12 | 0.15 |
| 4 | 0.025 + 0.050 top-up^{a} | 0.075 | 0.15 |
| 5 | | | |

^{a}Additonal 25 mM NH₄HCO₂ added beside those dispensed with the [¹⁸F]NH₄HCO₂.

TABLE 72
HPLC details and RCC for Insulin[Tyr-CF3].

| Reaction | RCC^{a} (%) | HPLC column | Eluent |
|---|---|---|---|
| 1 | 17.18 | Zorbax 300 Extended C-18 | A^{b} |
| 2 | 26.16 | Zorbax 300 Extended C-18 | G^{c} |
| 3 | 24.10 | Zorbax 300 Extended C-18 | D^{d} |
| 4 | 21.13 | Zorbax 300 Extended C-18 | D^{d} |
| 5 | 18.22 | Zorbax 300 SB CN | D^{d} |
| Average | 21.2 | | |
| Sample Standard | 2.9 | | |
| Deviation | | | |

^{a}Determined from relative area of all radiopeaks in HPLC.
^{b}Refer to Table 16.
^{c}Refer to Table 17.
^{d}Refer to Table 14.

### Synthesis of ¹⁸F-trifluormethyl functionalised cyclo(-Arg-Gly-Asp-D-Tyr-Lys)

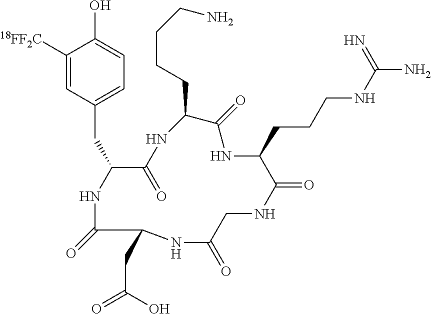

### ¹⁸F-trifluoromethyl functionalised cyclo(-Arg-Gly-Asp-D-Tyr-Lys)

To cyclo(-Arg-Gly-Asp-D-Tyr-Lys) (5 mg, 0.008 mmol) and iron(III) chloride hexahydrate (8.8 mg, 0.032 mmol) in a 3 mL V-vial was added [¹⁸F]NH₄SO₂CF₃ in aqueous NH₄HCO₂ solution (50-400 MBq) followed by tert-butyl hydroperoxide solution (8 μL, 0.06 mmol, 70% in H₂O) in DMSO to give a total reaction solvent of 75% DMSO in aqueous NH₄HCO₂ (200 μL) before stirring at 40° C. for 20 mins. An aliquot was removed for analysis by radio-HPLC to give a RCC.

Reverse phase HPLC details: 16% MecN in 84% NH₄HCO₂ 25 mM on a Phenomenex Synergi™ 4 μm Hydro RP 80 Å 150×4.6 mm column.

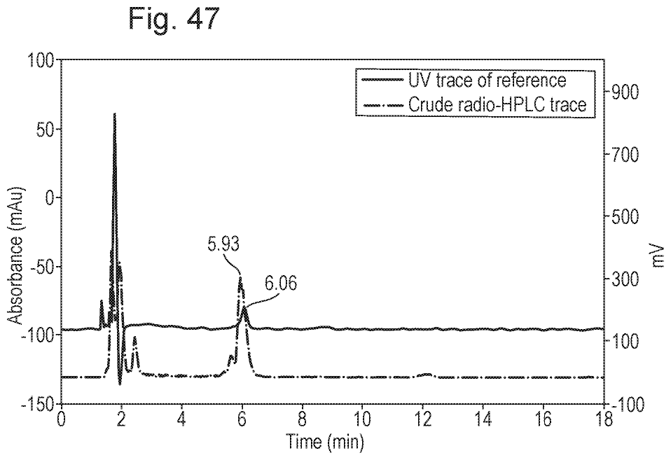

RCC (radiochemical conversion): 33%±15% (n=3, based on radio-HPLC analysis of the crude reaction, shown in FIG. 47).

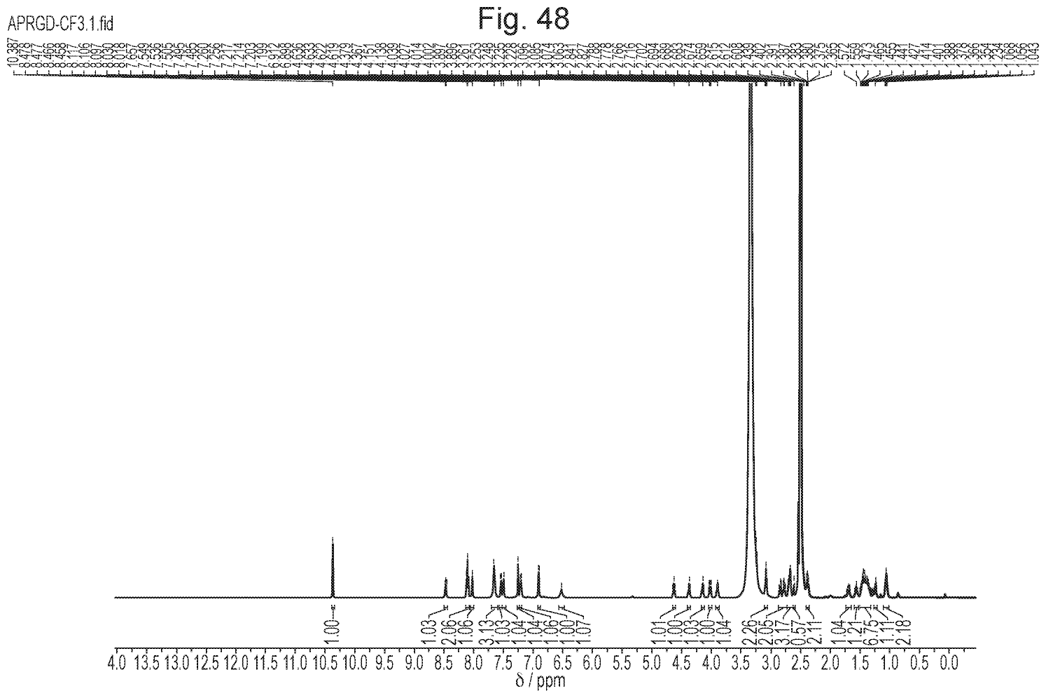

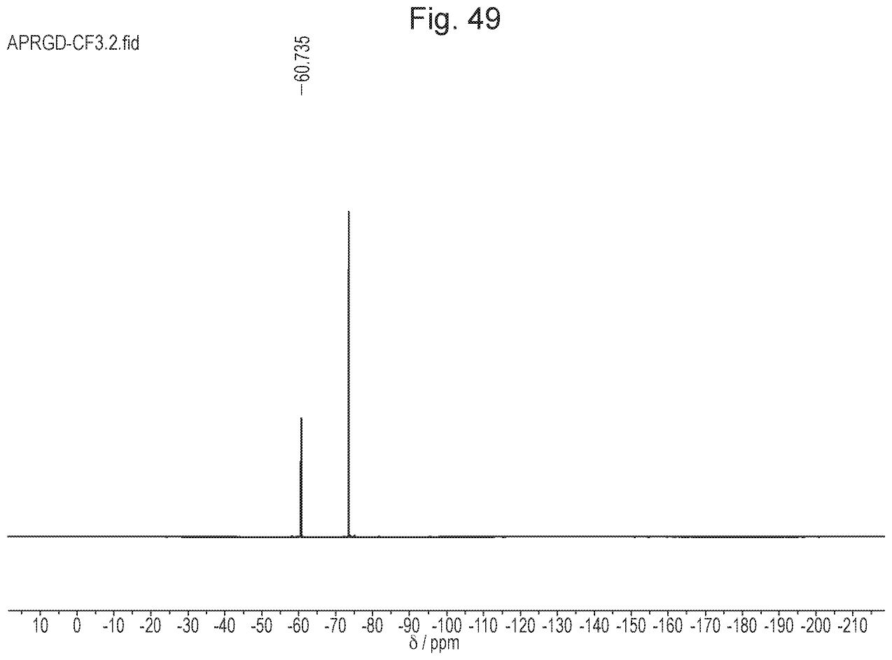

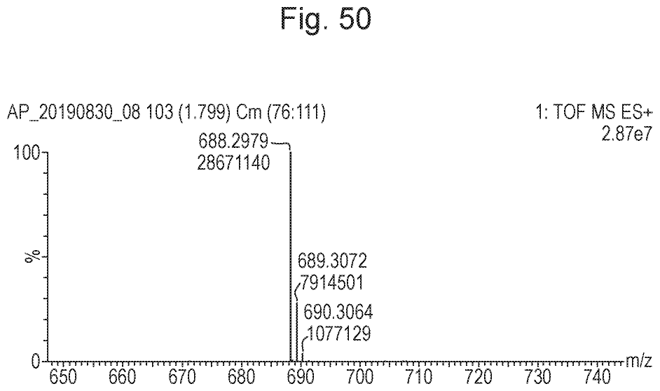

1H and 19F NMR, and TOF mass spectrometry results for the reference compound (¹⁹F-trifluoromethyl functionalised cyclo(-Arg-Gly-Asp-D-Tyr-Lys)) are shown in FIGS. 48, 49 and 50.

1. A process for producing a compound comprising the anion [CF₂¹⁸FSO₂]⁻, which process comprises treating a difluorocarbene source with (i) a source of ¹⁸F⁻ and (ii) a source of SO₂.

2. A process according to claim 1, wherein the difluorocarbene source provides difluorocarbene via an alpha elimination reaction.

3. A process according to claim 1, wherein the difluorocarbene source is a compound of Formula (I): 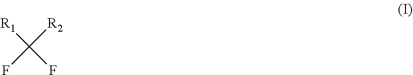
wherein R₁ is a first leaving group and R₂ is a second leaving group.

4. A process according to claim 3, wherein R₁ comprises a phosphonium or an ammonium cation.

5. (canceled)

6. (canceled)

7. A process according to claim 3, wherein R₂ is —C(═O)O⁻, or wherein R₂ is a group of formula —C(═O)OR₉, wherein R₉ is selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl,
or wherein R₉ is a group of formula —Si(R₁₀R₁₁R₁₂) wherein R₁₀, R₁₁ and R₁₂ are each independently selected from hydrogen, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, substituted or unsubstituted heteroaryl, C₁₋₂₀ alkoxy, aryloxy and halo,
or wherein R₂ comprises a carboxylate group.

8. A process according to claim 1 wherein the difluorocarbene source is (triphenylphosponio)difluoroacetate.

9. A process according to claim 1 wherein the source of SO₂ is:
(i) a compound of Formula (II): 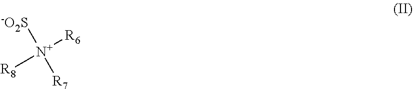
wherein R₆, R₇ and R₈ are each independently selected from H, substituted or unsubstituted C₁₋₂₀ alkyl, substituted or unsubstituted C₂₋₂₀ alkenyl, substituted or unsubstituted C₂₋₂₀ alkynyl, substituted or unsubstituted C₃₋₂₀ cycloalkyl, substituted or unsubstituted heterocyclyl, substituted or unsubstituted aryl, and substituted or unsubstituted heteroaryl;
provided that when at least two of R₆, R₇ and R₈ are substituted or unsubstituted C₁₋₂₀ alkyl groups, two of said substituted or unsubstituted C₁₋₂₀ alkyl groups may be bonded to a single heteroatom to form a ring, optionally wherein the heteroatom is O, S or N, wherein said N may be part of a group NR^{y} or N⁺R^{y}R^{z} wherein R^{y} is H, C₁₋₆ alkyl or aryl, and R^{z} is SO₂⁻, and
provided that when all three of R₆, R₇ and R₈ are substituted or unsubstituted C₁₋₂₀ alkyl groups, all three of said substituted or unsubstituted C₁₋₂₀ alkyl groups may be bonded to a single heteroatom, N, wherein said N may be part of a group N⁺R^{z} wherein R^{z} is H, C₁₋₆ alkyl, aryl or SO₂⁻, and preferably wherein R^{z} is SO₂⁻; or
(ii) a compound of formula (III): 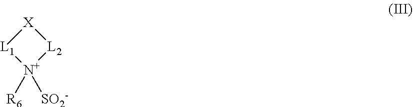
wherein X is selected from O, S, CH₂ and NH; L₁ and L₂ are substituted or unsubstituted C₁₋₆ alkylene, preferably substituted or unsubstituted C₂₋₆ alkylene; and R₆ is substituted or unsubstituted C₁₋₂₀ alkyl; or
(iii) N-methylmorpholine-SO₂; or
(iv) a compound of formula (IV): 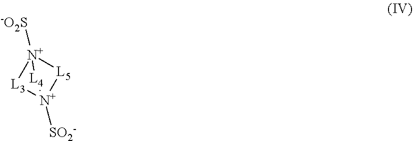
wherein L₃, L₄ and L₅ are selected from substituted or unsubstituted C₁₋₆ alkylene, preferably substituted or unsubstituted C₂₋₆ alkylene; or
(v) 1,4-diazabicyclo[2.2.2]octane bis(sulfur dioxide).

10-14. (canceled)

15. A process according to claim 1 wherein the compound comprising the anion is [CF218FSO₂]−nAn+, wherein n is an integer of from 1 to 4.

16. (canceled)

17. A process according to claim 15 wherein the step of treating the difluorocarbene source with the source of ¹⁸F⁻ and the source of SO₂ is performed:
(a) in the presence of Aⁿ⁺; or
(b) in the presence of a first cation B^{m+} to produce a compound of formula [CF₂¹⁸FSO₂]⁻ₘB^{m+}, wherein m is an integer of from 1 to 4, and the process further comprises replacing the first cation B^{m+} with a different cation Aⁿ⁺, to produce said compound of formula [CF₂¹⁸FSO₂]⁻ₙAⁿ⁺.

18-23. (canceled)

24. A process according to claim 1 which comprises treating the difluorocarbene source with at least 2 GBq of the ¹⁸F−.

25-26. (canceled)

27. A compound comprising the anion [CF₂¹⁸FSO₂]⁻.

28. A compound according to claim 27 wherein the compound comprising the anion is [CF₂¹⁸FSO₂]⁻ₙAⁿ⁺, wherein n is an integer of from 1 to 4.

29. A compound according to claim 28 wherein n is 1, wherein A is an alkali metal cation or an ammonium cation.

30. A compound according to claim 27 wherein the compound is obtained by a process which comprises treating a difluorocarbene source with (i) a source of ¹⁸F⁻ and (ii) a source of SO₂.

31. A process for producing a compound comprising an ¹⁸F− trifluoromethyl functionalised aromatic group, which process comprises contacting a compound comprising an aromatic group with a compound comprising the anion [CF218FSO₂]− in the presence of an activator for trifluoromethyl radical formation.

32-44. (canceled)

45. A process according to claim 31 which further comprises obtaining the compound comprising the anion [CF218FSO2]− by a process which comprises treating a difluorocarbene source with (i) a source of ¹⁸F⁻ and (ii) a source of SO₂.

46. A compound comprising an ¹⁸F-trifluoromethyl functionalised aromatic group.

47-50. (canceled)

51. A compound according to claim 46 wherein said compound is Thymogen in which tryptophan is functionalised with a ¹⁸F-trifluoromethyl group, Endomorphin I in which tryptophan is functionalised with a ¹⁸F-trifluoromethyl group, Melittin in which tryptophan is functionalised with a ¹⁸F-trifluoromethyl group, Angiotensin I/II in which tyrosine is functionalised with a ¹⁸F-trifluoromethyl group, insulin in which tyrosine is functionalised with a ¹⁸F-trifluoromethyl group, somatostatin-14 in which tryptophan is functionalised with a ¹⁸F-trifluoromethyl group, or cyclo(-Arg-Gly-Asp-D-Tyr-Lys) in which tyrosine is functionalised with a ¹⁸F-trifluoromethyl group.

52. A compound according to claim 46 wherein said compound is obtained by a process which comprises contacting a compound comprising an aromatic group with a compound comprising the anion [CF₂¹⁸FSO₂]⁻ in the presence of an activator for trifluoromethyl radical formation.

53. (canceled)

54. A method of imaging a subject, comprising administering to the subject a compound comprising an ¹⁸F-trifluoromethyl functionalised aromatic group or a pharmaceutically acceptable salt thereof, and imaging the subject by positron emission tomography (PET).
